(19) United States
(12) Patent Application Publication
(10) Pub. No.: US 2019/0062697 A1
(43) Pub. Date: Feb. 28, 2019

(54) Cell Differentiation Marker and its Uses

(71) Applicant: Centre National De La Recherche Scientifique (CNRS), Paris cedex 16 (FR)

(72) Inventors: Domenico Maiorano, Saint Martin De Londres (FR); Siem Van Der Laan, Granges (FR)

(21) Appl. No.: 15/998,535

(22) Filed: Aug. 16, 2018

Related U.S. Application Data
(63) Continuation of application No. 15/029,742, filed on Apr. 15, 2016, now abandoned.

(30) Foreign Application Priority Data
Oct. 21, 2013 (EP) 13306448.5

(51) Int. Cl. C12N 5/0735; A61K 31/44; C12N 15/113; A61K 38/48; C12N 9/48

(52) U.S. Cl. CPC C12N 5/0606; C12N 2501/90; C12N 2320/30; C12N 2501/734; A61K 31/44; C12N 15/1137; C12N 2310/14; C12Y 304/19012; A61K 38/4813; C12N 2501/998; C12N 9/485; C12N 2502/99

(57) ABSTRACT

The use of Dub3 protein, a nucleic acid molecule coding for the protein, or an inhibitor of the activity and/or of the expression of the protein for modulating cell differentiation.

### FIELD OF THE INVENTION

The present invention relates to a cell differentiation marker, in particular a totipotent/pluripotent stem cell marker, and its uses.

### BACKGROUND OF THE INVENTION

Eukaryotic cells have developed checkpoints to block cell cycle progression upon DNA damage or replication stress. Two distinct pathways pertain to the G1/S checkpoint by directly reducing CDK2 activity: a) rapid destruction of the Cdc25A phosphatase resulting in increased CDK2 phosphorylation, and b) a slower, p53-mediated, transcriptional response that activates expression of, amongst others, the potent CDK2 inhibitor p21. Importantly, rapid p21 degradation observed after exposure to low UV doses may be important for optimal DNA repair, while inhibition of CDK2 activity following Cdc25A degradation is sufficient for cell cycle arrest. Cdc25A protein levels are tightly regulated by two E3 ubiquitin ligases, the Anaphase Promoting Complex/Cyclosome (APC/CCdh1) as cells exit mitosis, and the Skp1-Cullin1-Fbox (SCF^{β-TrCP}) during both S and G2 phase and following DNA damage.

Compared to somatic cells, mouse embryonic stem (ES) cells appear to have a relaxed G1/S checkpoint. The molecular mechanism underlying this feature remains unclear. Moreover, mouse ES cell cycle has remarkably short G1 and G2 phases, with little S phase length variation. This is underpinned by high CDK2/Cyclin E activity and reduced APC/C activity leading to limited oscillation in substrate levels. Interestingly, knockdown of CDK2 protein was shown to increase G1 length although DNA damage-dependent degradation of Cdc25A was reported not to affect CDK2 activity, nor to induce a G1 arrest.

Maintenance of pluripotency depends upon expression of pluripotency genes under the combinatorial control of a regulatory network of transcription factors such as Nanog, Sox2 and Oct4. Differentiation of ES cell induces cell cycle remodelling, including appearance of longer G1 and G2 phases, but how this regulation is achieved is unknown. Moreover, how the pluripotency regulatory network impacts onto cell cycle control remains obscure. Aside from its well-known role in somatic cell cycle, very little is known about Cdc25A function in ES cells. In human ES cells, Cdc25A expression was shown to be regulated by Nanog. A recent report shows that Nanog knockdown in mouse ES cells results in G1/S transition delay by an unknown mechanism. Equally, the role of p53 in ES cells G1/S DNA damage checkpoint still remains controversial. Despite its high abundance, p53 has been proposed to be inactive in ES cells due to a predominant cytoplasmic distribution.

However, pluripotency markers that are highly specific for pluripotent cells remain to be identified, and the purification of a homogenous population of stem cells, or totipotent/pluripotent cells is still difficult to achieve.

Therefore there is a need to provide new pluripotecy/totipotency markers to allow isolation of the most undifferentiated cells among a cell population of differentiated cells.

One aim of the invention is to provide a new differentiation marker expressed in undifferentiated cells.

Another aim of the invention is to regulate cell differentiation of pluripotent/totipotent cells.

Still another aim of the invention is to provide cells expressing such differentiation marker, and process for obtaining them.

### SUMMARY OF THE INVENTION

The invention relates to the use of:
the Dub3 protein, said protein comprising the amino acid sequence as set forth in SEQ ID NO: 1, or any variant thereof having at least 43% identity with said amino acid sequence SEQ ID NO: 1, and having ubiquitin hydrolase activity or
a nucleic acid molecule coding for said protein or said variant thereof, or
an inhibitor of the activity and/or of the expression of said protein or said variant thereof,

for modulating cell differentiation, in particular in vitro cell differentiation.

The invention is based on the unexpected observation made by the inventors that the presence or the amount of Dub3 protein is able to modulate cell differentiation state.

In other words, the inventors have demonstrated that Dub3 protein, or its variant, or nucleic acid coding them, or inhibitor of said protein and variant can modulate the differentiation status of determined cells.

Reversible modification of target proteins with ubiquitin regulates an assortment of signaling pathways either through proteasomal degradation or by altering the activity and/or localization of constituent proteins. Ubiquitin conjugation is mediated via an E1-E2-E3 cascade, whereas ubiquitin removal is catalyzed by deubiquitinating enzymes (Dubs). The deconjugation reactions are performed by specific cysteine proteases which generate monomeric ubiquitin from a variety of C-terminal adducts. Deubiquitinating enzymes (DUBs) are the largest family of enzymes in the ubiquitin system with diverse functions, making them key regulators of ubiquitin-mediated pathways and they often function by direct or indirect association with the proteasome. The activity of DUBs has been implicated in several important pathways including cell growth, oncogenesis, neuronal disease and transcriptional regulation. DUBs catalyze the removal of ubiquitin from native conjugates, ubiquitin C-terminal extension peptides and linear poly-ubiquitin fusion or precursor proteins. DUBs are classed into two distinct families: ubiquitin C-terminal hydrolases (UCHs) and the ubiquitin-specific proteases (USPs/UBPs). UCHs are relatively small enzymes (20-30 kDa) that catalyze the removal of peptides and small molecules from the C-terminus of ubiquitin. Most UCHs cannot generate monomeric ubiquitin from protein conjugates or disassemble poly-ubiquitin chains.

Human Dub3, also called ubiquitin specific peptidase 17-like family member 2, comprises or consists of the amino acid sequence as set forth SEQ ID NO: 1.

In the invention, expression “for modulating cell differentiation” means both “for inducing differentiation” and “maintaining cell differentiation”.

According to the invention, “modulating cell differentiation” should also be interpreted as “modulating cell differentiation status”. Modulating cell differentiation status means that a determined cell, which is at a determined state of differentiation, can be
either maintained in said state of differentiation, by inhibiting cell differentiation,
or engaged towards differentiation, by activating cell differentiation.

In other words, by modulating cell differentiation state, the compounds according to the invention can
either stimulate cell differentiation, i.e. a less specialized cell becomes a more specialized cell type,
or inhibit cell differentiation, i.e. cells are maintained at a determined differentiation state despite extra or intracellular signals inducing cell differentiation,
or reverse cell differentiation, i.e. a more specialized cell type becomes a less specialized cell type, by dedifferentiation.

According to the invention, any variant of Dub3 protein having at least 43% identity with the amino acid sequence SEQ ID NO: 1, and having ubiquitin hydrolase activity can also modulate cell differentiation state.

By at least 43% identity, it is meant that the variants encompassed by the invention can have 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identity with the amino acid sequence SEQ ID NO: 1.

Advantageous Dub3 variants according to the inventions comprise or consist of the amino acid sequences as set forth in SEQ ID NO: 2 to SEQ ID NO: 19, i.e. SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 and SEQ ID NO: 19.

The above variants also harbor ubiquitin hydrolase activity, in particular deubiquitinase activity. This activity can be measured as described in Burrows et al, 2004, JBC, 279(14), 13993-14000. Briefly, the deubiquitination assay is based on the cleavage of ubiquitin-β-galactosidase (substrate) fusion proteins. Dub3 open reading frame (amino acids 1 to 530 of SEQ ID NO: 1), or variant thereof, and an equivalent open reading frame containing a catalytically inactive mutant form, Dub3C/S (C89S), or variant thereof, are generated by PCR and inserted in-frame into the pGEX vector in-frame with the glutathione S-transferase epitope. Ub-Met-β-galactosidase is expressed from a pACYC184-based plasmid. Plasmids are co-transformed into MC1061 Escherichia coli stain. Plasmid-bearing E. coli MC1061 cells are lysed and proteins analyzed by immunoblotting with a rabbit anti-β-galactosidase antiserum for detecting the substrate. Proteins are separated by SDS PAGE with a high density bisacrylamide-acrylamide gel to distinguish Ub-Met-β-galactosidase (un cleaved) and -β-galactosidase (cleaved) substrates. Protocol is also available in Papa et al. 1993, vol. 366, 313-319.

Therefore, the skilled person, by measuring the ability of the variants to deubiquitinate the Ub-Met-β-galactosidase substrate, can easily determine that a variant of Dub3 harbors deubiquitinase activity, i.e. ubiquitin hydrolase activity.

According to the invention, a nucleic acid molecule coding for said protein or said variant thereof is a nucleic acid that contain the nucleic information allowing the translation into said protein or said variant thereof, taking account of the genetic code degeneracy.

Advantageously, the nucleic acid coding the protein consisting of SEQ ID NO: 1 comprises the nucleic acid sequence as set forth in SEQ ID NO: 20.

Advantageously, the nucleic acid coding the protein consisting of SEQ ID NO: 2 comprises the nucleic acid sequence as set forth in SEQ ID NO: 21.

Advantageously, the nucleic acid coding the protein consisting of SEQ ID NO: 3 comprises the nucleic acid sequence as set forth in SEQ ID NO: 22.

Advantageously, the nucleic acid coding the protein consisting of SEQ ID NO: 4 comprises the nucleic acid sequence as set forth in SEQ ID NO: 23.

Advantageously, the nucleic acid coding the protein consisting of SEQ ID NO: 5 comprises the nucleic acid sequence as set forth in SEQ ID NO: 24

Advantageously, the nucleic acid coding the protein consisting of SEQ ID NO: 6 comprises the nucleic acid sequence as set forth in SEQ ID NO: 25

Advantageously, the nucleic acid coding the protein consisting of SEQ ID NO: 7 comprises the nucleic acid sequence as set forth in SEQ ID NO: 26

Advantageously, the nucleic acid coding the protein consisting of SEQ ID NO: 8 comprises the nucleic acid sequence as set forth in SEQ ID NO: 27

Advantageously, the nucleic acid coding the protein consisting of SEQ ID NO: 9 comprises the nucleic acid sequence as set forth in SEQ ID NO: 28

Advantageously, the nucleic acid coding the protein consisting of SEQ ID NO: 10 comprises the nucleic acid sequence as set forth in SEQ ID NO: 29

Advantageously, the nucleic acid coding the protein consisting of SEQ ID NO: 11 comprises the nucleic acid sequence as set forth in SEQ ID NO: 30

Advantageously, the nucleic acid coding the protein consisting of SEQ ID NO: 12 comprises the nucleic acid sequence as set forth in SEQ ID NO: 31

Advantageously, the nucleic acid coding the protein consisting of SEQ ID NO: 13 comprises the nucleic acid sequence as set forth in SEQ ID NO: 32

Advantageously, the nucleic acid coding the protein consisting of SEQ ID NO: 14 comprises the nucleic acid sequence as set forth in SEQ ID NO: 33

Advantageously, the nucleic acid coding the protein consisting of SEQ ID NO: 15 comprises the nucleic acid sequence as set forth in SEQ ID NO: 34

Advantageously, the nucleic acid coding the protein consisting of SEQ ID NO: 16 comprises the nucleic acid sequence as set forth in SEQ ID NO: 35

Advantageously, the nucleic acid coding the protein consisting of SEQ ID NO: 17 comprises the nucleic acid sequence as set forth in SEQ ID NO: 36

Advantageously, the nucleic acid coding the protein consisting of SEQ ID NO: 18 comprises the nucleic acid sequence as set forth in SEQ ID NO: 37

Advantageously, the nucleic acid coding the protein consisting of SEQ ID NO: 19 comprises the nucleic acid sequence as set forth in SEQ ID NO: 38

According to the invention, an inhibitor of the activity, i.e. of the ubiquitin hydrolase activity of Dub3 or a variant thereof can be chosen among the well-known compounds inhibiting such activity. An advantageous inhibitor is the PR-619 inhibitor, having the following formula I:

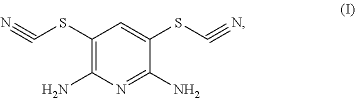 [SMILES: N#CSc1cc(SC#N)c(N)nc1N]

which is available from Sigma Aldrich (ref: SML0430). PR-619 is a cell permeable broad spectrum deubiquitylating enzymes (DUBs) inhibitor. PR-619 induces the accumulation of polyubiquitylated proteins in cells without directly affecting proteasome activity.

Inhibitory effect of such inhibitor can be measured as mentioned above. Specific antibodies, which for instance recognize catalytic domain of Dub3, or variant thereof, can also be used for the purpose of the invention. Antibodies, monoclonal or polyclonal, obtained by immunization of animal with the peptide consisting of SEQ ID NO: 39 are advantageous.

According to the invention, an inhibitor of expression of Dub3 or a variant thereof can be chosen among miRNA, siRNA, shRNA, or antisense nucleic acid molecules specific to the Dub3 or variant thereof sequence.

Another aspect of the invention concerns a method for modulating cell differentiation, in particular in vitro, comprising a step of introduction in a cell for which a modification of the differentiation state is required of an effective amount of
the Dub3 protein, said protein comprising the amino acid sequence as set forth in SEQ ID NO: 1, or any variant thereof having at least 43% identity with said amino acid sequence SEQ ID NO: 1, and having ubiquitin hydrolase activity or
a nucleic acid molecule coding for said protein or said variant thereof, or
an inhibitor of the activity and/or of the expression of said protein or said variant thereof.

Advantageously, the invention relates to the use as defined above, wherein said cell is totipotent or pluripotent cell. Thus, the invention advantageously relates to the use as defined above for modulating totipotent and multipotent cell differentiation, in particular in vitro totipotent and multipotent cell differentiation.

Totipotent stem cells can differentiate into embryonic and extra-embryonic cell types. Pluripotent stem cells originate from totipotent cells and can give rise to progeny that are derivatives of the three embryonic germ layers, mesoderm, ectoderm and endoderm.

Another aspect of the invention concerns a method for modulating totipotent or pluripotent cell differentiation, in particular in vitro, comprising a step of introduction in a cell for which a modification of the differentiation state is required of an effective amount of
the Dub3 protein, said protein comprising the amino acid sequence as set forth in SEQ ID NO: 1, or any variant thereof having at least 43% identity with said amino acid sequence SEQ ID NO: 1, and having ubiquitin hydrolase activity or
a nucleic acid molecule coding for said protein or said variant thereof, or
an inhibitor of the activity and/or of the expression of said protein or said variant thereof.

The invention also relates to the use of
Dub3 protein, said protein comprising the amino acid sequence as set forth in SEQ ID NO: 1, or any variant thereof having at least 43% identity with said amino acid sequence SEQ ID NO: 1, or
a nucleic acid molecule coding for said protein or said variant thereof,

for inducing dedifferentiation of differentiated cells, the cells obtained from the dedifferentiation of differentiated cells being iPS cells.

The inventors have observed that Dub3 protein is expressed in stem cells, and progressively disappears during differentiation process. They postulate that enforced expression of Dub3 would, in association with other genes, induce a dedifferentiation of somatic cells.

Induced pluripotent stem cells, commonly abbreviated as iPS cells or iPSCs are a type of pluripotent stem cell artificially derived from a non-pluripotent cell—typically an adult somatic cell—by inducing a “forced” expression of specific genes. Induced pluripotent stem cells are similar to natural pluripotent stem cells, such as embryonic stem (ES) cells, in many aspects, such as the expression of certain stem cell genes and proteins, chromatin methylation patterns, doubling time, embryoid body formation, teratoma formation, viable chimera formation, and potency and differentiability.

Advantageously, the invention relates to the use as defined above, for inducing dedifferentiation of differentiated cells, wherein said cells Dub3 protein, or a variant thereof, or said nucleic acid molecule coding for said protein, or said variant thereof, is associated with at least an Oct family member protein and a Sox family member protein.

According to this embodiment, iPS cells are obtained by allowing the expression, in a somatic differentiated cell, of at least Oct4 protein and a Sox2 protein, along with at Dub3 protein.

Advantageously, iPS cells can be obtained, from differentiated cells expressing Oct4/Sox2 and Dub3 genes, in particular expressing Oct4/Sox2/cMyc and Dub3 genes.

In one advantageous embodiment, the invention relates to the use as defined above, wherein said Dub3 protein or a variant thereof, or said nucleic acid molecule coding for said protein, or said variant thereof, is expressed in said iPS cells at a level corresponding to at least 2 fold lower than the expression of said Dub3 protein in totipotent or pluripotent cells.

It is possible to measure the expression of Dub3 by quantitative determination of Dub3 mRNA abundance by RT-PCR, one example of which is provided in FIG. 7A and/or by detection of the Dub3 protein by western blot using a specific antibody, such as one described in FIG. 11E.

The advantage of this level of expression being that said iPS cells will be now able to efficiently respond to DNA damage and/or replication stress generated by ectopic expression of factors such as c-myc or Oct family proteins, required for generating said iPS cells and thereby preserving genomic stability by reduction of CDK2 activity and resulting delay in the G1 phase of the cell cycle.

Such effect is exemplified in FIG. 4F. Such iPS cells, called “checkpoint-competent” pluripotent iPS, would be then advantageous in cell therapy use since unlike currently-used iPSs their teratogenic abilities are largely reduced.

The invention also relates to the use of an inhibitor of the activity and/or of the expression of the Dub3 protein or a variant thereof, said protein comprising the amino acid sequence as set forth in SEQ ID NO: 1, or any variant thereof having at least 43% identity with said amino acid sequence SEQ ID NO: 1, and having ubiquitin hydrolase activity, for inducing the spontaneous differentiation of totipotent or pluripotent cells.

The inventors have made the unexpected observation that inhibition of Dub3 activity and/or expression induce a spontaneous differentiation of totipotent or pluripotent cells.

Inhibitors that can be used are those as mentioned above.

The invention relates to the use of Dub3 protein, said protein comprising the amino acid sequence as set forth in SEQ ID NO: 1, or any variant thereof having at least 43% identity with said amino acid sequence SEQ ID NO: 1 and having ubiquitin hydrolase activity, for determining the differentiation state of cells belonging in a population of cells.

The inventors have also made the unexpected observation that Dub3 protein is rapidly repressed during differentiation process (Dub3 expression is switch off during the differentiation process). Indeed, as shown in examples, Dub3 protein levels dropped massively very early during differentiation, much earlier than Oct4.

Thus, since Oct4 is to date the most commonly used differentiation marker used to determine the differentiation state of cells, the use according to the above definition is advantageous because it gives a more precise status of the cell differentiation state.

The invention also relates to a method for determining the differentiation state of cells belonging in a population of cells, comprising a step of measuring in a cell the presence or amount of Dub3 protein, said protein comprising the amino acid sequence as set forth in SEQ ID NO: 1, or any variant thereof having at least 43% identity with said amino acid sequence SEQ ID NO: 1, and having ubiquitin hydrolase activity, such that:
if Dub3 protein or variant thereof is present, then the cell is a totipotent or a pluripotent cell, and
if Dub3 protein or variant thereof is absent, then the cell is a differentiated cell or a differentiating cell.

By “differentiating cell” it is meant in the invention a cell that morphologically appears to be a totipotent or a pluripotent cell, but harbors molecular signs of differentiation. Molecular signs of differentiation can be, for instance, expression of specific gene such as the endoderm marker Sox7, the neuroectoderm markers Sox1 and Nestin and repression of specific genes, such as the transcription factors of the pluripotency network Nanog, Sox2, Klf4.

Moreover, the invention relates to a method for isolating stem cells from a population of non tumoral cells comprising the determination of the presence or the amount of the Dub3 protein, said protein comprising the amino acid sequence as set forth in SEQ ID NO: 1, or any variant thereof having at least 43% identity with said amino acid sequence SEQ ID NO: 1 and having ubiquitin hydrolase activity, and optionally a step of isolating cells expressing said Dub3 protein.

By using common technics known by the skilled person, such as flow cytometry, and immunological material (i.e. appropriate antibodies directed against Dub3 protein or variant thereof), it is possible to specifically label cells expressing said Dub3 protein, and therefore isolate them from other cells that do not express Dub3 protein or variant thereof.

The invention also relates to a composition comprising
Dub3 protein, said protein comprising the amino acid sequence as set forth in SEQ ID NO: 1, or any variant thereof having at least 43% identity with said amino acid sequence SEQ ID NO: 1 and having ubiquitin hydrolase activity, or
a nucleic acid molecule coding for said protein or said variant thereof, or
an inhibitor of the activity, i.e. the ubiquitin hydrolase activity and/or of the expression of said protein or said variant thereof,

for its use for the treatment of therapy-resistant tumors, or cancers.

Properties of the small group of cancer cells called tumor-initiating or cancer stem cells (CSCs) involved in drug resistance and relapse of cancers can significantly affect tumor therapy. Importantly, tumor drug resistance seems to be closely related to many intrinsic or acquired properties of CSCs, such as quiescence, specific morphology, DNA repair ability and overexpression of antiapoptotic proteins, drug efflux transporters and detoxifying enzymes. The specific microenvironment (niche) and hypoxic stability provide additional protection against anticancer therapy for CSCs. Thus, CSC-focused therapy is destined to form the core of any effective anticancer strategy.

Thus the inventors, intended to solve the problem of the resistance of cancers, propose a new pharmaceutical composition for this purpose.

In one aspect, a composition comprising Dub3 protein, or variant thereof as defined above, or a nucleic acid molecule coding such protein or variant would induce differentiation process in cancer stem cells, rendering such cells susceptible to the therapy adapted to the differentiated cancer cells. In particular embodiment, cancer stem cells expressing the Dub3 protein, or variant thereof, die by apoptosis because they ectopically express Dub3 protein.

In another aspect, a composition comprising an inhibitor or the activity or of the expression of Dub3 protein or a variant thereof would induce spontaneous differentiation of cancer stem cells, rendering such cells susceptible to the therapy adapted to the differentiated cancer cells.

Therefore, the composition according to the invention allows to treat specific types of cancer that are resistant to conventional cancer therapies, such as chemotherapies.

The invention also relates to a method for treating therapy-resistant tumors or cancers, comprising the administration to a patient in a need thereof of an effective amount of a composition comprising:
Dub3 protein, said protein comprising the amino acid sequence as set forth in SEQ ID NO: 1, or any variant thereof having at least 43% identity with said amino acid sequence SEQ ID NO: 1 and having ubiquitin hydrolase activity, or
a nucleic acid molecule coding for said protein or said variant thereof, or
an inhibitor of the activity, i.e. the ubiquitin hydrolase activity and/or of the expression of said protein or said variant thereof.

Advantageously, the invention relates to a composition for its use as defined above, or a method as defined above, comprising an inhibitor of the activity, i.e. the ubiquitin hydrolase activity, and/or of the expression of said Dub3 protein, said inhibitor being chosen among siRNA, miRNA, shRNA, RNA antisense, DNA antisense, antibodies or chemical compounds.

Antibody obtained from the animal immunization by the peptide consisting of the amino acid sequence as set forth in SEQ ID NO: 39.

Compound of formula I, as defined above, is also advantageous.

More advantageously, the invention relates to a composition for its use as defined above, or a method as defined above, wherein said inhibitor is a siRNA comprising of the following amino acid sequence as set forth in SEQ ID NO: 41 or SEQ ID NO:42.

The siRNA of SEQ ID NO: 42 is 5′-UAGCACACAUCUUACAGCC-3′.

Thus, most advantageous siRNA according to the invention is a siRNA comprising a sense strand comprising or consisting in SEQ ID NO: 41 and its complementary sequence, or antisense strand, comprising or consisting of SEQ ID NO: 42.

The above siRNA can also be modified by addition of compounds stabilizing siRNA structure. For instance, the above siRNA contain, in their 3′-end a dinucleotide: a dithymidine (TT).

In one another advantageous embodiment, the invention relates to a composition for its use as defined above, wherein said shRNA comprises or consists of a nucleic acid molecule comprising or being constituted by the sequence SEQ ID NO: 41 followed by the sequence SEQ ID NO: 42, the 3′-end of SEQ ID NO: 41 being linked to the 5′-end of SEQ ID NO: 42 by a linker. The linker according to the invention can be chosen among the following linkers

1)
UUCAAGAGA (Brummelkamp, T.R., 2002 Science.296
(5567):550-3),
2)
AAGUUCUCU (Promega),
3)
UUUGUGUAG (Scherr, M., Curr Med Chem. 2003
February; 10(3):245-56.),
4)
(SEQ ID NO: 43)
CUUCCUGUCA (Schwarz D. S., 2003 Cell. 115(2):
199-208.),
and
5)
CUCGAG.

Nucleic acid molecules coding said shRNA (i.e. DNA coding shRNA) are encompassed by the present invention.

The invention relates to the use of
the Dub3 protein, said protein comprising the amino acid sequence as set forth in SEQ ID NO: 1, or any variant thereof having at least 43% identity with said amino acid sequence SEQ ID NO: 1, or
a nucleic acid molecule coding for said protein or said variant thereof,

for inducing cell death of differentiating stem cells, totipotent cells and/or pluripotent stem cells, preferably in vitro.

As mentioned in the example section, the inventors have shown that enforced expression of Dub3 protein, or variant thereof as defined above, induce both differentiation process in stem cells (or totipotent or pluripotent cells), and cell death by apoptosis.

The invention relates to the a method for inducing cell death of totipotent and or pluripotent stem cells, comprising the administration to said cells an effective amount of:
the Dub3 protein, said protein comprising the amino acid sequence as set forth in SEQ ID NO: 1, or any variant thereof having at least 43% identity with said amino acid sequence SEQ ID NO: 1, or
a nucleic acid molecule coding for said protein or said variant thereof.

The invention will be better understood from the following examples and taking account of the following figures.

### BRIEF DESCRIPTION OF THE DRAWINGS

FIGS. 1A-I show that DNA damage in G1 induces transient cell cycle arrest in early S-phase and not at the G1/S transition

FIG. 1A represents a flow cytometry analysis of DNA content of ES cells treated with various doses of UV. Cell cycle profile of asynchronously growing ES cells exposed to increasing dose of UV-light (0, 2, 4, 6 or 10 J/m2—Z-axis). Cells were collected 6 hours after UV-irradiation for FACS analysis. X axis represents the cell number, and y axis represents the DNA content measured by Propidium Iodide fluorescence.

FIG. 1B represents a flow cytometry analysis of DNA content of ES cells treated with UV in time. Cell cycle profile of asynchronously growing ES cells exposed to increasing dose of UV-light (0, 2, 4, 6 or 10 J/m2). Asynchronously growing ES cells were exposed to 6 J/m2 UV-irradiation and collected for FACS analysis at indicated time points (0, 2, 4 or 6 hours; Z-axis). X axis represents the cell number, and y axis represents the DNA content measured by Propidium Iodide fluorescence.

FIG. 1C is a photography showing the fluorescence detection of DNA content using DAPI in NIH-3t3 cell lines. Scale bar represents 10 μm.

FIG. 1D is a photography showing the fluorescence detection of DNA content using DAPI in ES cells. Scale bar represents 10 μm.

FIG. 1E is a photography showing the immunofluorescence detection of Oct4 protein using specific antibody in NIH-3t3 cell lines. Scale bar represents 10 μm.

FIG. 1F is a photography showing the immunofluorescence detection of Oct4 protein using specific antibody in ES cells. Scale bar represents 10 μm.

FIG. 1G represents a western blot showing the expression of Cyclin A (#1), Histone H3 (#2), γH2AX (#3), DNA polymerase α (#4) and Cdc45 (#5) proteins into soluble (a.) and insoluble (chromatine-bound; b.) fractions of ES cells released from nocodazole arrest untreated or UV-irradiated in G1 (2 hours after release) collected at indicated time points. t: time.

FIG. 1H is a histogram showing the qPCR quantification of Cyclin mRNA normalised to multiple reference genes from ES cells released from nocodazole arrest mock or UV-irradiated in G1 and collected at indicated time points. Dotted line represents levels in G1. Data are expressed as mean±SD (error bars) of multiple observations.

FIG. 1I is a histogram showing the qPCR quantification of Cyclin A2 mRNA normalised to multiple reference genes from ES cells released from nocodazole arrest mock or UV-irradiated in G1 and collected at indicated time points. Dotted line represents levels in G1. Data are expressed as mean±SD (error bars) of multiple observations.

FIGS. 2A-F show that DNA damage in G1 induces transient ES cell cycle arrest in early S-phase and not at the G1/S transition.

FIG. 2A is a schematic overview of the experimental design. Arrows indicate time points at which cells were collected.

FIG. 2B represents a FACS analysis of ES cells released from nocodazole arrest, mock. Analysis of total DNA content stained by propidium iodide at indicated time points.

FIG. 2C represents a FACS analysis of ES cells released from nocodazole arrest, exposed to 6 J/m2 UV light in G1. Analysis of total DNA content stained by propidium iodide at indicated time points.

FIG. 2D represents a FACS analysis of kinetics of S phase entry of synchronised ES cells, mock and UV-irradiated (6 J/m2) in G1. Cell cycle distribution was measured by BrdU incorporation followed by FACS analysis.

FIG. 2E is a curve that summarize FIG. 2D. X-axis represents time in hours, and Y-axis represents the percentage of BrdU positive cells. Curve with black circles represents untreated cells and curve with open squares represents UV-treated cells.

FIG. 2F shows representative FACS analysis of S-phase entry by analysis of BrdU immunoreactivity of ES and NIH-3t3 cells exposed respectively to 6 and 10 J/m2 UV light in G1. Box indicates region were differences in total events was observed. Mean fluorescence intensity of BrdU-positive cells is shown.

FIGS. 3A-F show that p53 is transcriptionally active in ES cells upon DNA damage.

FIG. 3A represents a western blot showing the expression of MCM2 (#1), Chk1(#2), p53^{S15P} (#3), γH2AX (#4) and Histone H3 (#5) in subcellular fractions of ES cells UV-irradiated and collected at indicated time points (hours post UV treatment). Cells were lysed and fractionated into soluble (b.) and insoluble (chromatin-bound; a.) fractions.

FIG. 3B is a histogram showing the relative luciferase activity (firefly/renilla) of ES cells transfected with pG13-luc promoter (containing 13×p53 response elements) untreated (−) or UV-irradiated (+). Bars represent the mean±SD of triplicate observations.

FIG. 3C is a histogram showing the relative luciferase activity (firefly/renilla) of ES cells transfected with p21-luc (white bars) and p21-ΔREp53-luc (lacking p53 response element) (black bars) untreated (−) or UV-irradiated (+). Bars represent the mean±SD of triplicate observations.

FIG. 3D is a histogram showing the relative mRNA expression, measured by qPCR, of p53 gene in Wild-type (white bars) and p53 knockout (n.d.: not determined)) ES cells. ES cells were UV-irradiated and collected at indicated time points (X-axis: time after UV in hours). Bars represent the mean±SD of triplicate observations.

FIG. 3E is a histogram showing the relative mRNA expression, measured by qPCR, of p21 gene in Wild-type (white bars) and p53 knockout (black bars) ES cells. ES cells were UV-irradiated and collected at indicated time points (X-axis: time after UV in hours). Bars represent the mean±SD of triplicate observations.

FIG. 3F is a histogram showing the relative mRNA expression, measured by qPCR, of Mdm2 gene in Wild-type (white bars) and p53 knockout (black bars) ES cells. ES cells were UV-irradiated and collected at indicated time points (X-axis: time after UV in hours). Bars represent the mean±SD of triplicate observations.

FIGS. 4A-F show the persistence of Cdc25A upon DNA damage in G1 sustains G1/S checkpoint bypass in ES cells.

FIG. 4A is a western blot showing expression level of Cdc25A (#1, dark exposure and #2 light exposure), Cdk2 (#3) and β-actin (#4; as control) in asynchronously growing ES (b.) and NIH-3t3 (a.) cells exposed to 10 J/m2 of UV-light and collected at the indicated times (hours post UV).

FIG. 4B is a western blot showing expression level of Cdc25A (#1, dark exposure and #2 light exposure), H3^{S10P} (#3), H3 (#4) and β-actin (#5; as control) in ES (a.) and NIH-3t3 (b.) cells synchronized in G1 and passing through S phase. ES cells were synchronized by nocodazole and collected upon release at indicated time points (release in hours). NIH-3t3 cells were synchronized by confluence, released and collected at 6 hours (G1) and 18 hours (S) after release. To observe posttranslational modifications (PTM; asterisk) of Cdc25A, dark exposure is shown.

FIG. 4C is a western blot of Flag-immunoprecipitated, ectopically expressed Flag-Cdc25A cotransfected with HA-ubiquitin in ES (a.) and NIH-3t3 cells (b.) after MG132 treatment for 1 hour. Presence of Cdc25A (#2, dark exposure and #3 light exposure) and HA (#1) is shown. Immunoglobulins (#4) are also shown.

FIG. 4D is a western blot showing the rapid Cdc25A destruction upon DNA damage is Chk1-dependent in ES cells. Cells were UV-irradiated and incubated with cycloheximide (Cx) in absence or presence of Chk1 inhibitor SB218078, collected at the indicated times (min) and analyzed by western blotting. Cdc25A expression (#1) and β-actin (#2; as control) are shown.

FIG. 4E is a western blot showing the downregulation of Cdc25A expression by RNAi resulting in increased inhibitory CDK2Tyr15 phosphorylation upon DNA damage in G1. Control (a.) and Cdc25A (b.) RNAi-transfected cells were released from nocodazole and exposed (+) to UV-light in G1. Samples were collected at the indicated times and analyzed by western blotting with the indicated antibodies: Cdc25A (#1), Cdk2^{Y15P} (#2), Cyclin B1 (#3), Chk1^{S345P} (#4), Chk1 (#5) and β-actin (#6; as control).

FIG. 4F is a histogram showing Cdc25A downregulation in G1 delay upon DNA damage. Control (a.) and Cdc25A (b.) RNAi-transfected cells were released from nocodazole and exposed to UV light in G1 (t=2) and collected 2 hours (t=4) after UV-(+) or mock-irradiation (−). Prior to collection cells were pulse-labelled with BrdU. Fraction (expressed as %) of diploid BrdU negative cells is plotted (data are represented as mean±SD). Statistical differences is indicated with a single asterisk (*) for P<0.05. Y-axis represents the percentage of cells in G1.

FIGS. 5A-H show that persistent Cdc25A phosphatase upon DNA damage in G1 inhibits G1/S checkpoint in ES cells.

FIG. 5A is a histogram showing the quantification of western blotting signals shown in FIG. 4A Western blot signals (lane 1 (black bar) and lane 7(white bar)) of Cdc25A (dark exposure) were quantified by densitometry scanning and expressed as relative optical density (ROD) compared to β-actin signal as loading control (Y-axis).

FIG. 5B is a histogram showing the quantification of western blotting signals shown in FIG. 4B. Western blot signals of Cdc25A were quantified by densitometry scanning and expressed as relative optical density (ROD) compared to β-actin signal as loading control (Y-axis). Black bars represent NIT-3t3 cells and whit bars represent ES cells.

FIG. 5C is FACS analysis of asynchronously growing ES cells treated with increasing concentration of Roscovitine (in μM; Z-axis). Roscovitine is a potent and selective inhibitor of cyclin-dependent kinases, dependent lengthening of the G1 phase of ES cells. X-axis represents DNA content (expressed in propidium iodide fluorescence) and Y-axis represents the number of cells.

FIG. 5D is a western blot showing the Cdk2 phosphorylation status (Y15P) during an unperturbed cell cycle. ES cells were released from nocodazole arrest and collected in G1 and S-phase at indicated time points. Proteins Cdc25A (#1), Wee1 (#2), Cdk2^{Y15P} (#3), Cdk2 (#4), Cyclin A (#5), H3^{S10P} (#6), H3 (#7) and β-actin (#8; as control) were detected by western blotting.

FIG. 5E is a schematic representation of the regulation of phosphorylation on Cdk2 by Wee1 and Cdc25A. Western blot signals of FIG. 5D were quantified by densitometry scanning and expressed as relative optical density (ROD) compared to β-actin signal as loading control. Right X-axis represents the Cdc25A and Wee1 protein levels, relative to β-actin, and left X-axis represents the Cdk2^{Y15P} expression level. Curve with black circles represents Cdc25A expression level, curve with triangle represents Cdk2^{Y15P} expression level and curve with crosses represents the Wee1 expression level. Y-axis represents he time in hours after release.

FIG. 5F is a histogram representing the qPCR quantification of Cdc25A mRNA normalized to multiple reference genes expressed as percentage of control. ES cells were transfected with control (a.) RNAi or Cdc25A (b.) RNAi sequences. Bars represent the mean±SD of multiple observations.

FIG. 5G is a western blot analysis of ES cells transfected with control (a.) or Cdc25A (b.) RNAi sequences. The expression if Cdc25A of Cdc25A (#1, dark exposure and #2 light exposure), and β-actin (#2; as control) is represented.

FIG. 5H is a histogram showing the quantification of western blotting signals shown in FIG. 4E. Western blot signals of FIG. 4E were quantified by densitometry scanning and expressed as relative optical density (ROD) compared to Chk1 signal as loading control. Black bars represent cells treated with Cdc25A RNAi (a.) and white bars represent cells treated with control RNAi (b.).

FIGS. 6A-J shows that elevated deubiquitylating enzyme Dub3 in ES cells results in Cdc25A abundance.

FIG. 6A shows a representative western blot signal used for determination of Cdc25A turnover rate in the presence of cycloheximide (Cx) during the indicated times (min) in NIH-3t3 cells. Cells were collected at indicated time points. Expresion of Cdc25A (#1) and β-actin (#2; as control) are represented.

FIG. 6B shows a representative western blot signal used for determination of Cdc25A turnover rate in the presence of cycloheximide (Cx) during the indicated times (min) in ES cells. Cells were collected at indicated time points. Expresion of Cdc25A (#1) and β-actin (#2; as control) are represented.

FIG. 6C is a graph showing Cdc25A turnover rate in the presence of cycloheximide (Cx) in ES and NIH-3t3 cells. Western blot signals of Cdc25A were quantified by densitometry scanning and expressed as relative optical density (ROD) compared to β-actin signal as loading control. Signal in untreated cells were set at 100% and half-life (t1/2) of Cdc25A was determined (data are represented as mean±SD). Curve with black circles represents ES cells, and curve with white squares represents NIH-3t3 cells. Y-axis represents Cdc25A protein levels expressed in percent and X-axis represents time in min.

FIG. 6D shows that overexpression of Dub3 increases Cdc25A abundance. NIH-3t3 cells were transduced with empty vector (a.) or pLPC encoding Myc6-Dub3 (b.). After puromycin selection cells were collected and processed for western blot analysis. Proteins were detected with myc (#1), Chk1 (#2), Cdc25A (#3) and β-actin (#4, as control) antibodies.

FIG. 6E is a western blot showing Cdc25A degradation upon DNA damage in NIH-3t3 cells expressing empty vector (a.) or pLPC encoding Myc6-Dub3 (b.). Cells were collected at indicated time points (min post UV treatment) and analyzed by western blotting. Expression of Cdc25A (#1), Myc (#2), Chk1 (#3), Chk1^{S345P} (#4) and β-actin (#4, as control) is indicated.

FIG. 6F is a histogram showing qPCR quantification of β-TrCP normalised to multiple reference genes expressed as percentage of control. ES cells were transfected with control (Luc), β-TrCP (1), Cdh1 (2) or Dub3 (3) RNAi sequences and collected 48 hours after transfection. Bars represent the mean±SD of triplicate observations.

FIG. 6G is a histogram showing qPCR quantification of Dub3 normalised to multiple reference genes expressed as percentage of control. ES cells were transfected with control (Luc), β-TrCP (1), Cdh1 (2) or Dub3 (3) RNAi sequences and collected 48 hours after transfection. Bars represent the mean±SD of triplicate observations.

FIG. 6H is a histogram showing qPCR quantification of Cdh1 normalised to multiple reference genes expressed as percentage of control. ES cells were transfected with control (Luc), β-TrCP (1), Cdh1 (2) or Dub3 (3) RNAi sequences and collected 48 hours after transfection. Bars represent the mean±SD of triplicate observations.

FIG. 6I is a histogram showing qPCR quantification of Cdc25A normalised to multiple reference genes expressed as percentage of control. ES cells were transfected with control (Luc), β-TrCP (1), Cdh1 (2) or Dub3 (3) RNAi sequences and collected 48 hours after transfection. Bars represent the mean±SD of triplicate observations.

FIG. 6J is a western blot analysis of Cdc25A protein expression in Luciferase (a.), β-TrCP (b.) and Cdh1 (c.) RNAi-transfected cells. Expression of Cdc25A (#1) and β-actin (#2) is shown.

FIGS. 7A-F show that elevated deubiquitylase Dub3 in ES cells increases Cdc25A abundance.

FIG. 7A is a histogram showing the qPCR quantification of Oct4 (1), Cdc25A (2), Cdh1 (3), β-TrCP (4) and Dub3 (5) mRNA normalized to multiple reference genes in ES (white bars) and NIH-3t3 (black bars) cells. Data are expressed as mean±SD (error bars) of multiple observations. Statistical differences is indicated with an asterisk P<0.05. Left Y-axis represents the Oct4 mRNA expression and right Y-axis represent mRNA expression of the three other genes.

FIG. 7B is a histogram showing the qPCR quantification of Dub3 mRNA normalised to multiple reference genes. ES cells were transfected with control (1), Dub3 (2) or Cdc25A (3) RNAi sequences.

FIG. 7C is a histogram showing the qPCR quantification of Cdc25A mRNA normalised to multiple reference genes. ES cells were transfected with control (1), Dub3 (2) or Cdc25A (3) RNAi sequences.

FIG. 7D shows a Western blot analysis of ES cells transfected with Dub3 (column 1), (column 3) Cdc25A or control (column 2) RNAi sequences. Expression of CDC25A (#1), Cdc25C (#2) and β-actin (#3, as control) is represented.

FIG. 7E represents nuclei of ES cells stained with DAPI.

FIG. 7F represents cells indicating cellular localisation of pcDNA3-eGFP-Dub3 in ES cells. Scale bar represents 10 μM.

FIGS. 8A-G show that Dub3 is a target gene of the orphan receptor Esrrb.

FIG. 8A is a schematic overview of the Dub3 proximal promoter in mouse (6 kb). Esrrb (shaded boxes) and Sox2 (black boxes) consensus binding sites (RE) are indicated.

FIG. 8B is a histogram representing qPCR quantification of Esrrb (1), Dub3 (2) and Nanog (3) mRNA normalised to multiple reference genes expressed as % of control. ES cells were transfected with control (Crtl) RNAi (white bars) or Esrrb specific RNAi sequence (black bars). Data are expressed as mean±SD (error bars) of multiple observations. Statistical differences is indicated with a single asterisk (*) for P<0.05, not significant is indicated as (ns).

FIG. 8C is a histogram representing qPCR quantification of endogenous Esrrb (a.) or Dub3 (b.) expression in ES cells transfected with empty vector (white bars), Esrrb (black bars) or Esrrb-ΔCter (hatched bars) expressing plasmids. Data are expressed as mean±SD (error bars) of multiple observations. Statistical differences is indicated with a single asterisk (*) for P<0.05.

FIG. 8D is a histogram representing ChIP of Esrrb on Dub3 promoter. Primer pair location along the 6 kb proximal promoter (FIG. 8A) for scanning of Dub3 promoter for Esrrb and Sox2 occupancy. Data are expressed as mean±SD (error bars) of multiple observations. Amylase serves here as a control. Statistical analysis using two-way ANOVA was performed. 1: amylase, 2: pp1, 3: pp2, 4: pp3, 5: pp4, 6: pp5.

FIG. 8E is a histogram representing ChIP of Sox2 on Dub3 promoter. Primer pair location along the 6 kb proximal promoter (FIG. 8A) for scanning of Dub3 promoter for Esrrb and Sox2 occupancy. Data are expressed as mean±SD (error bars) of multiple observations. Amylase serves here as a control. Statistical analysis using two-way ANOVA was performed. 1: amylase, 2: pp1, 3: pp2, 4: pp3, 5: pp4, 6: pp5.

FIG. 8F is a histogram showing Dub3 promoter activity using luciferase assay in CV1 cells. Cells were cotransfected with promoter construct and the indicated genes, and assessed for luciferase activity 48 hours post-transfection. Bars represent the fold induction±SD of multiple observations. Statistical differences is indicated with a single asterisk (*) for P<0.05 and (**) for P<0.001. Black bars represents pGL4.10_5′far and white bars represents pGL4.10_3.2 kb. 1: empty vector, 2: Sox2, 3: Essrb and 4: Δ-Cter.

FIG. 8G is a histogram showing basal transcriptional activity of a 1 kb proximal promoter and a mutated sequence in ES cells. Three mutations were introduced in the Esrrb consensus binding site. TCAAGGTCA was mutated to TCATTTTCA. Data are expressed as mean±SD (error bars) of multiple observations. 1: wt, 2: mutated.

FIGS. 9A-F shows that Dub3 is a target gene of the orphan receptor Esrrb

FIG. 9A is a graph showing qPCR quantification of Cdc25A (curves with black squares) and Dub3 (curves with triangles) mRNA in ES cells treated with increasing concentration of the selective Esrrb and Esrrg agonist DY131 (indicated doses in μM) for 16 hours. Bars represent the fold induction±SD of triplicate observations.

FIG. 9B is a western blot analysis of Cdc25A protein levels in ES cells treated with increasing concentrations (in μM) of the DY131 agonist for 16 hours. Cdc25A (#1) and β-actin (#2, as control) protein expression is represented.

FIG. 9C is a histogram showing qPCR quantification of Esrrb (1) and Dub3 (2) mRNA normalised to multiple reference genes expressed as percentage of control in presence of DY131. ES cells were transfected with control (Crtl) RNAi (white bars) or Esrrb specific RNAi sequence (black bars). Data are expressed as mean±SD (error bars) of multiple observations.

FIG. 9D represents DNA fragments size prior to ChIP analysis. Sonication resulted to DNA fragments smaller than 500 bp. 1: IP input, 2: genomic DNA.

FIG. 9E is a western blot showing the specificity of the Esrrb antibody. Immunoprecipitation of 293T-HEK cells transfected with either empty vector (Ev; #2) or Flag-Esrrb (#1) expression plasmids. Immunoprecipitation was performed in parallel using either Flag or Esrrb (b.) antibody. Both antibodies specifically immunopreciptated Flag-Esrrb protein. a: input. a1: IgGs, a2: Esrrb and a3: β-actin.

FIG. 9E is a western blot analysis of expression levels of CV1 cells transfected with either empty vector (lane 1), Flag-Esrrb (lane 2), Flag-Esrrb-Δ-Cter (lane 3) or Sox2 (lane 4), 48 hours post-transfection. a1: FLAG, a2: Esrrb and a3: β-actin, #1: Esrrb and #2: Δ-Cter.

FIGS. 10A-K show Developmental regulation of Cdc25A protein abundance correlates with Dub3 expression.

FIG. 10A is a phase-contrast photo of ES cells.

FIG. 10B is a phase-contrast photo of N2B27-induced neural conversion of ES cells at day 1.

FIG. 10C is a phase-contrast photo of N2B27-induced neural conversion of ES cells at day 3.

FIG. 10D is a phase-contrast photo of N2B27-induced neural conversion of ES cells at day 7.

FIG. 10E is a phase-contrast photo of N2B27-induced neural conversion of ES cells at neural differentiated state.

FIG. 10F is a graph representing qPCR quantification of Dub3 (curve with triangles), Sox2 (curve with open squares) and Esrrb (curve with inversed triangles) mRNA normalised to multiple reference genes during N2B27-induced neural differentiation. Values represent mean±SD of multiple observations.

FIG. 10G is a graph representing qPCR quantification of Dub3 (curve with triangles), Cdc25A (curve with squares), Cdh1 (curve with circles) and β-TrCP (curve with crosses) mRNA normalised to multiple reference genes during N2B27-induced neural differentiation. Values represent mean±SD of multiple observations.

FIG. 10H is a graph representing qPCR quantification of Dub3 (curve with triangles), USP48 (curve with squares), USP13 (curve with diamonds) and USP29 (curve with inversed triangles) mRNA normalised to multiple reference genes during N2B27-induced neural differentiation. Values represent mean±SD of multiple observations.

FIG. 10I represents a western blot analysis of cell extracts collected throughout differentiation of ES cells into neural stem cells (NSC) immunoblotted Dub3 (#1), Oct4 (#2), Cdc24A (#3), RhoA (#4) Suds3 (#5) and β-actin (#6, as control) antibodies.

FIG. 10J represents a western blot analysis of asynchronously growing ES (a.) and NSC (b.). Cells were exposed to 6 J/m2 UV-light and collected at indicated times. Expression of Cdc25A (#1, dark exposure and #2 light exposure) and β-actin (#3, as control) are represented.

FIG. 10K is a histogram representing the basal transcriptional activity of three different promoter lengths of the Dub3 gene in NIH-3t3 (a.) cells and ES (b.) cells. Data are expressed as mean±SD (error bars) of multiple observations. Black bars: 1 kb, white bars: 1.7 kb and hatched bars: 3.2 kb. X axis represents the Dub3 promoter activity expressed as luciferase fold induction.

FIGS. 11A-J show that developmental regulation of Cdc25A protein abundance correlates with Dub3 expression levels.

FIG. 11A is a graph showing qPCR quantification of pluripotency markers (Oct4: curve with open circles, nanog: curves with black diamonds and Klf4: curve with open squares) during neural conversion of ES cells. Data were normalized to multiple reference genes. Data are expressed as mean±SD (error bars) of multiple observations. Left Y-axis represents Nanog or Klf4 mRNA expression and right Y-axis represents Oct4 mRNA expression.

FIG. 11B is a graph showing qPCR quantification of cell fate specification markers (Nestin: curve with diamonds, Sox7: curve with black squares and Sox1: curve with open squares) during neural conversion of ES cells. Data were normalized to multiple reference genes. Data are expressed as mean±SD (error bars) of multiple observations. Left Y-axis represents Sox7 expression and right Y-axis represents Sox1 or Nestin mRNA expression.

FIG. 11C is an immunofluorescence detection of Nestin at day 1 of N2B27-induced differentiation. Nuclei were counterstained using DAPI. Scale bar 50 μM.

FIG. 11D is an immunofluorescence detection of Nestin at day 6 of N2B27-induced differentiation. Nuclei were counterstained using DAPI. Scale bar 50 μM.

FIG. 11E is a western blot showing specificity of the antibody raised against mouse Dub3. Human 293T cells were transfected with empty vector (EV; lanes 2 and 4) or HA-Dub3 expressing vectors (lanes 3 or 5). Cells were collected 24 hours post transfection and extracts were immunoblotted (IB) using pre-immune (PI; lane 1), Dub3 (lanes 2 and 3) or HA (lanes 4 or 4) antibodies. The Dub3 antibody recognizes a specific polypeptide of 60 kDa in SDS-PAGE (arrow) which is not recognized by the pre-immune serum.

FIG. 11F is a western blot showing the validation of the antibody raised against mouse Dub3. Western blot analysis of ES cells transfected with control (lane 1) or Dub3 (lane 2) RNAi sequences. Cells were collected 48 hours post transfection and extracts were immunoblotted using Dub3 purified antibody (#1) and β-actin (#2).

FIG. 11G is a western blot analysis of Dub3 substrates and other proteins (#1: Oct4, #2: Cdc25A, #3: Cdc25B, #4: Cdc25C, #5: PCNA and #6: β-actin) during neural conversion of N2B27 cells (from D1 to D7).

FIG. 11H is a graph showing qPCR quantification of Suds3, RhoA and Esrr γ during neural conversion of ES cells. Data were normalized to multiple reference genes. Data are expressed as mean±SD (error bars) of multiple observations.

FIG. 11I is a histogram showing qPCR quantification of Nestin, Nanog, Cdc25A and Dub3 mRNA normalized to multiple reference genes in ES and Neural Stem Cells (NSC). Bars represent the mean±SD of multiple observations.

FIG. 11J is a graph showing qPCR quantification of GI cyclin stoechiometry during neural conversion of ES cells. Data were normalised to multiple reference genes. Data are expressed as mean±SD (error bars) of multiple observations. Left Y-axis represents Cyclin D1 expression and right Y-axis represents Cyclin El mRNA expression.

FIGS. 12A-O show that constitutive Dub3 expression leads to massive apoptosis concomitant to differentiation-induced cell cycle remodeling.

FIG. 12A is a fluorescence detection of empty vector (EV)-expressing ES cells labeled by DAPI staining.

FIG. 12B is an immunofluorescence detection of empty vector (EV)-expressing ES cells. eGFP expression is shown.

FIG. 12C is a fluorescence detection of eGFP-Dub3-expressing ES cells labeled by DAPI staining.

FIG. 12D is an immunofluorescence detection of eGFP-Dub3-expressing ES cells cells. eGFP expression is shown. All ES cells express eGFP-Dub3 at comparable levels.

FIG. 12E is a phase-contrast photo of empty vector (EV) ES cells after LIF removal at the indicated day 0 of differentiation.

FIG. 12F is a phase-contrast photo of empty vector (EV) ES cells after LIF removal at the indicated day 2 of differentiation.

FIG. 12G is a phase-contrast photo of empty vector (EV) ES cells after LIF removal at the indicated day 4 of differentiation.

FIG. 12H is a phase-contrast photo of eGFP-Dub3-expressing ES cells after LIF removal at the indicated day 0 of differentiation.

FIG. 12I is a phase-contrast photo of eGFP-Dub3-expressing ES cells after LIF removal at the indicated day 2 of differentiation. Arrow indicates detached cells with apoptotic morphology.

FIG. 12J is a phase-contrast photo of eGFP-Dub3-expressing ES cells after LIF removal at the indicated day 4 of differentiation. Arrows indicate detached cells with apoptotic morphology.

FIG. 12K shows a western blot of cell extracts prepared every day after LIF withdrawal from empty vector (a.) or eGFP-Dub3-expressing ES cells (b.). (*) indicates a non-specific band. High caspase 3 activities in eGFP-Dub3 expressing cells indicate apoptosis. Expression of GFP-Dub3 (#1), Oct4 (#2), active caspase 3 (#3) and MCM2 (#4) is represented.

FIG. 12L shows differentiation-induced cell cycle remodelling. Cells were collected at the indicated days and analyzed by FACS following propidium iodide staining. Cell death is illustrated by cells with subdiploid DNA content (Sub-G1). Upper lanes represents empty vector expressing cells and lower lane represents eGFP-Dub3 expressing cells. First column represents day 0 of differentiation, second column represents day 1 of differentiation, third column represents day 2 of differentiation, fourth column represents day 3 of differentiation and fifth column represents day 4 of differentiation.

FIG. 12M is a histogram showing a clonogenic assay of ES cells upon prolonged control (1), Dub3 (2) or Cdc25a (3) targeting RNAi sequence. Cells were plated at clonal density in LIF-containing serum and stained for AP after 7 days. Columns show the percentage of alkaline phosphatase (AP) positive (dark grey) or negative (light grey) colonies. At least 150 colonies were scored.

FIG. 12N is a representative picture of cells transfected with control targeting RNAi sequence and assayed for AP activity.

FIG. 12O is a representative picture of cells transfected with Dub3 targeting RNAi sequence and assayed for AP activity.

FIGS. 13A-AF shows that constitutive Dub3 expression leads to massive apoptosis concomitant to differentiation-induced cell cycle remodeling.

FIG. 13A shows cell cycle distribution and BrdU incorporation of empty vector expressing ES cells analyzed by FACS.

FIG. 13B shows cell cycle distribution and BrdU incorporation of eGFP-Dub3 expressing ES cells analyzed by FACS.

FIG. 13C is an immunofluorescence detection of DNA during LIF withdrawal in empty vector expressing ES cells at day 0 of differentiation.

FIG. 13D is an immunofluorescence detection of active caspase 3 LIF withdrawal in empty vector expressing ES cells at day 0 of differentiation.

FIG. 13E is an immunofluorescence detection of DNA during LIF withdrawal in eGFP-Dub3 expressing ES cells at day 0 of differentiation.

FIG. 13F is an immunofluorescence detection of active caspase 3 LIF withdrawal eGFP-Dub3 expressing ES cells at day 0 of differentiation.

FIG. 13G is an immunofluorescence detection of DNA during LIF withdrawal in empty vector expressing ES cells at day 1 of differentiation.

FIG. 13H is an immunofluorescence detection of active caspase 3 LIF withdrawal in empty vector expressing ES cells at day 1 of differentiation.

FIG. 13I is an immunofluorescence detection of DNA during LIF withdrawal in eGFP-Dub3 expressing ES cells at day 1 of differentiation.

FIG. 13J is an immunofluorescence detection of active caspase 3 LIF withdrawal eGFP-Dub3 expressing ES cells at day 1 of differentiation.

FIG. 13K is an immunofluorescence detection of DNA during LIF withdrawal in empty vector expressing ES cells at day 2 of differentiation.

FIG. 13L is an immunofluorescence detection of active caspase 3 LIF withdrawal in empty vector expressing ES cells at day 2 of differentiation.

FIG. 13M is an immunofluorescence detection of DNA during LIF withdrawal in eGFP-Dub3 expressing ES cells at day 2 of differentiation.

FIG. 13N is an immunofluorescence detection of active caspase 3 LIF withdrawal eGFP-Dub3 expressing ES cells at day 2 of differentiation.

FIG. 13O is an immunofluorescence detection of DNA during LIF withdrawal in empty vector expressing ES cells at day 3 of differentiation.

FIG. 13P is an immunofluorescence detection of active caspase 3 LIF withdrawal in empty vector expressing ES cells at day 3 of differentiation.

FIG. 13Q is an immunofluorescence detection of DNA during LIF withdrawal in eGFP-Dub3 expressing ES cells at day 3 of differentiation.

FIG. 13R is an immunofluorescence detection of active caspase 3 LIF withdrawal eGFP-Dub3 expressing ES cells at day 3 of differentiation.

FIG. 13S is an immunofluorescence detection of DNA during LIF withdrawal in empty vector expressing ES cells at day 4 of differentiation.

FIG. 13T is an immunofluorescence detection of active caspase 3 LIF withdrawal in empty vector expressing ES cells at day 4 of differentiation.

FIG. 13U is an immunofluorescence detection of DNA during LIF withdrawal in eGFP-Dub3 expressing ES cells at day 4 of differentiation.

FIG. 13V is an immunofluorescence detection of active caspase 3 LIF withdrawal eGFP-Dub3 expressing ES cells at day 4 of differentiation.

FIG. 13W is a phase contrast photo of empty vector expressing cell-lines.

FIG. 13X is a phase contrast photo of eGFP-Dub3 expressing cell-lines.

FIG. 13Y is a graph showing qPCR quantification of Nanog normalised to multiple reference genes during LIF withdrawal (X-axis, in day). Curve with circles: empty vector, curve with squares: GFP-Dub3 expressing cells.

FIG. 13Z is a graph showing qPCR quantification of Klf4 normalised to multiple reference genes during LIF withdrawal. Curve with circles: empty vector, curve with squares: GFP-Dub3 expressing cells.

FIG. 13AA is a graph showing qPCR quantification of Oct4 normalised to multiple reference genes during LIF withdrawal. Curve with circles: empty vector, curve with squares: GFP-Dub3 expressing cells.

FIG. 13AB is a graph showing qPCR quantification of Rex1 normalised to multiple reference genes during LIF withdrawal. Curve with circles: empty vector, curve with squares: GFP-Dub3 expressing cells.

FIG. 13AC is a graph showing qPCR quantification of Sox7 normalised to multiple reference genes during LIF withdrawal. Curve with circles: empty vector, curve with squares: GFP-Dub3 expressing cells.

FIG. 13AD is a graph showing qPCR quantification of Noxa normalised to multiple reference genes during LIF withdrawal. Curve with circles: empty vector, curve with squares: GFP-Dub3 expressing cells.

FIG. 13AE is a western blot analysis of cell extracts collected every day throughout the N2B27-induced differentiation process of empty vector (b.) or eGFP-Dub3 (a.) expressing ES cells into NSCs. Four days after N2B27-mediated differentiation all eGFP-Dub3 expressing cells were all dead by apoptosis as indicated by high caspase 3 activities. Expression of GFP-Dub3 (#1), Oct4 (#2), active caspase 3 (#3) and MCM2 (#4) is represented.

FIG. 13AE is a western blot analysis of cell extracts collected every day throughout the differentiation process of empty vector or HA-Dub3 expressing ES cells into NSCs. The molecular and cellular phenotype of HA-Dub3 expressing cells was highly comparable to the eGFP-Dub3 expressing cells indicating that the phenotype is independent of the N-terminal tag. Expression of HA-Dub3 (#1), Oct4 (#2), active caspase 3 (#3) and MCM2 (#4) is represented.

### DETAILED DESCRIPTION OF THE INVENTION

### EXAMPLE 1

### Experimental Procedures

### 1—Cell Extracts, Western Blotting and Antibodies

Cells were rinsed once in PBS and then incubated with ice cold lysis buffer (50 mM Tris-HCl pH 7.4, 100 mM NaCl, 50 mM NaF, 5 mM EDTA, 40 mM β-glycero-phosphate, 1% Triton X-100 and protease inhibitors) for 30 min on ice before scraping. Whole cell extracts were clarified by centrifugation at 12000 rcf for 10 min at 4° C. Protein concentration of the clarified lysates was estimated using BCA method (Pierce). Equal amount of protein was used for western blot analysis. All antibodies were incubated overnight at 4° C. in phosphate-buffered saline (PBS) containing 1% BSA and 0.1% Tween (Sigma). Antibodies used from Cell Signaling: Chk1S345P (2341), p53S15P (9284), γH2AX (2577), CDK2Y15P (9111), Myc-Tag (2276); Active caspase 3 (9961); Abcam: DNA polα (ab31777), H3 (ab1791), CDK2 (ab6538), PSTAIR (ab10345), GFP (ab290), MCM2 (ab4461); Suds3 (ab3740) Santa Cruz: Cdc45 (sc-20685), Cdc25A (sc-7389), Chk1 (sc-8408), Cyclin B1 (sc-245), Cdc25C (sc-327), Cdc25B (sc-65504), p21 (sc-6246), RhoA (sc-418); anti-goat IgG-HRP (sc-2020) Sigma: (PC10), β-actin (A1978), Cyclin A (C7410), Anti-Flag M2 (F1804), Oct4 (Chemicon, AB3209), and Millipore, Nestin (Ab353), H3S10P (Millipore 09-797). Wee1 (kindly provided by T. Lorca, CRBM Montpellier).

Mouse Dub3 polyclonal antibodies were raised by immunizing rabbits with a synthetic peptide (NH2-MSPGQLCSQGGR-COOH SEQ ID NO: 39) designed from mouse Dub3 C-terminus, coupled to keyhole limpet hemocyanin (KLH). Antibodies were purified by coupling the Dub3 peptide on HiTrap NHS-activated HP columns (GE Healthcare).

### 2—Cell Culture and Transfection

ES cells (CGR8) were cultured on gelatin-coated dishes in the absence of feeder cells with 1,000 U LIF per ml (Millipore). Cells were grown in a humidified atmosphere of 5% CO2 at 37° C. For transient expression both NIH-3t3 and ES cells were transfected using X-tremeGENE 9 DNA (Roche), and CV1 with JetPEI (Polyplus), according to manufacturer's directions. For infection, retroviral particles were generated by transfecting Platinum-E ecotropic packaging cell line with retroviral expression vector (pLPC) encoding Myc6-Dub3 variants using home-made PEI reagent.

Briefly, ES cells were maintained in Glasgow MEM BHK-21 (GMEM) supplemented with 10% fetal bovine serum, non-essential amino acids, L-glutamine, sodium pyruvate, β-mercapthethanol. NIH-3t3 cells were maintained in Dulbecco's modified eagle's medium (DMEM) supplemented with 10% fetal bovine serum, 2 mM glutamine and antibiotics. The viruses-containing conditioned medium was incubated on exponentially growing NIH-3t3 cells for 24 hours in the presence of polybrene (10 mg/mL). 48 hours post-infection, cells were selected in puromycin (2.5 μg/mL)-containing medium for 8-10 days before use. Reverse transfection of ES cells was performed using INTERFERin (Polyplus) according to manufacturer's directions. Cells were collected 24, 36 or 48 hours after transfection for analysis. The Cdc25A RNAi sequence was:

SEQ ID NO: 40
5′-GAAAUUUCCCUGACGAGAA-3′,

The Dub3 RNAi sequence was:

SEQ ID NO: 41
5′-GGCUGUAAGAUGUGUGCUA-3′

and a Esrrb previously described in Feng et al., 2009, Nat Cell Biol 11, 197-203. RNAi for Cdh1 and β-TrCP knockdown were purchased from Darmacon (SMARTpool) 57371 (Cdh1) and 12234 (β-TrCP).

### 3—Cell Synchronization

ES cells were arrested in prometaphase by nocodazole (Sigma) for 4-8 hours. After mitotic-shake off cells were washed 3 times in ice-cold PBS and dissolved in full ES growth medium. Cells were incubated in a humidified atmosphere of 5% CO2 at 37° C. for 45 minutes and placed at 30° C. for 1 hour to reduce S phase entry. Cells were mock- or UV-irradiated (6 J/m2) and incubated at 37° C. prior collection. To synchronise NIH-3t3 cells in G0 cells were grown to confluence and incubated for 2-3 days. Next, cells were washed, resuspended and split at 30% confluency. Six hours after release, cells were UV-irradiated.

### 4—UV-Induced DNA Damage and Drugs

UVC irradiation at 254 nm was performed with microprocessor-controlled crosslinker (BIO-LINK®) or with a UV-lamp (Hanovia). Cycloheximide and DY131 (GW4716) were from Sigma and Chk1 inhibitor SB218078 from Calbochiem.

### 5—Flow Cytometry

Single-cell suspensions were prepared by trypsinisation and washed once in PBS. Cells were fixed in ice-cold 70% ethanol (−20° C.) and stored at −20° C. overnight. Following RNAse A treatment, total DNA was stained with propidium iodide (25 μg/ml). For BrdU uptake analysis, ES cells and NIH-3t3 cells were grown in the presence of 10 μM BrdU for respectively 10 and 30 minutes. The BrdU content was determined by reaction with a fluorescein isothiocyanate (FITC)-conjugated anti-BrdU antibody (BD Biosciences). Cells were analyzed with a FACScalibur flow cytometer using CellQuestPro software.

### 6—RNA Extraction, Reverse Transcription and Quantitative Real-Time PCR

Total RNA was isolated with TRIzol reagent (Invitrogen). Reverse transcription was carried out with random hexanucleotides (Sigma) and Superscript II First-Strand cDNA synthesis kit (Invitrogen). Quantitative PCRs were performed using Lightcycler SYBR Green I Master mix (Roche) on Lightcycler apparatus (Roche). All primers used were intronspanning (primer sequences available upon request). The relative amount of target cDNA was obtained by normalisation using geometric averaging of multiple internal control genes (ACTB, HPRT, HMBS, GAPDH, SDHA).

### 7—Chromatin Immunoprecipitation

ES cells were formaldehyde cross-linked and sonicated using a Misonix sonicator S-4000. Cells were lysed in ice-cold lysis buffer (Supplemental Information). Primer pairs for promoter scanning (6 kb upstream of transcription start site, TSS) of the Dub3 murine promoter were designed approximately every 1 kb.

Cells were lysed in ice-cold lysis buffer (50 mM Tris-HCl pH 7.4, 100 mM NaCl, 50 mM NaF, 5 mM EDTA, 40 mM β-glycero-phosphate, 1% SDS, 1% Triton X-100 and protease inhibitors) for 30 min on ice. Immuoprecipitation was performed by adding 5 μg Esrrb (Sigma SAB2100715), Sox2 (Bethyl A301-739) or control antibodies (Peprotech 500-P00) to lysates and incubation with rotation overnight at 4° C. BSA and salmon sperm-blocked Protein A-Sepharose (Amersham) beads were added to the lysate.

8—Monolayer Differentiation of ES Cells into Neurectodermal Precursors

ES cells were dissociated and plated in N2B27 medium onto 0.1% gelatine-coated dishes at a density of 1.10⁴ cells/cm². N2B27 medium is a 1:1 mixture of DMEM/F12 (Gibco) supplemented with modified N2 (25 μg/ml insulin, 100 μg/ml apo-transferrin, 6 ng/ml progesterone (Sigma), 16 μg/ml putrescine (Sigma), 30 nM sodium selenite (Sigma), 50 μg/ml bovine serum albumine (Gibco), Neurobasal medium supplemented with B27 (Gibco), β-mercaptoethanol (0.1 mM) and glutamate (0.2 mM) was also added. The medium was replaced every two days until day 7.

9—Isolation and Amplification of NSC Cells from CGR8 ES Cells

ES cells were induced to differentiate into NSC following the protocol described above. At day 6, cells were dissociated in 0.01% Trypsine-EDTA and plated onto Poly-L-Ornithine/Laminin coated dishes in DMEM/N2 medium with 10 ng/ml of both EGF and bFGF (Biosource). For the preparation of Poly-L-Ornithine/Laminin plates, a 0.01% solution of poly-L-ornithine (Sigma) was added to plates for at least 20 min. The solution was removed and plates were washed 3 times with PBS. A 1 μg/ml solution of laminin in PBS (Sigma) was then applied and incubated at 37° C. for at least 3 hrs. Cells can then be cultivated and amplified under these conditions for several subpassages without loosing neural stem cells properties.

10—Establishment of a Monoclonal eGFP-Dub3 Expressing ES Cells

Wild-type ES cells were transfected with pcDNA3-eGFPDub3, plated at clonal density and selected with G418 (Sigma). eGFP-Dub3 positive clones were expanded in continuous presence of G418 and validated by immunofluorescence and western blotting.

### 11—Plasmids

The murine Dub3 gene (Gene ID: 625530) was amplified by PCR and cloned into pLPC-Myc6, pcDNA3-GFP and pcDNA3-HA. All constructs were verified by DNA sequencing. Mouse Esrrb (pSG5FI-mEsrrb) and the C-terminal truncated pSG5FI-mEsrrb-ΔCter were previously described. Genomic sequences of the Dub3 promoter were amplified by PCR and inserted into pGL4.10 vector (Promega) for luciferase activity. pCEP4-Sox2 was a kind gift of F. Poulat (IGH-CNRS).

### 12—Luciferase Assay

ES cells were transfected with following reporter constructs, pG13-luciferase, p21-luciferase and p21-ΔREp53-luc (kindly provided by J. Basbous, IGH, Montpellier). A Renilla luciferase plasmid was cotransfected as an internal control. Cells were harvested 24 hours after transfection and mock or UV-irradiated. Six hours following UV-induced DNA damage, cells were harvested and the luciferase activities of the cell lysates were measured using the Dual-luciferase Reporter Assay system (Promega). The proximal promoter of 1 kb upstream ATG start codon was inserted into pGL4.10 plasmid. Three mutations of the Esrrb consensus binding site (TCAAGGTCA) were introduced by PCR to generate a mutated binding site (TCATTTTCA). All constructs were sequence verified.

### 13—Immunofluorescence Microscopy

For Nestin, Oct4 and active caspase 3 staining staining, cells were fixed in 4% paraformaldehyde and permeabilized with 0.1% Triton X-100. After fixation, cells were blocked in 3% BSA PBS-Tween and incubated overnight with antibody. The slides were mounted using Prolong Gold with DAPI (Invitrogen). For determination of the cellular localisation of Dub3, mouse ES cells were transfected with pcDNA-GFP-Dub3 and directly fixed. All slides were analysed using a Leica DM6000 epifluorescence microscope. Images were acquired using a Coolsnap HQ CCD camera (Photometrics) and the metamorph software (Molecular Devices).

### 14—Subcellular Fractionation Experiments

Chromatin-enriched and soluble fractions were prepared using CSK-extraction procedure. Briefly, pelleted cells were lysed in CSK buffer (10 mM PIPES pH 6.8, 100 mM NaCl, 300 mM sucrose, 1 mM EGTA, 1 mM MgCl₂, 0.5 mM DTT, 1 mM ATP, 0.2% Triton X-100 and protease inhibitors) for 10 min on ice. After centrifugation at 3000 rpm for 3 min at 4° C., the supernatant (Triton-soluble fraction) was recovered and the pellet (Triton-insoluble fraction) was resuspended in CSK buffer and incubated for 10 min on ice. After centrifugation, the pellet (chromatin-enriched fraction) was resuspended in Laemmli Buffer. Equivalent amount of soluble and chromatin fractions were analyzed by immunoblotting.

### 15—Statistical Analysis

Two-way ANOVA or Student t-test were used to evaluate differences between groups using Prism software (GraphPad Software). P<0.05 was considered significant and indicated with *, P<0.001 was indicated with **. 30

### Example 2

### Experimental Results

ES Cells Arrest in Early S Phase upon Induction of DNA Camage in G1

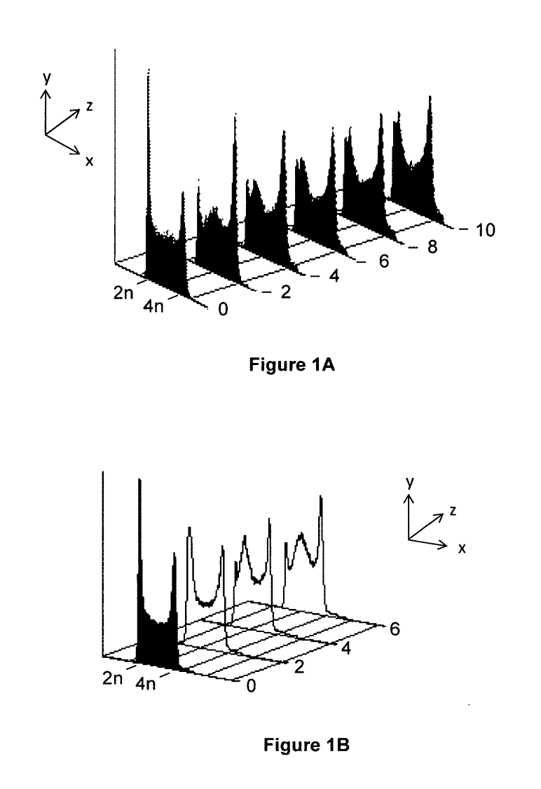

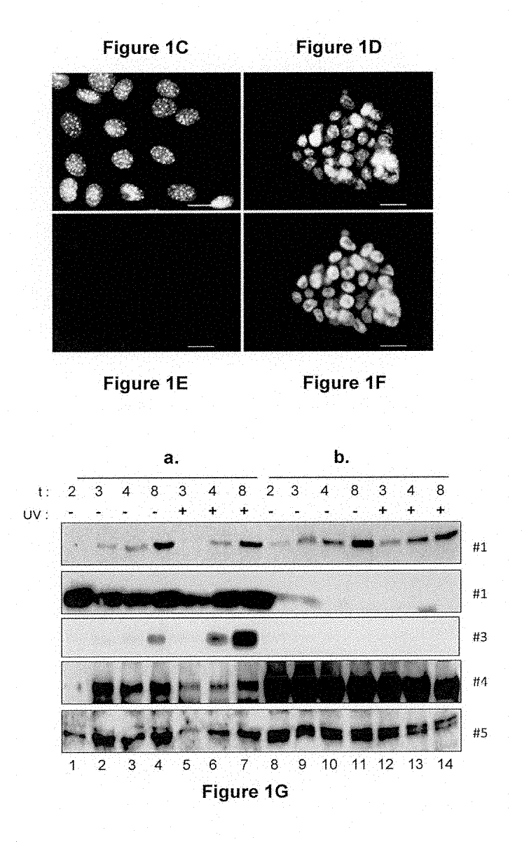

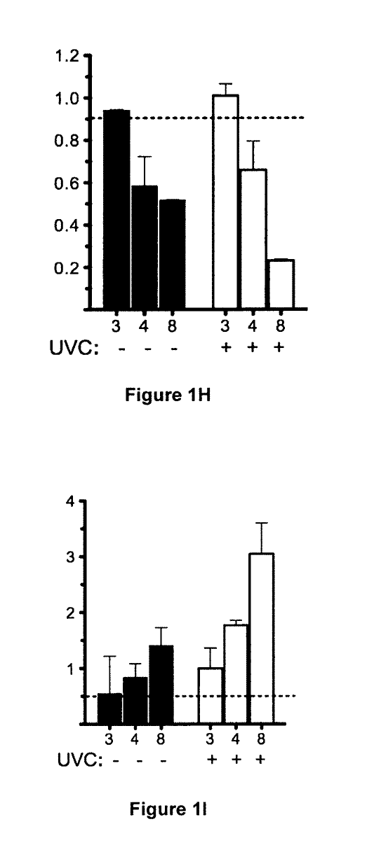

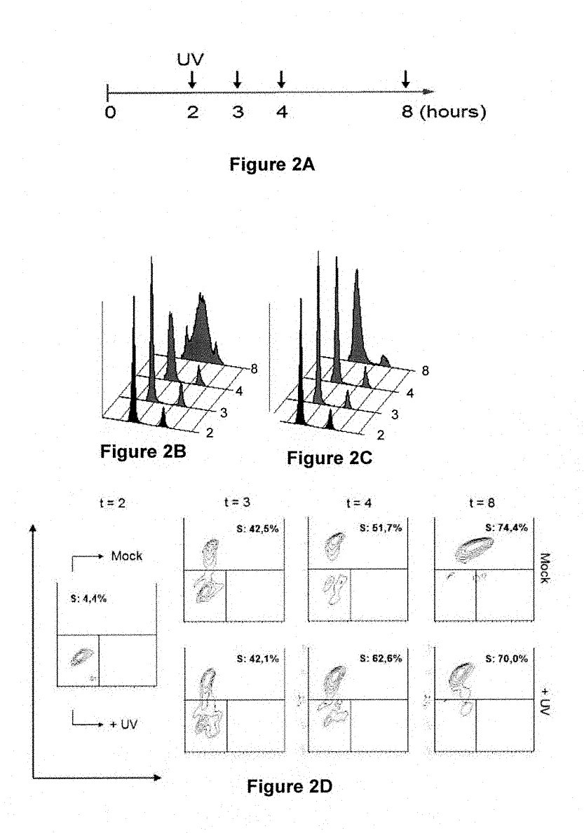

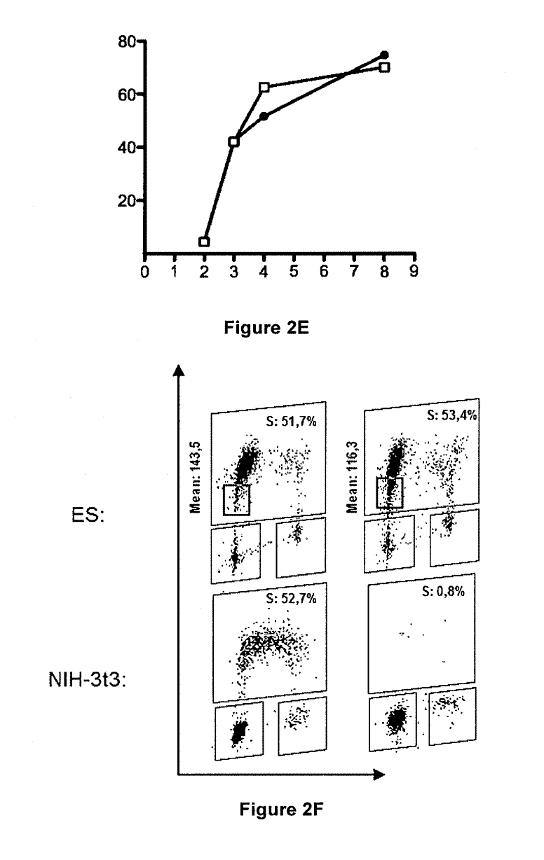

Circumstantial data suggest an impaired G1/S checkpoint in ES cells. The inventors observed that irradiation of ES cells with increasing doses of UV light induced a decrease in the number of G1 cells (FIG. 1A). Time course analysis with a single UV dose (6 J/m2) resulted in cell cycle delay at the G1/S boundary (FIG. 1B, t=2). The inventors pulse-labelled nocodazole synchronized cells with BrdU (a nucleotide analogue) to allow exact distinction between late G1 (BrdU-negative) and early S-phase (BrdU-positive, FIG. 2A). While analysis of total DNA content suggests a G1 arrest (FIG. 2B), analysis of BrdU incorporation revealed that both untreated (Mock) and UV-irradiated cells (+UV) entered S phase with very similar kinetics (FIG. 2C-D). In contrast, synchronized mouse embryonic fibroblasts (NIH-3t3), which are Oct4-negative differentiated cells (FIG. 1C-F), did not progress to S phase after UV irradiation in G1 (FIG. 2E), in line with the presence of a stringent G1/S checkpoint.

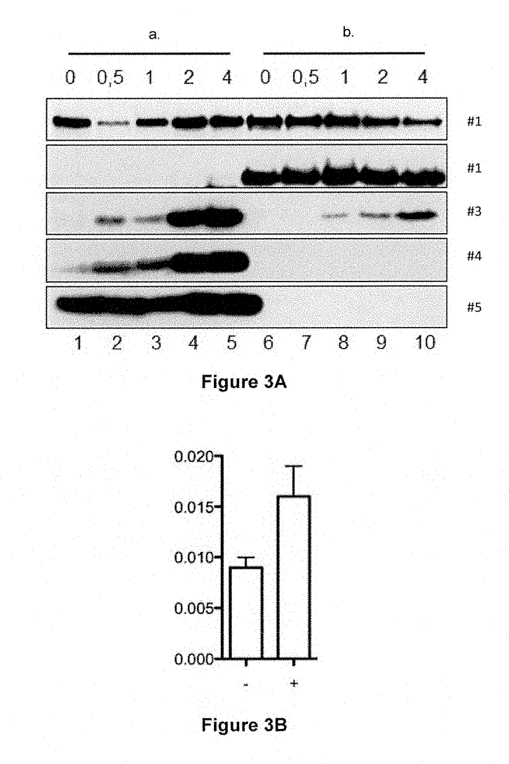

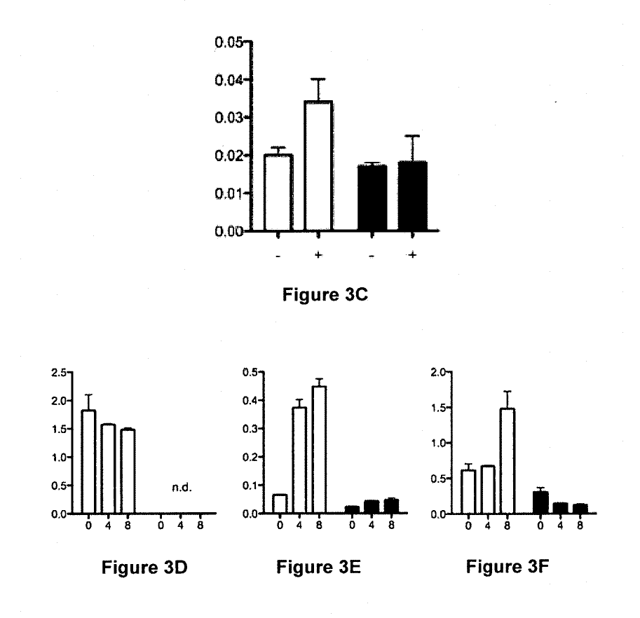

The inventors noticed that upon UV irradiation, BrdU incorporation was slightly reduced compared to mock-irradiated cells, confirmed by calculating the mean fluorescent signal of BrdU-positive cells (FIG. 1E, green boxes), and suggesting DNA synthesis slowdown in very early S phase. Analysis of chromatin-bound proteins shows that recruitment of both Cdc45 and DNA polymerase-a, two replication fork-associated factors, was considerably reduced upon UV irradiation, but not abolished (FIG. 1G, compare lanes 2-4 with 5-7), suggesting activation of the S phase checkpoint preventing late replication origins firing. Consistent with this possibility, phosphorylated H2AX histone variant (γH2AX), an ATR substrate, accumulated onto chromatin. Moreover UV-induced DNA damage did not significantly change the transcriptional program driven by E2F transcription factors required for S phase entry, as monitored by Cyclin A2 and El production (FIG. 1H-I). The inventors also observed UV damage-dependent p53 phosphorylation on chromatin (FIG. 3A), and transactivation (amongst other) of p21 gene expression (FIGS. 3B-D), demonstrating a functional p53 transcriptional response.

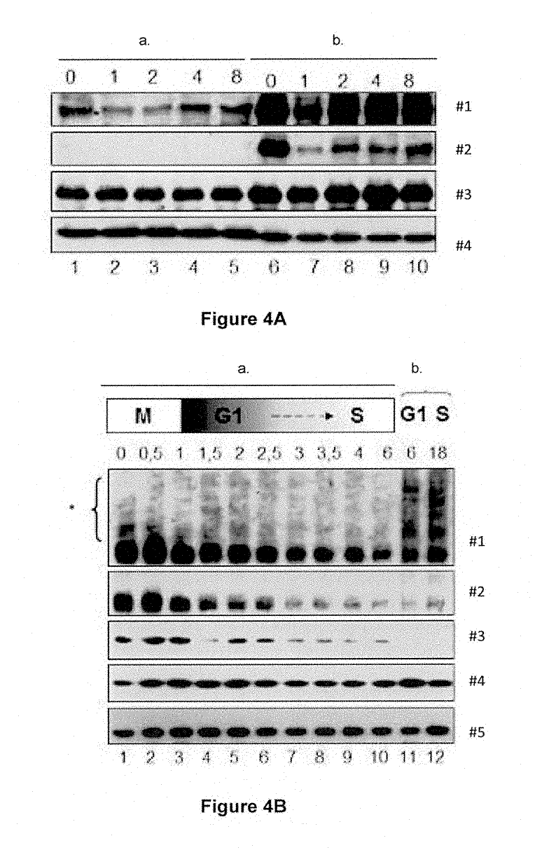

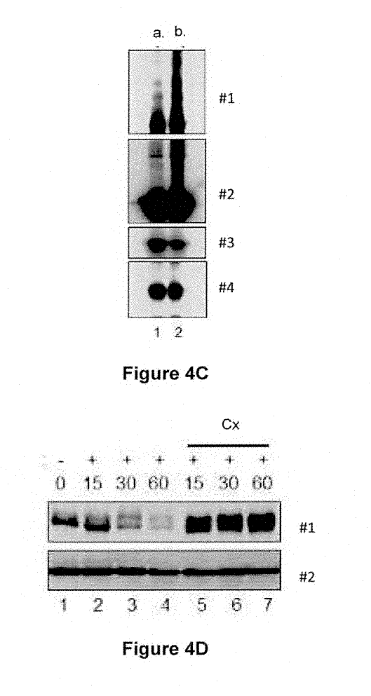

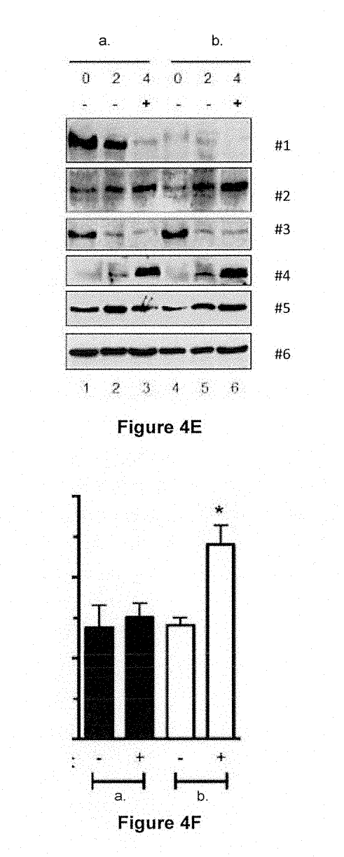

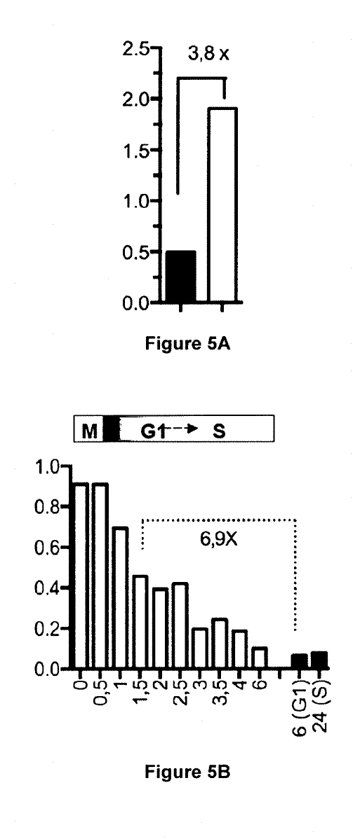

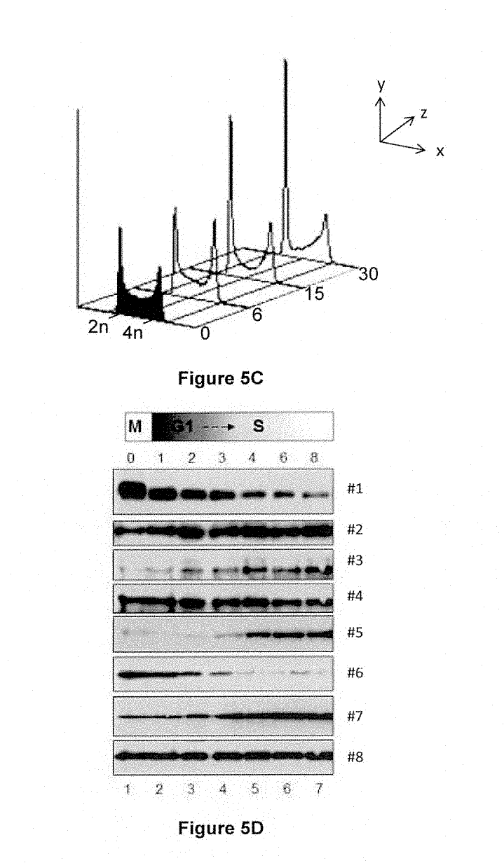

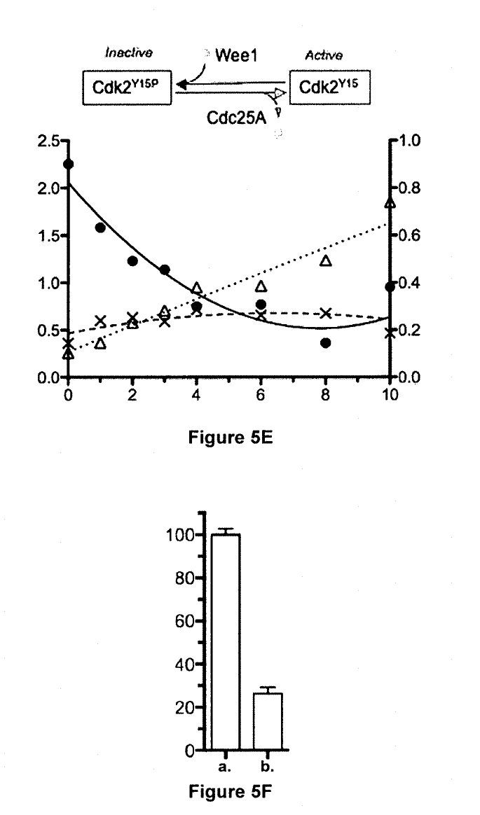

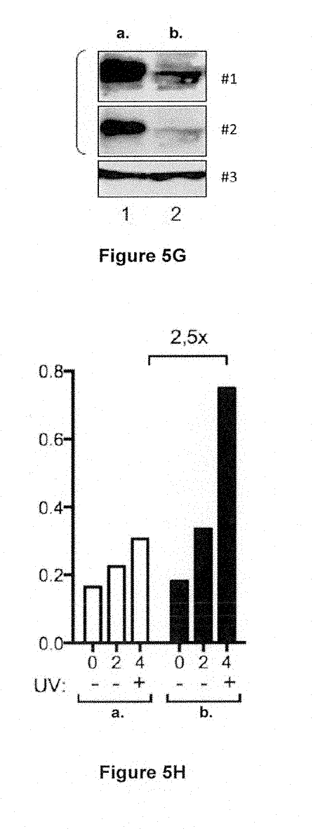

Persistent high levels of Cdc25A in ES cells sustain G1/S checkpoint bypass Cdc25A functions as a critical CDK2 regulator by removing an inhibitory phosphorylation on Tyrosine 15 (CDK2^{Y15P}) that in turn regulates S phase progression. The inventors compared Cdc25A and CDK2 protein abundance between ES cells and NIH-3t3 cells (FIG. 4A). Strikingly, while CDK2 abundance is marginally higher in ES cells, the levels of Cdc25A in asynchronously growing ES cells are exceedingly high compared to NIH-3t3 cells. As expected, upon UV-induced DNA damage, Cdc25A was degraded in both cell lines (FIG. 4A). However, one hour after irradiation, Cdc25A level remained about 4-fold higher in ES cells compared to unperturbed NIH-3t3 cells (lanes 1 and 7 and FIG. 5A), indicating that high levels of Cdc25A persist even upon UV-induced DNA damage. Since cell cycle distribution of asynchronously growing ES and NIH-3t3 cells is different, the inventors analysed Cdc25A abundance in synchronized cells (FIG. 4B). The inventors observed that in G1, ES cells contained about 7-fold more Cdc25A protein than NIH-3t3 cells (lanes 3 and 11 and FIG. 5B). Proteolysis of Cdc25A mediated by the E3 ubiquitin ligase APC^{Cdh1} occurs at mitotic exit. Polyubiquitylated forms appear as a polypeptide ladder of higher molecular weight than the unmodified protein. In NIH-3t3 cells synchronized in G1 and S phase, the inventors could observe such ladders by western blot using a specific Cdc25A antibody (FIG. 4B, dark). Strikingly, in synchronized ES cells, these isoforms are much less abundant, whereas levels of unmodified Cdc25A are 7-fold higher than in NIH-3t3 cells (FIG. 5B). Cdc25A immunoprecipitation from either ES or NIH-3t3 cells cotransfected with GFP-Cdc25A and HA-tagged ubiquitin, confirmed the presence of much more Cdc25A polyubiquitylated forms in NIH-3t3 than in ES cells (FIG. 4C).

Next the inventors tested whether incomplete Cdc25A degradation may be due to impaired function of the ATR-Chk1 pathway. To this end, the inventors treated cells with a Chk1 inhibitor and analyzed Cdc25A protein levels upon UV irradiation. In contrast to a previous report in which degradation of Cdc25A was not affected by both Chk1 and Chk2 inhibitors, the inventors observed that Cdc25A degradation in ES cells is entirely dependent on Chk1 activity (FIG. 4D).

Treatment of asynchronously growing ES cells with roscovitine (a selective CDKs inhibitor) induced dose-dependent increase of G1 cells and reduced the fraction of S phase cells (FIG. 5C), demonstrating that, similar to somatic cells, in ES cells CDK activity is necessary for the G1/S transition. Inhibitory CDK2^{Y15} phosphorylation is mediated by Wee1 kinase and relieved through dephosphorylation by Cdc25A. The inventors therefore analysed changes in protein level of Wee1, Cdc25A, and CDK2Y15P during G1/S transition in ES cells, which, according to BrdU uptake experiments, occurs between 2-3 hours after nocodazole release (FIG. 2C). Mitotic exit was monitored by histone H3 phosphorylation at serine 10 (H3^{S10P}), and S phase entry by H3 and Cyclin A production. Interestingly, Wee1 levels did not show significant cell cycle-dependent variations, while Cdc25A levels decreased and inversely correlated with CDK2^{Y15P} abundance (FIGS. 5D-E), suggesting that in ES cells, cell cycle-dependent fluctuation of Cdc25A levels may specifically regulate CDK2^{Y15P}.

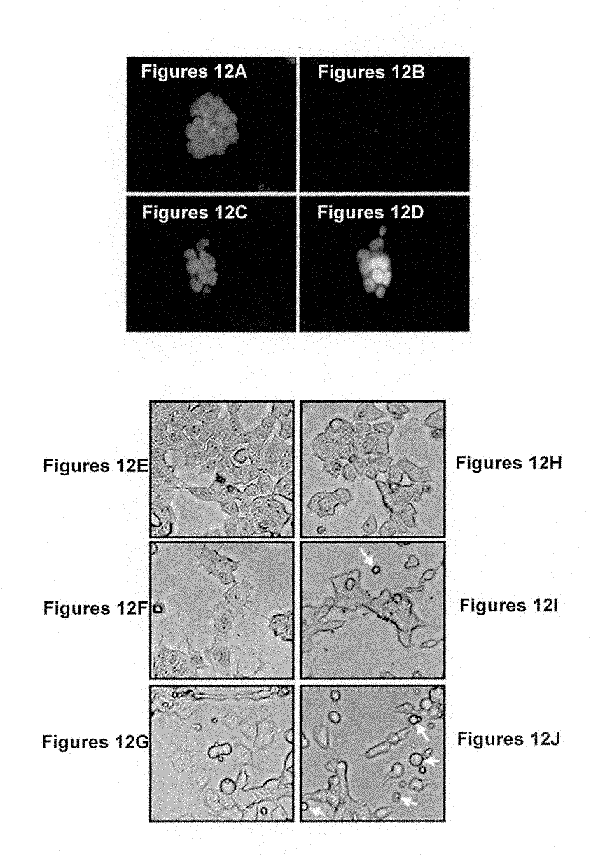

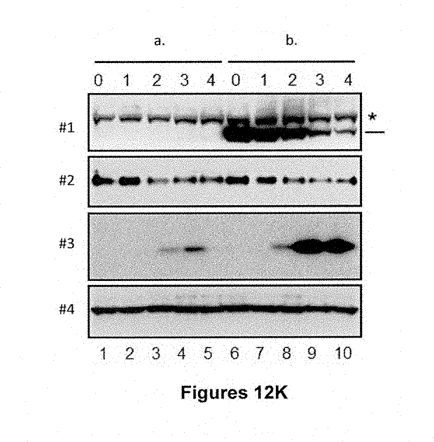

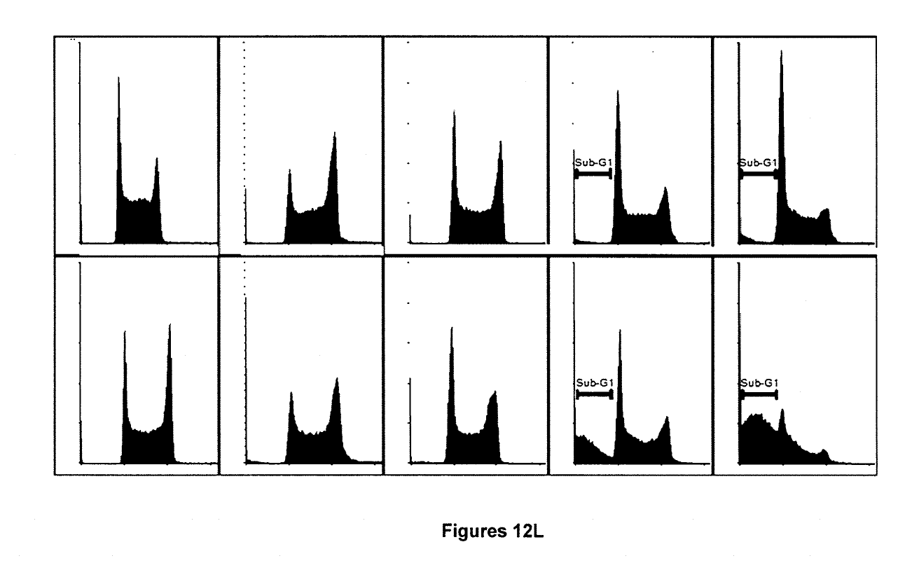

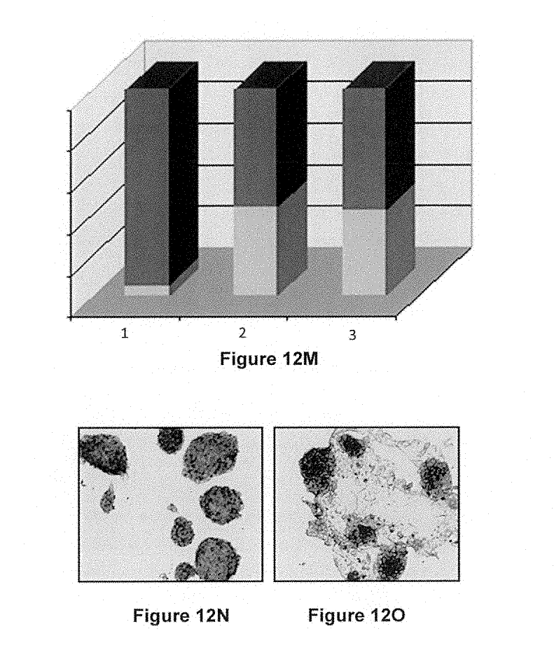

To further pinpoint the specific role of Cdc25A in the G1/S checkpoint, the inventors examined whether interfering with Cdc25A levels by RNAi affects S-phase entry upon DNA damage (FIGS. 5F-G). To avoid undesired differentiation of ES cells due to G1 phase extension upon Cdc25A downregulation that would interfere with the interpretation of this experiment (see below and FIGS. 12E-F), knockdown was performed over a short period (24 hours). Interestingly, Cdc25A knockdown (FIG. 4E) resulted in a significant, UV-dependent, increase of BrdU-negative cells with 2N DNA content (FIG. 4F) mirrored by increased CDK2^{Y15P} levels (FIG. 4E, compare lane 3 with lane 6 and FIG. 5H). Importantly, the slight increase of CDK^{2Y15P} levels between 2 and 4 hours after release (FIG. 4E, lane 3), also observed in synchronized undamaged cells entering S-phase (FIG. 5D), did not result in an apparent difference in S phase entry in mock and UV-treated cells transfected with control RNAi (FIG. 4F). Altogether, these data show that ES cells contain high levels of Cdc25A and that its knockdown leads to a UV-dependent G1 delay.

### ES Cells Express High Dub3 Deubiquitylase

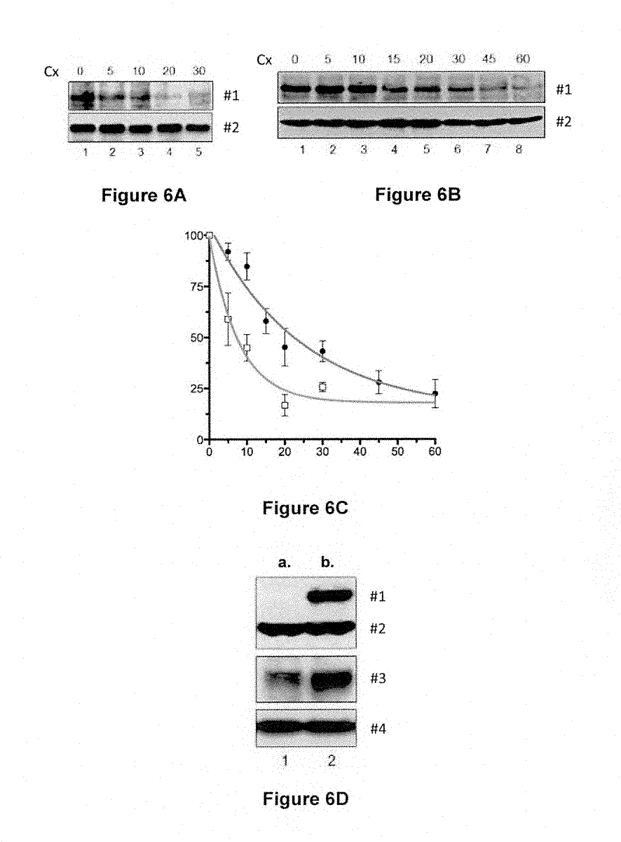

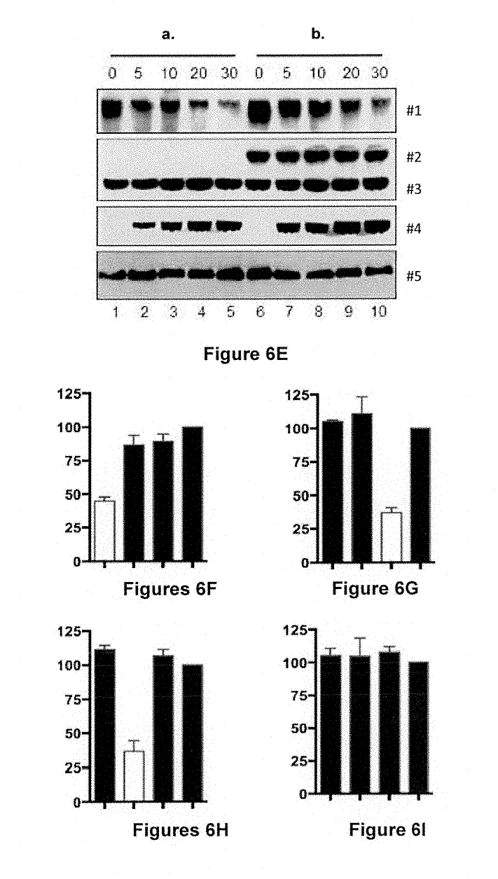

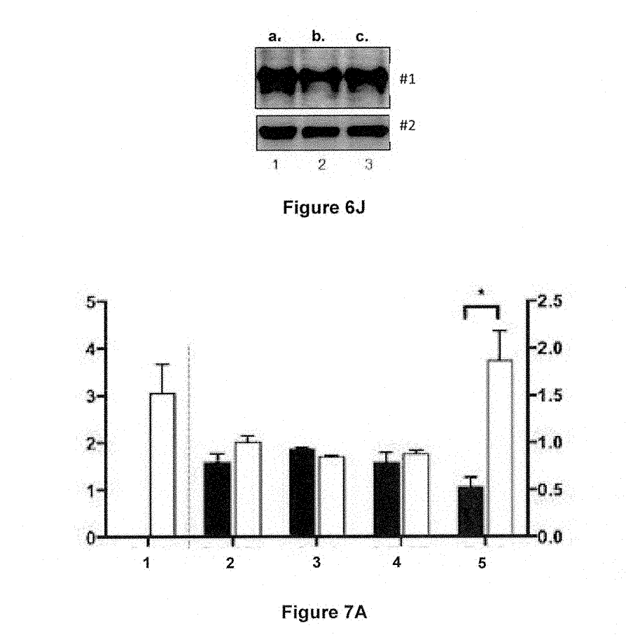

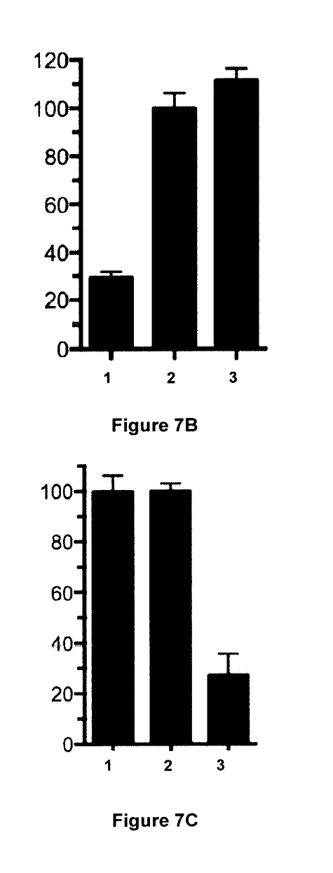

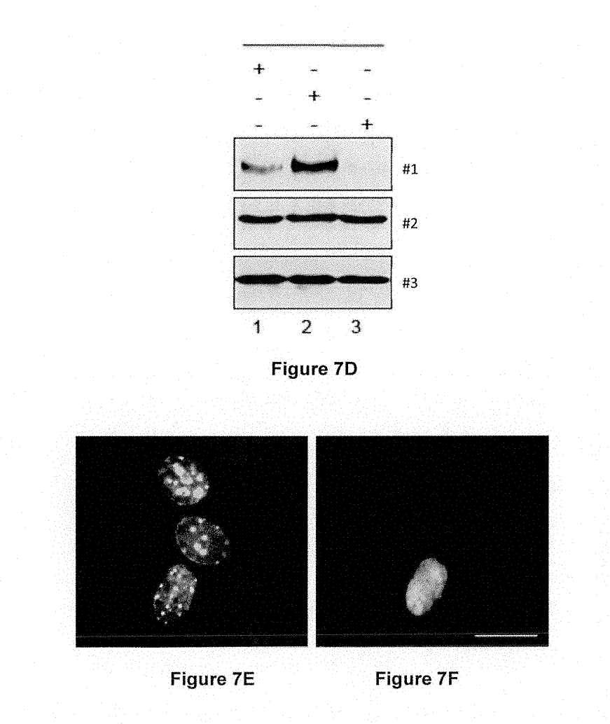

Elevated Cdc25A protein levels can be explained by increased gene expression, increased translation or reduced protein degradation. The inventors analysed protein turnover in the presence of cycloheximide to inhibit de novo protein synthesis (FIGS. 6A-C). Using this approach, the inventors found a 3-fold longer half-life of Cdc25A in ES cells (t_{1/2}=24 min) compared to NIH-3t3 cells (t_{1/2}=8 min). Of note, since unsynchronized cells were used, the inventors cannot exclude that the observed difference is partly due to distinct cell cycle distribution of both cell types. However, this data strongly suggests alterations in protein stability that, according to data shown in FIGS. 4B-C, might reflect differences between polyubiquitylation and ubiquitin removal by hydrolysation (deubiquitylation). To address this point, the inventors compared gene expression of Cdc25A, Cdh1, β-TrCP and that of the recently described Dub3 deubiquitylase, between ES and NIH-3t3 cells. Whereas mRNA levels of Cdc25A, Cdh1 and β-TrCP in ES cells hardly differ from NIH-3t3 cells, Dub3 mRNA level was 4-fold higher in ES cells (FIG. 7A). Moreover, RNAi-mediated knockdown of Dub3 in ES cells (FIG. 7B) did not affect Cdc25A mRNA level (FIG. 7C) but resulted in 3-fold reduction of Cdc25A protein abundance (FIG. 7D). These data are consistent with previous work in human cells and indicate that Dub3 function in regulating Cdc25A protein stability is analogous in mouse ES cells. In addition, the inventors also observed a role of Dub3 in Cdc25A stability in unperturbed and damaged NIH-3t3 cells (FIG. 6D-E). Of note, GFP-tagged Dub3 shows an exclusive nuclear localization (FIG. 7E-F) as previously observed for Cdc25A in ES cells. Finally, to address the role of Cdh1 and β-TrCP in regulating Cdc25A levels in ES cells, the inventors performed RNAi-mediated knockdown experiments. In contrast to Dub3 knockdown neither Cdh1, nor β-TrCP downregulation affected Cdc25A mRNA expression nor did significantly alter Cdc25A stability (FIGS. 6F-J). These observations are consistent with a previous study showing that APC/Cdh1 activity is attenuated in ES cells by high levels of the Emi1 inhibitor.

### Orphan Receptor Esrrb Regulates Dub3 Gene Expression

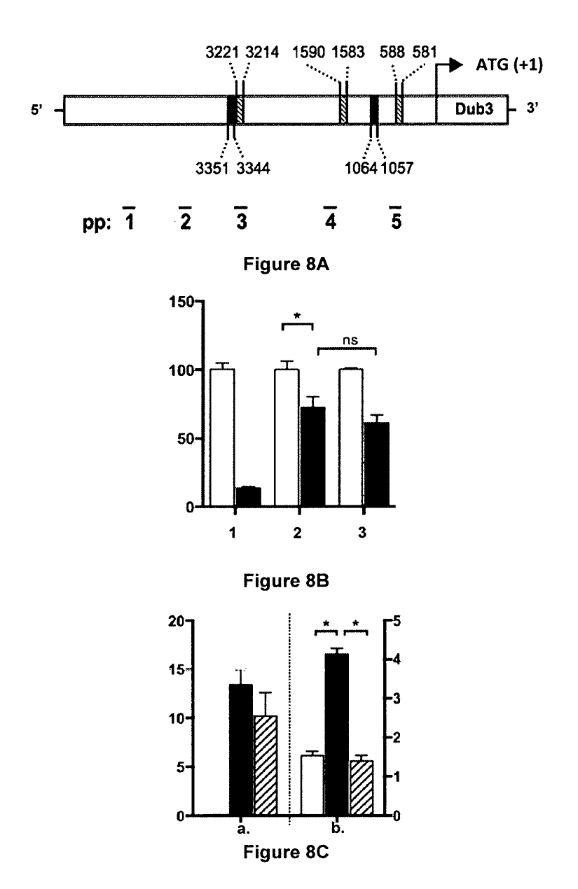

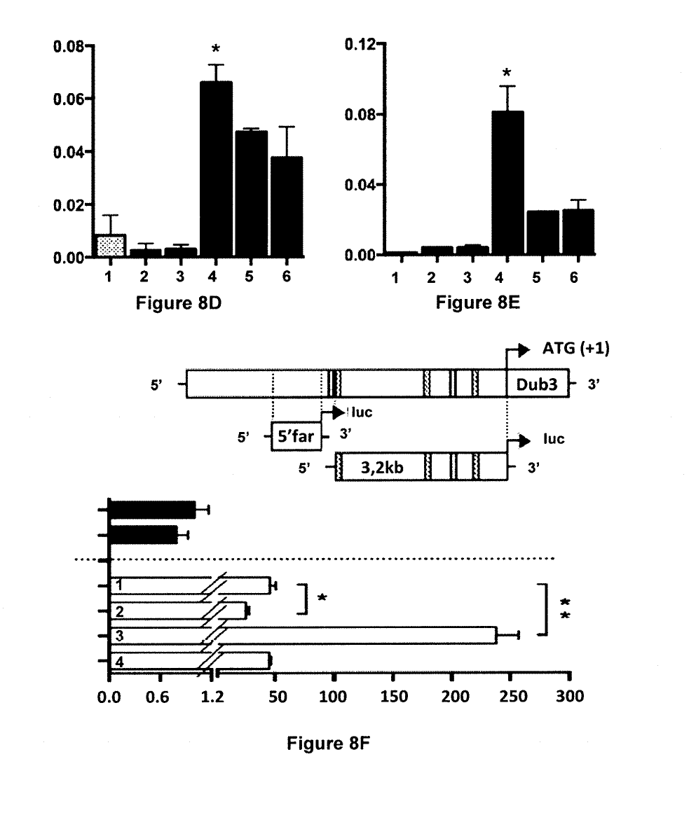

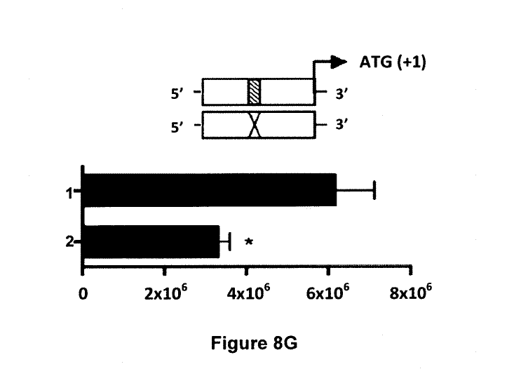

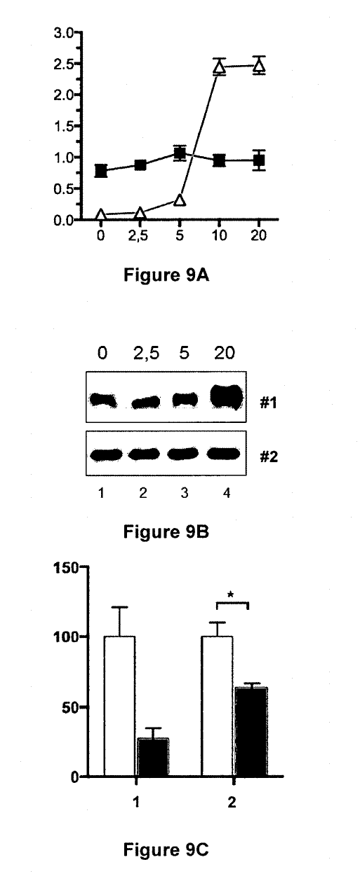

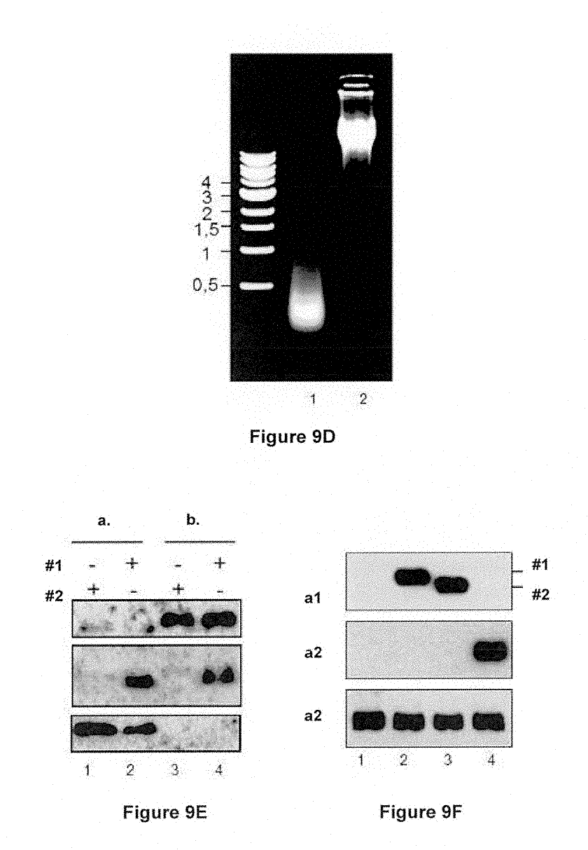

Based on previously described consensus sequence for binding motifs of key transcription factors involved in reprogramming, the inventors analyzed the proximal promoter (6 kb) of the Dub3 gene. Strikingly, while no Oct4, Nanog, Klf4, Smad1, Stat3, c-Myc nor n-Myc consensus sites could be detected, the inventors originally (NCBI37/mm9) found up to seven estrogen-related-receptor-b (Esrrb) putative binding motifs (consensus: 5′-TNAAGGTCA-3′) and two Sox2 putative response elements (consensus: 5′-CATTGTT-3′). However the latest update of this genomic sequence (GRCm38/mm10) displays only three Esrrb sites (FIG. 8A, Esrrb-RE). Esrrb is a nuclear receptor belonging to the superfamily of nuclear hormone receptors. Together with Sox2, it is part of the core self-renewal machinery. Esrrb knockdown using a previously validated RNAi sequence resulted in significant decrease of endogenous Dub3 transcript level (FIG. 8B), to a similar extent than the previously described Esrrb target gene Nanog. Inversely, ectopic expression of Esrrb in ES cells, and not of its C-terminal truncated form (Δ-Cter) lacking the activation function 2 (AF2) domain, led to significant increase in endogenous Dub3 mRNA level (FIG. 8C). Moreover, treatment of ES cells with increasing dose of DY131, a previously described selective Esrrb and Esrrg agonist, boosted Dub3 gene expression and increased Cdc25A protein abundance without affecting Cdc25A transcript level (FIGS. 9A-B). Inversely, Esrrb knockdown resulted in a 40% decrease of DY131-mediated Dub3 transcription (FIG. 9C), while Sox2 knockdown using a previously published RNAi sequence did not strongly affected Dub3 expression, though slightly increased it (inventors unpublished observations).

Next, the inventors performed chromatin immunoprecipitation (ChIP) experiments to map Esrrb and Sox2 binding to Dub3 promoter in ES cells. To this end, the inventors designed five primer pairs (FIG. 8A, pp) separated by approximately 1 kb to scan promoter occupancy by Esrrb and Sox2 within the 6 kb upstream of the start codon (ATG+1). Sonication of chromatin resulted in fragments under 500 bp, limiting signal overlap between primers (FIG. 9D). ChIP analysis with an anti-Esrrb antibody (FIG. 9E) shows that Esrrb binds to the proximal Dub3 promoter in regions containing the three Esrrb consensus binding motifs (FIG. 8D, pp 3-5), while no Esrrb binding was observed in an upstream region that does not contain Esrrb binding sites (pp 1-2). On the contrary, ChIP analysis with an anti-Sox2 antibody showed high enrichment only at one of the two consensus sites in the Dub3 promoter (Sox2-RE2), around primer pair 3, while in the region containing the second site (Sox2-RE1, pp4-5) Sox2 was bound to much lower levels.

To corroborate abovementioned ChIP data, the inventors cloned the Dub3 proximal promoter (3.2 kb) and analyzed its transcriptional activity in a reporter assay using luciferase activity as readout. For this purpose the inventors used cells that have very low expression of endogenous steroid receptors (CV1 cells). As anticipated, the inventors observed strong induction of luciferase activity upon Esrrb expression in cells cotransfected with the 3.2 kb Dub3 promoter that contains all three Esrrb binding sites (FIG. 8E, Esrrb, white bars) while only background activity was observed on a region of the Dub3 promoter (5′ far) devoid of Esrrb consensus binding sites (Esrrb, black bars). Similarly, expression of Esrrb Δ-Cter, resulted in basal promoter activity, comparable to that observed by expression of empty vector (EV, FIG. 8E and FIG. 9F). Interestingly, the inventors did not observe stimulation of luciferase activity upon expression of Sox2, but a small and significant repression of basal promoter activity (FIG. 8E). Importantly, mutation of the unique Esrrb binding site in a 1 kb Dub3 genomic fragment decreased transcriptional activity (FIG. 8F). Altogether these observations suggest that Dub3 is a direct Esrrb target gene, having a positive role in regulating transcription of the Dub3 gene, while Sox2 on its own is not sufficient to stimulate Dub3 transcription.

### Developmental Regulation of Dub3 Expression and Cdc25A Stability

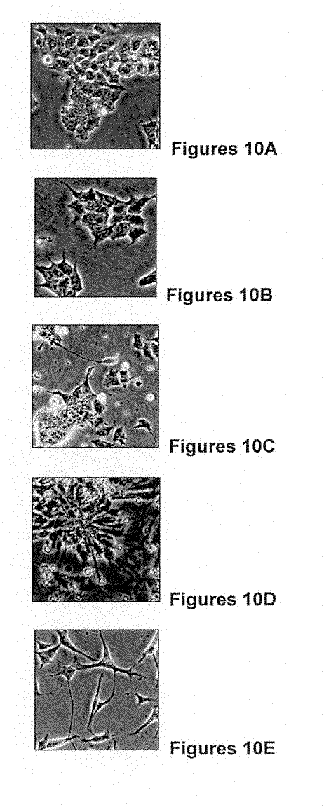

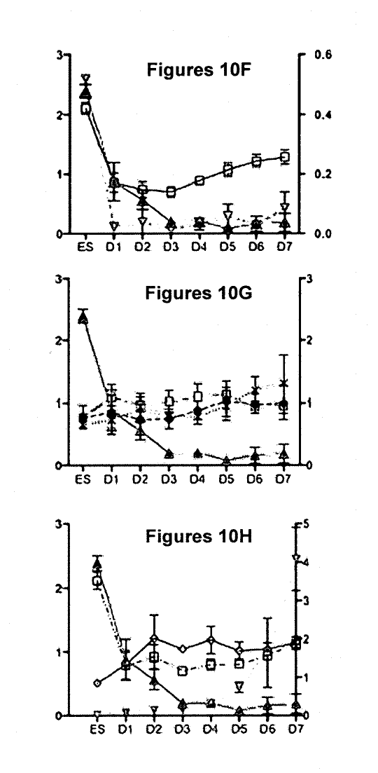

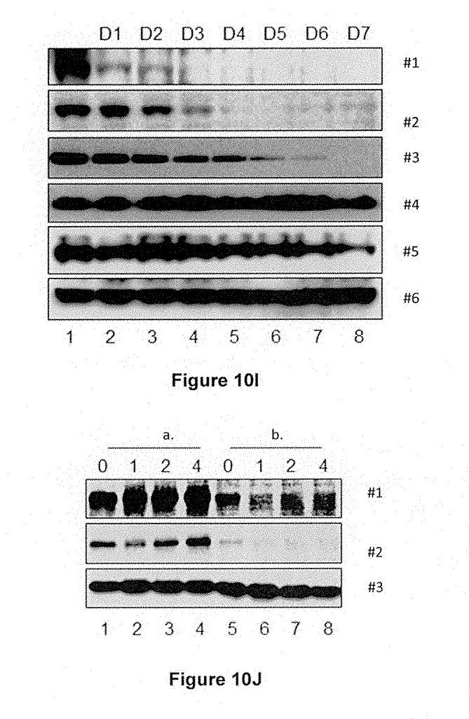

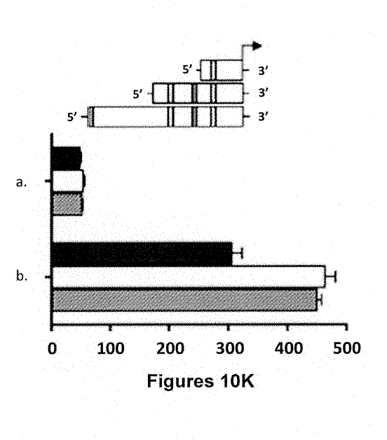

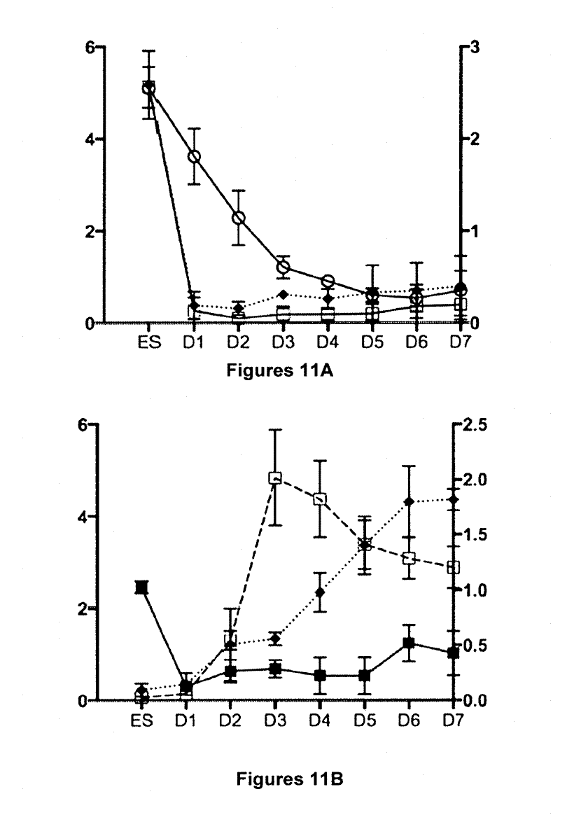

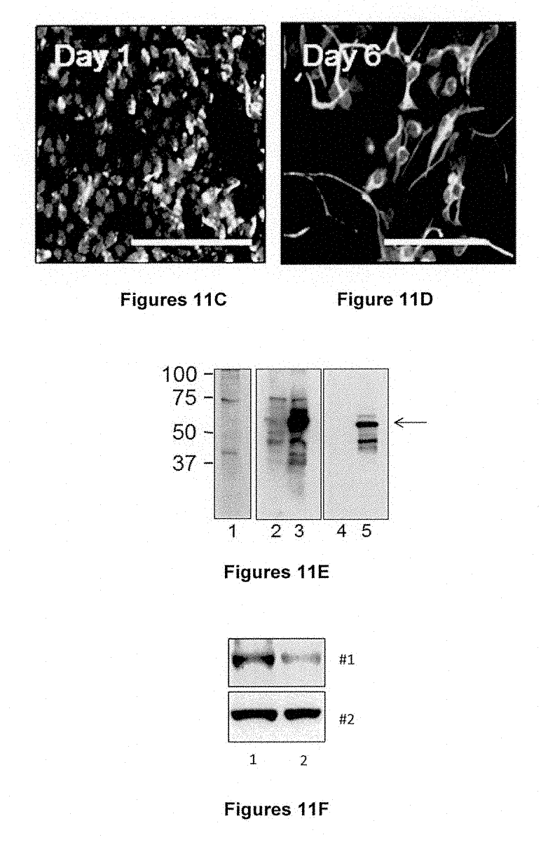

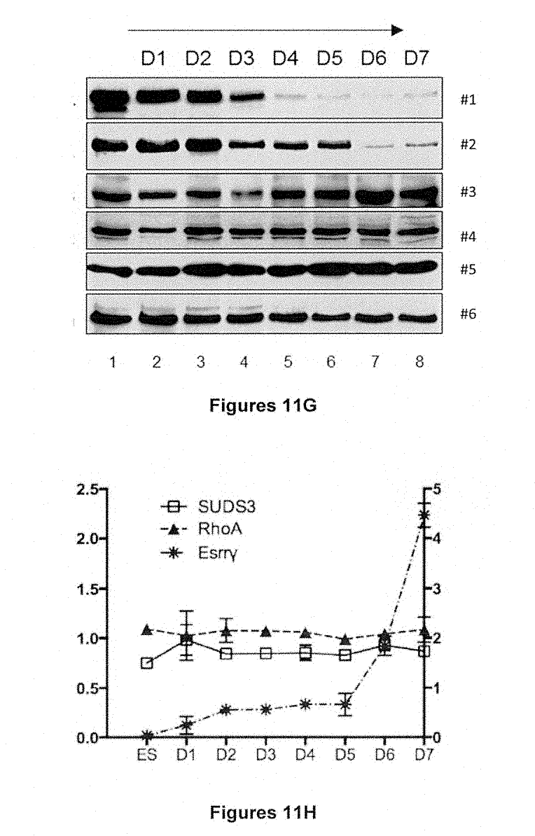

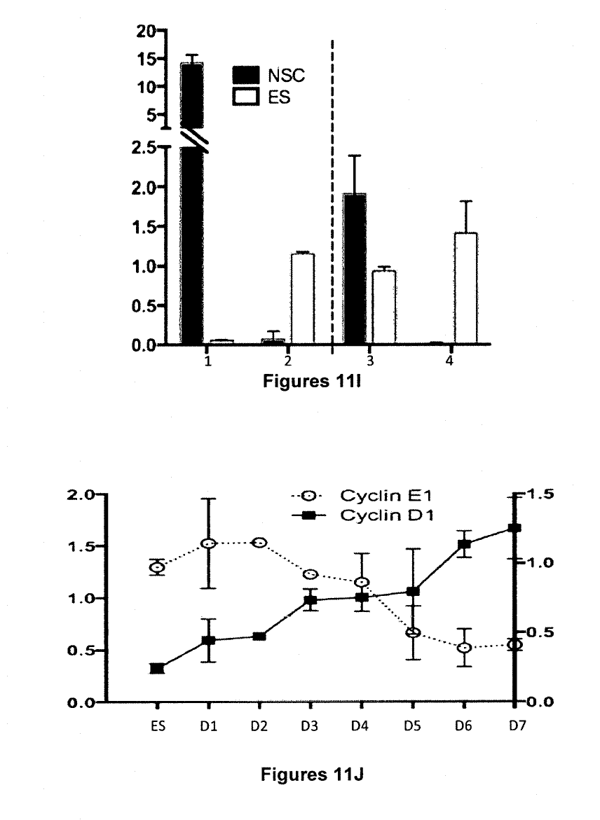

Esrrb is a pluripotency factor highly expressed in ES cells that, unlike Sox2, is strongly downregulated upon ES differentiation. Since Dub3 is an Esrrb target, the inventors analyzed expression of Dub3 during neural conversion of ES cells in vitro. Plating of ES cells in N2B27 culture medium triggers conversion into neuroepithelial precursors microscopically visible as rosette conformations (FIGS. 10A-E, day 7, in particular FIG. 10E). Loss of pluripotency was monitored by expression analysis of specific markers such as Oct4, Nanog, Klf4, and acquisition of neural identity was monitored by Nestin and Sox1 expression. Specificity was controlled by analysis of Sox7 expression, a well-established endoderm marker (FIGS. 11A-D). Importantly, Nestin was detectable in just about each individual cell of the differentiating population at day 6, indicating homogenous neural conversion. Acute (within 24 hours) decrease of Esrrb mRNA expression preceded in time a marked and dramatic decrease of Dub3 expression (FIGS. 10F-H). Expression of Sox2 also decreased after 24 hours, however of only 50% and increased afterwards. In contrast, neither Cdc25A nor Cdh1 or β-TrCP transcript levels significantly changed during differentiation (FIG. 10G). Expression analysis of three other deubiquitylases implicated in Cdc25A stability, USP13, 29 and 48 revealed a decrease of only USP48 within 24 hours after differentiation (FIG. 10H) that mirrored Sox2 expression. Importantly, the inventors could not find any consensus Esrrb binding sites within the USP48 proximal promoter. In contrast, USP13 gene expression did not significantly change during differentiation, while USP29 expression strongly increased during neural conversion.

To analyze Dub3 protein levels the inventors raised a specific antibody recognizing, as expected, a 60 kDa polypeptide in SDS-PAGE (FIGS. 11E-F). Dub3 protein levels dropped massively very early during differentiation, much earlier than Oct4, finely correlating with Dub3 mRNA levels (FIG. 10I). Strikingly, lineage commitment between days 2-3, as monitored by Sox1 expression, led to a marked and continuous decrease of Cdc25A protein level, while the protein level of the two other Cdc25 family members, Cdc25B and Cdc25C, remained constant during differentiation (FIG. 11G). The inventors further analyzed expression of two additional Dub3 substrates during differentiation, RhoA and Suds3, and observed no significant variations in gene expression (FIG. 11H), nor in protein stability (FIG. 10I), although a small decrease in Suds3 level was seen at day 7 after differentiation. Finally, the inventors found very low expression of Esrrg (another member of the subfamily) in ES cells that further increased during differentiation (FIG. 11H), corroborating the specificity of Dub3 gene regulation by Esrrb. Altogether, these findings suggest that reduced Cdc25A protein abundance during neural differentiation is likely governed at the post-translational level.

While retaining self-renewal properties, neural stem cells (NSC) are multipotent stem cells derived from ES cells, isolated and amplified at day 7 following differentiation. Quantification of Cdc25A abundance revealed 8-fold more Cdc25A in asynchronously growing ES cells compared to NSCs (FIG. 10J). Similar to NIH-3t3 cells, the inventors detected very low Dub3 transcript levels in NSCs (FIG. 11I). Finally, the inventors isolated and analyzed three different genomic fragments of the Dub3 promoter and compared basal transcriptional activity in NIH-3t3 versus ES cells. The inventors observed strong transcriptional activity of all three promoter sequences in ES cells, about 10-fold higher than in NIH-3t3 cells (FIG. 10K), further corroborating mRNA expression during differentiation (FIGS. 10F-H).

### Dub3 Expression is Important for Maintenance of Pluripotency and Cell Cycle Remodelling During Differentiation

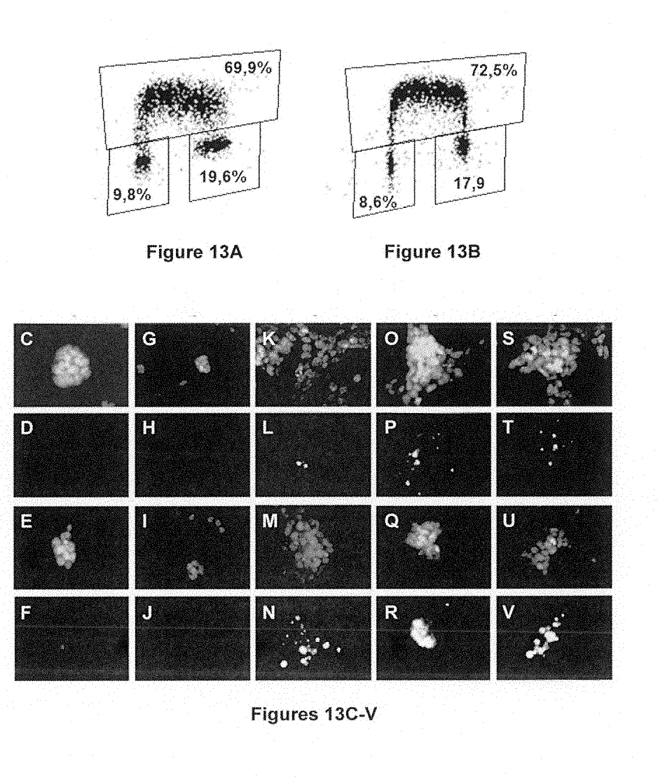

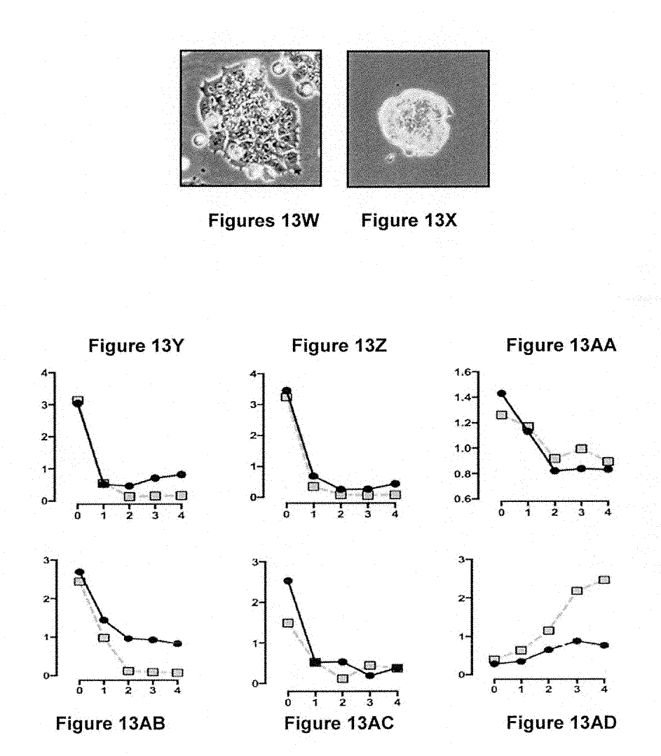

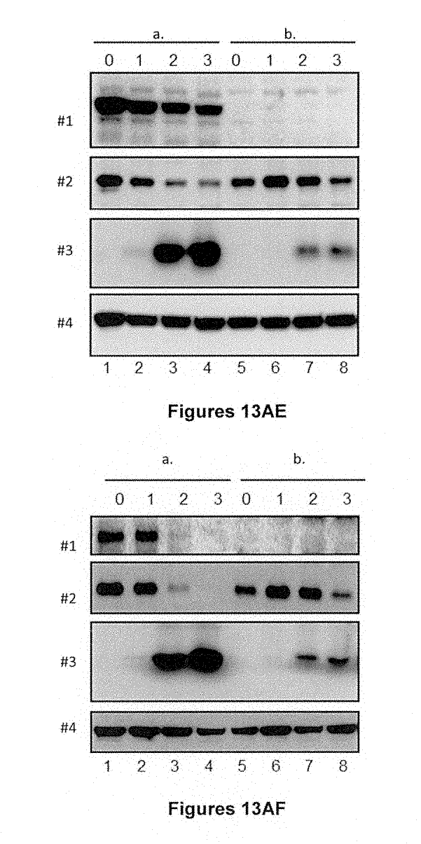

Stable transfection of Esrrb in ES cells has been shown to be sufficient to sustain pluripotency in absence of LIF. The inventors therefore addressed whether forced Dub3 expression in ES cells could substitute Essrb function in maintaining pluripotency in absence of LIF. To this end, the inventors generated a stable ES cell line, expanded from a single ES colony, expressing eGFP-Dub3 under control of a constitutive strong promoter (FIGS. 12A-D). Remarkably, while authors reported that high Dub3 expression induces S-G2/M arrest in human somatic U2OS cells, ES cells overexpressing Dub3 could be propagated without significant differences in cell cycle distribution compared to a control cell line, indicating that in ES cells constitutive Dub3 expression is not toxic (FIGS. 13A-B and W-X). Removal of LIF led to an apparent highly similar morphological differentiation program in both cell-lines, but unexpectedly resulted in massive death of eGFP-Dub3-expressing ES cells two days after, microscopically visible as detached cells with retracted nuclei (FIGS. 12E-J, arrows). Of note, five days following LIF withdrawal, hardly any cell survived in the eGFP-Dub3 expressing cell-line. Caspase-3 activity, essential for proper differentiation, was higher at days 3-4 in eGFP-Dub3 expressing cells compared to empty vector, strongly indicative of apoptosis (FIG. 12K and FIG. 13C-V). Finally, whereas mRNA and protein levels of pluripotency and differentiation markers were highly comparable in both cell lines, the inventors observed elevated expression of the apoptotic marker Noxa at day two and afterwards in eGFP-Dub3 expressing cells (FIG. 13Y-AD).

Remarkably, 2-3 days upon LIF removal, a strong reduction of eGFP-Dub3 protein level was evident (FIG. 12K), suggesting an additional control at post-transcriptional level, very likely proteolysis, occurring during differentiation. A similar phenotype was observed upon N2B27-mediated neural conversion, and a similar result was also observed with a ES cell line expressing HA N-terminal-tagged Dub3 (FIGS. 13AE-AF), ruling out a non-specific effect of the GFP tag or of the differentiation protocol used. Onset of apoptosis, was equally observed by FACS analysis (FIG. 12L), that showed the presence of subdiploid (less than 2N) cell debris starting from day three during differentiation and being predominant at day four. Interestingly, appearance of the sub-G1 cell population in ES cells expressing eGFP-Dub3 was concomitant to cell lineage commitment, as monitored by Sox1 and Nestin expression (FIGS. 11A-B) and cell cycle remodelling which started at day three in the control cell line (empty vector), resulting in lengthening of the G1 phase (FIG. 12L). Altogether these results strongly suggest that high Dub3 expression is lethal during differentiation at the time when cell cycle remodelling occurs.

Finally the inventors analyzed the effect of Dub3 or Cdc25A knockdown in ES cells. Interestingly, prolonged (7 days) RNAi mediated Dub3 knockdown, resulted in an increase of alkaline phosphatase (AP)-negative colonies, as well as heterogeneous morphological differentiation of ES cells even in the presence of LIF, suggesting that Dub3 expression is important for maintenance of pluripotency (FIGS. 12M-O). A very similar result was also observed upon prolonged Cdc25A knockdown. In sum, these data couple the self-renewal machinery of ES cells through Essrb to the master cell cycle regulator Cdc25A and remodelling of the cell cycle during differentiation through modulation of Dub3 expression.

### Discussion

In this study the inventors dissected the G1/S checkpoint signalling pathway in ES cells. The inventors found that ES cells maintain high levels of the Cdc25A phosphatase in Cl that persists even after DNA damage. Knockdown of Cdc25A expression resulted in a G1 delay and increased CDK2Y15P after UV damage within 24 hours post RNAi treatment (a condition required to avoid natural G1 phase expansion due to differentiation of ES cells). Indeed, prolonged Cdc25A downregulation (or Dub3), resulted in cell differentiation in the presence of LIF, in line with the notion that lengthening of the G1 phase and deregulation of CDK2 activity is linked to differentiation. These findings provide an explanation for absent regulation of CDK2 activity upon DNA damage in ES cells. This model is also in line with existing evidence linking elevated Cdc25A expression with impaired G1/S arrest followed by radioresistant DNA synthesis in cancer cells.

Interestingly, in addition to Cdc25A, the inventors have also observed down-regulation of Cyclin E (FIG. 11J), another CDK2 regulator that is rate limiting during the G1/S transition and opposes spontaneous differentiation of naïve ES cells. Moreover, ablation of the SCFFbw7-mediated degradation pathway controlling Cyclin E abundance in vivo results in impaired differentiation, genomic instability and hyperproliferation, illustrating the importance of Cyclin E regulation in mouse development. Taken together, both reduced abundance of Cdc25A and Cyclin E during differentiation of ES cells, likely embody key molecular adaptations that control CDK activity and consequent G1 lengthening. Importantly, as a result of expanded G1, the p53-dependent response may now become more effective in CDK2 inhibition since this requires a slow transcriptional-dependent induction of the CDK inhibitor p21 protein level. It is anticipated that p21 may have virtually no role in CDK2 regulation in ES cells since these cells spend most of their time in S phase and p21 is efficiently degraded by the PCNA-dependent CRL4Cdt2 ubiquitin ligase throughout S phase, as well as after DNA damage.

The inventors have provided evidence that post-transcriptional regulation of Cdc25A abundance in ES cells depends upon the Dub3 deubiquitylase. Expression of Dub3, and not Cdh1 or β-TrCP, is higher in ES cells compared to differentiated cells, and knockdown of Cdh1 or β-TrCP did not significantly change the stability of Cdc25A since it is already highly stabilized in ES cells. These observations are consistent with the finding that ES cells have attenuated APC activity that increases during differentiation. Of the four additional deubiquitylases implicated in Cdc25A stability in human cells (USP13, 29, 48 and Dub2A), the inventors found that only USP48 mRNA levels significantly decreased during differentiation although its expression remained high and increased towards the end of differentiation, mirroring Sox2 expression. Hence, although the inventors cannot exclude a redundant role for Dub2A and USP48 in Cdc25A stability during differentiation, the inventors data support a key role for Dub3 in this process, as previously shown in somatic cells, and suggest that in ES cells the balance of ubiquitylation and deubiquitylation activities, which fine-tunes the steady-state level of Cdc25A, is shifted towards deubiquitylation due to high Dub3 expression.

The inventors showed that downregulation of Esrrb negatively affected the endogenous expression of the Dub3 gene, to a similar extent than a previously characterized Esrrb target gene, Nanog. However, expression of Oct4, another Esrrb target was not found to be much affected by Esrrb knockdown. These differences likely exist because in ES cells, expression of pluripotency genes is under the combinatorial control of transcription factors of the pluripotency gene regulatory network. This transcriptional control appears to be very complex, gene-specific and remains to be further clarified.

The inventors observed that while forced Dub3 expression could not inhibit differentiation upon LIF withdrawal, unexpectedly it induced massive apoptosis during differentiation concomitant to lineage commitment and cell cycle remodelling, such as lengthening of the G1 phase. These observations are in line with the recent finding that expression of non-degradable Cdc25A mutants leads to early embryonic lethality in mice (E3.5) showing the importance of fine-tuning the expression level of Cdc25A already at the oocyte and morula stages. Although the inventors have shown that Cdc25A is a critical Dub3 substrate in ES cells, the inventors cannot exclude the implication of other Dub3 substrates in the toxicity observed by forced Dub3 expression during differentiation. The importance of tight Cdc25A regulation during embryogenesis is also underscored by its function in regulation of pluripotency versus differentiation of ES cells since Cdc25A is expressed in progenitor cells undergoing proliferative self-renewing divisions. The inventors speculate that this developmental regulation might be governed by Dub3 to modify cell cycle dynamics under control of Esrrb.

In conclusion the inventors' results couple the Cdc25A-CDK2 cell cycle signalling pathway to the self-renewal machinery through Esrrb-dependent regulation of Dub3 in ES cells, and highlight the importance of deubiquitylases in stem cell and developmental biology. Since cell cycle regulation is a rate-limiting step in reprogramming processes, these findings put Dub3 and Cdc25A as interesting candidate genes in cell reprogramming.

1. A method for modulating cell differentiation comprising the administration to a determined cell:
Dub3 protein, said protein comprising the amino acid sequence as set forth in SEQ ID NO: 1, or any variant thereof having at least 43% identity with said amino acid sequence SEQ ID NO: 1, and having ubiquitin hydrolase activity or
a nucleic acid molecule coding for said protein or said variant thereof, or
an inhibitor of the activity, i.e. the ubiquitin hydrolase activity and/or of the expression of said protein or said variant thereof.

2. The method according to claim 1, for modulating totipotent or pluripotent cell differentiation.

3. A method for inducing dedifferentiation of differentiated cells, the cells obtained from the dedifferentiation of differentiated cells being iPS cells, the method comprising a step of administering to a differentiated cells
Dub3 protein, said protein comprising the amino acid sequence as set forth in SEQ ID NO: 1, or any variant thereof having at least 43% identity with said amino acid sequence SEQ ID NO: 1, or
a nucleic acid molecule coding for said protein or said variant thereof.

4. The method according to claim 3 for inducing dedifferentiation of differentiated cells, wherein said cells Dub3 protein or said nucleic acid molecule coding for said protein are associated with at least an Oct family member protein and a Sox family member protein.

5. The method according to claim 3, wherein said Dub3 protein is expressed in said iPS cells at a level corresponding to at least 2 fold lower than the expression of said Dub3 protein in totipotent cell.

6. A method for inducing a spontaneous differentiation of totipotent or pluripotent cells, comprising the administration to a determined cell of an inhibitor of the activity and/or of the expression of the Dub3 protein or a variant thereof, said protein comprising the amino acid sequence as set forth in SEQ ID NO: 1, or any variant thereof having at least 43% identity with said amino acid sequence SEQ ID NO: 1, and having ubiquitin hydrolase activity.

7. A method for the treatment of therapy-resistant tumors comprising a step of administering to a patient in a need thereof of one of:
the Dub3 protein, said protein comprising the amino acid sequence as set forth in SEQ ID NO: 1, or any variant thereof having at least 43% identity with said amino acid sequence SEQ ID NO: 1, or
a nucleic acid molecule coding for said protein or said variant thereof, or
an inhibitor of the activity and/or of the expression of said protein or said variant thereof.

8. The method according to claim 7, comprising a step of administering to a patient in a need thereof of an inhibitor of the activity of the Dub3 protein, i.e. the ubiquitin hydrolase activity and/or of the expression of said protein, said inhibitor being chosen among siRNA, miRNA, shRNA, RNA antisense, DNA antisense, antibodies or chemical compounds.

9. The method according to claim 8, wherein said inhibitor is a siRNA comprising the following amino acid sequence: SEQ ID NO: 41 or SEQ ID NO: 42.

10. A method for inducing cell death of differentiating cells, comprising a step of contacting differentiating cells with one of
the Dub3 protein, said protein comprising the amino acid sequence as set forth in SEQ ID NO: 1, or any variant thereof having at least 43% identity with said amino acid sequence SEQ ID NO: 1 and having ubiquitin hydrolase activity, or
a nucleic acid molecule coding for said protein or said variant thereof.


SEQUENCE LISTING (43 sequences)

SEQ ID NO: 1 - protein, 530 aa, Homo sapiens
MEDDSLYLGGEWQFNHFSKLTSSRPDAAFAEIQRTSLPEKSPLSCETRVDLCDDLAPVARQLAPREKLPLSSRRPAAVGAGLQNMGNTCYVNASLQCLTYTPPLANYMLSREHSQTCHRHKGCMLCTMQAHITRALHNPGHVIQPSQALAAGFHRGKQEDAHEFLMFTVDAMKKACLPGHKQVDHHSKDTTLIHQIFGGYWRSQIKCLHCHGISDTFDPYLDIALDIQAAQSVQQALEQLVKPEELNGENAYHCGVCLQRAPASKTLTLHTSAKVLILVLKRFSDVTGNKIAKNVQYPECLDMQPYMSQPNTGPLVYVLYAVLVHAGWSCHNGHYFSYVKAQEGQWYKMDDAEVTASSIISVLSQQAYVLFYIQKSEWERHSESVSRGREPSALGAEDTDRRATQGELKRDHPCLQAPELDEHLVERATQESTLDHWKFLQEQNKTKPEFNVRKVEGTLPPDVLVIHQSKYKCGMKNHHPEQQSSLLNLSSTTPTHQESMNTGTLASLRGRARRSKGKNKHSKRALLVCQ

SEQ ID NO: 2 - protein, 540 aa, Mus musculus
MVVSLSFPEETGGENLPSAPLEDSSKFFEEVFGDMVVALSFPEADPALSSPDAPELHQDEAQVVEELTTNGKHSLSWESPQGPGCGLQNTGNSCYLNAALQCLTHTPPLADYMLSQEHSQTCCSPEGCKMCAMEAHVTQSLLHSHSGDVMKPSQILTSAFHKHQQEDAHEFLMFTLETMHESCLQVHRQSDPTPQDTSPIHDIFGGWWRSQIKCLHCQGTSHTFDPFLDVPLDISSAQSVNQALWDTGKSEELLGENAYYCGRCRQKMPASKTLHVHIAPKVLLLVLKRFSAFTGNKLDRKVSYPEFLDLKPYLSEPTGGPLPYALYAVLVHDGATSNSGHYFCCVKAGHGKWYKMDDTKVTRCDVTSVLNENAYVLFYVQQTDLKQVSIDMPEGRVHEVLDPKYQLKKSRRKKRKKQCHCTDDAGEACENREKRAKKETSLGEGKVPQEVNHEKAGQKHGNTKLVPQEQNHQRAGQNLRNTEVELDLPVDAIVIHQPRSTANWGTDAPDKENQPWHNGDRLLTSQGLMSPGQLCSQGGR

SEQ ID NO: 3 - protein, 530 aa, Homo sapiens [sequence omitted]

SEQ ID NO: 4 - protein, 530 aa, Homo sapiens
MGDDSLYLGGEWQFNHFSKLTSSRPDAAFAEIQRTSLPEKSPLSSETRVDLCDDLAPVARQLAPREKLPLSSRRPAAVGAGLQNMGNTCYENASLQCLTYTLPLANYMLSREHSQTCQRPKCCMLCTMQAHITWALHSPGHVIQPSQALASGFHRGKQEDVHEFLMFTVDAMKKACLPGHKQVDHHSKDTTLIHQIFGGCWRSQIKCLHCHGISDTFDPYLDIALDIQAAQSVKQALEQLVKPEELNGENAYHCGLCLQRAPASNTLTLHTSAKVLILVLKRFSDVAGNKLAKNVQYPECLDMQPYMSQQNTGPLVYVLYAVLVHAGWSCHDGHYFSYVKAQEGQWYKMDDAEVTVCSITSVLSQQAYVLFYIQKSEWERHSESVSRGREPRALGAEDTDRRAKQGELKRDHPCLQAPELDEHLVERATQESTLDHWKFLQEQNKTKPEFNVGKVEGTLPPNALVIHQSKYKCGMKNHHPEQQSSLLNLSSTTRTDQESMNTGTLASLQGRTRRAKGKNKHSKRALLVCQ

SEQ ID NO: 5 - protein, 530 aa, Homo sapiens
MEDDSLYLGGDWQFNHFSKLTSSRLDAAFAEIQRTSLSEKSPLSSETRFDLCDDLAPVARQLAPREKLPLSSRRPAAVGAGLQKIGNTFYVNVSLQCLTYTLPLSNYMLSREDSQTCHLHKCCMFCTMQAHITWALHSPGHVIQPSQVLAAGFHRGEQEDAHEFLMFTVDAMKKACLPGHKQLDHHSKDTTLIHQIFGAYWRSQIKYLHCHGVSDTFDPYLDIALDIQAAQSVKQALEQLVKPKELNGENAYHCGLCLQKAPASKTLTLPTSAKVLILVLKRFSDVTGNKLAKNVQYPKCRDMQPYMSQQNTGPLVYVLYAVLVHAGWSCHNGHYFSYVKAQEGQWYKMDDAEVTASGITSVLSQQAYVLFYIQKSEWERHSESVSRGREPRALGAEDTDRPATQGELKRDHPCLQVPELDEHLVERATQESTLDHWKFPQEQNKTKPEFNVRKVEGTLPPNVLVIHQSKYKCGMKNHHPEQQSSLLNLSSTKPTDQESMNTGTLASLQGSTRRSKGNNKHSKRSLLVCQ

SEQ ID NO: 6 - protein, 530 aa, Homo sapiens
MGDDSLYLGGEWQFNHFSKLTSSRPDAAFAEIQRTSLPEKSPLSSETRVDLCDDLAPVARQLAPREKLPLSSRRPAAVGAGLQNMGNTCYENASLQCLTYTLPLANYMLSREHSQTCQRPKCCMLCTMQAHITWALHSPGHVIQPSQALAAGFHRGKQEDVHEFLMFTVDAMKKACLPGHKQVDHHSKDTTLIHQIFGGCWRSQIKCLHCHGISDTFDPYLDIALDIQAAQSVKQALEQLVKPEELNGENAYHCGLCLQRAPASNTLTLHTSAKVLILVLKRFSDVAGNKLAKNVQYPECLDMQPYMSQQNTGPLVYVLYAVLVHAGWSCHDGYYFSYVKAQEGQWYKMDDAEVTVCSITSVLSQQAYVLFYIQKSEWERHSESVSRGREPRALGAEDTDRPATQGELKRDHPCLQVPELDEHLVERATEESTLDHWKFPQEQNKMKPEFNVRKVEGTLPPNVLVIHQSKYKCGMKNHHPEQQSSLLNLSSMNSTDQESMNTGTLASLQGRTRRSKGKNKHSKRSLLVCQ

SEQ ID NO: 7 - protein, 526 aa, Mus musculus
MVVALSFPEADPALSSPDAPELHQDEAQVVEELTVNGKHSLSWESPQGPGCGLQNTGNSCYLNAALQCLTHTPPLADYMLSQEHSQTCCSPEGCKLCAMEALVTQSLLHSHSGDVMKPSHILTSAFHKHQQEDAHEFLMFTLETMHESCLQVHRQSKPTSEDSSPIHDIFGGWWRSQIKCLLCQGTSDTYDRFLDIPLDISSAQSVKQALWDTEKSEELCGDNAYYCGKCRQKMPASKTLHVHIAPKVLMVVLNRFSAFTGNKLDRKVSYPEFLDLKPYLSEPTGGPLPYALYAVLVHDGATSHSGHYFCCVKAGHGKWYKMDDTKVTRCDVTSVLNENAYVLFYVQQANLKQVSIDMPEGRINEVLDPEYQLKKSRRKKHKKKSPFTEDLGEPCENRDKRAIKETSLGKGKVLQEVNHKKAGQKHGNTKLMPQKQNHQKAGQNLRNTEVELDLPADAIVIHQPRSTANWGRDSPDKENQPLHNADRLLTSQGPVNTWQLCRQEGRRRSKKGQNKNKQGQRLLLVC

SEQ ID NO: 8 - protein, 467 aa, Mus musculus
MVVALSFPEDPAMSPPSAPELHQDEAQVVEELAANGKHSLSWESPQGPGCGLQNTGNSCYLNAALQCLTHTPPLADYMLSQEHSQTCCSPEGCKMCAMEAHVTQSLLHTHSGDVMKPSQILTSAFHKRKQEDAHEFLMFTLETMHESCLQVHRQSEPTSEDSSPIHDIFGGWWRSQIKCHHCQGTSYSYDPFLDIPLDISSVQSVKQALQDTEKAEELCGENSYYCGRCRQKKPASKTLKLYSAPKVLMLVLKRFSGSMGKKLDRKVSYPEFLDLKPYLSQPTGGPLPYALYAVLVHEGATCHSGHYFCCVKAGHGKWYKMDDTKVTSCDVTSVLNENAYVLFYVQQNDLKKGSINMPEGRIHEVLDAEYQLKKSGEKKHNKSPCTEDAGEPCENREKRSSKETSLGEGKVLQEQDHQKAGQKQENTKLTPQEQNHQKGGQNLRNTEGELDRLSGAIVVYQPICTAN

SEQ ID NO: 9 - protein, 545 aa, Mus musculus
MVVSLSFPEADPALSSPGAQQLHQDEAQVVVELTANDKPSLSWECPQGPGCGLQNTGNSCYLNAALQCLTHTPPLADYMLSQEYSQTCCSPEGCKMCAMEAHVTQSLLHSHSGDVMKPSQILTSAFHKHQQEDAHEFLMFTLETMHESCLQVHRQSEPTSEDSSPIHDIFGGLWRSQIKCLHCQGTSDTYDRFLDVPLDISSAQSVNQALWDTEKSEELRGENAYYCGRCRQKMPASKTLHIHSAPKVLLLVLKRFSAFMGNKLDRKVSYPEFLDLKPYLSQPTGGPLPYALYAVLVHEGATCHSGHYFSYVKARHGAWYKMDDTKVTSCDVTSVLNENAYVLFYVQQTDLKQVSIDMPEGRVHEVLDPEYQLKKSRRKKHKKKSPCTEDAGEPCKNREKRATKETSLGEGKVLQEKNHKKAGQKHENTKLVPQEQNHQKLGQKHRINEILPQEQNHQKAGQSLRNTEGELDLPADAIVIHLLRSTENWGRDAPDKENQPWHNADRLLTSQDPVNTGQLCRQEGRRRSKKGKNKNKQGQRLLLVC

SEQ ID NO: 10 - protein, 545 aa, Mus musculus
MVVSLSFPEADPALSSPGAQQLHQDEAQVVVELTANDKPSLSWECPQGPGCGLQNTGNSCYLNAALQCLTHTPPLADYMLSQEYSQTCCSPEGCKMCAMEAHVTQSLLHSHSGDVMKPSQILTSAFHKHQQEDAHEFLMFTLETMHESCLQVHRQSEPTSEDSSPIHDIFGGLWRSQIKCLHCQGTSDTYDRFLDVPLDISSAQSVNQALWDTEKSEELRGENAYYCGRCRQKMPASKTLHIHSAPKVLLLVLKRFSASMGNKLDRKVSYPEFLDLKPYLSQPTGGPLPYALYAVLVHEGATCHSGHYFSYVKAGHGKWYKMDDTKVTSCDVTSVLNENAYVLFYVQQTDLKECSIDMPEGRIHEVLDPEYQLKKSRRKKHKKKSPCTEDVGEPSKNREKKATKETSLGEGKVLQEKNHKKAGQKHENTKLVPQEQNHQKLGQKHRNNEILPQEQNHQKTGQSLRNTEGELDSPADAIVIHLPRSIANWGRDTPDKVNQPWHNADRLLTSQDLVNTGLLCRQEGRRRSKKGKNKNNQGQKLLLVR

SEQ ID NO: 11 - protein, 549 aa, Rattus Norvegicus
  misc_feature (40)..(40): Xaa can be any naturally occurring amino acid
MQSDFTASEGDRAGIKKVFAIFWRESLPSAHLENSSRLFXDDHRNMVTAHSFTEEDPAMSPPATPELHQDEARVLEELSAKGKPSLSLQRIQSPGSGLQNIGNSCYLNAVLQCLTHTPPLADYMLSQEHSQRCCYPEGCKMCAMEAHVTQSLLHSHSGGVMKPSEILTSTFHKHRQEDAHEFLMFTLNAMHESCLRGCKQSETSSKDSSLIYDIFGGQMRSQIKCHHCQGTLDSYDPFLNLFLDICSAQSVKQALEDLVKLEELQGDNAYYCGRCREKMPASKTTKVQTASKVLLLVLNRSYDFGGDKLNRVVSYPEYLDLQPYLSQPTAGPLPYALYAVLVHDGVTCSSGHYFCYVKASHGKWYKMDDSKVTRCDVSSVLSEPAYLLFYVQQTDLEKVNVDVSVGRVHGVLHPESQQKKTRKKKHKRSSCTEAVHMPRENRENTATKETSLGEGKVLQEQNHQKAGQNLKTTKVNLSANGTVIHQPRYTANWGRNAPDKDDQPGHSGDRLLTTQGSMNTGQLCGHGGSQRSKKRKNKNKQGQRPLLVC

SEQ ID NO: 12 - protein, 468 aa, Microtus ochrogaster
MAAPAAPDLRPDEGLVVAELAARAKPRMSWERIHSVGAGLQNTGNSCYLNAALQCLTHTPPLANYMLSREHSQSCGHQGGCPMCAMEAHVTQSFRHSGEVMQPSKKLTGAFHKHKQEDAHEFLMFTLNAMHESCLRGSKYSGAPSENSTPIHAIFGGSWRSQIKCLHCQGTSDSFNPFLDISLDIHAAQSVKQALEDLVQAEVLCGENAYHCDHCQGKTTASKTLMVQTAPKVLMLVLNRFSGFTGDKVDRKVSYPESLDMRPYMTQPNRGPSVYVLYAVLVHAGLTCHSGHYFCYVRAGNGKWYKMDDSKVARCDVTSVLSEPAYVLLYVRETELQKDSVTGPVDTVGQDRQRKLNRGSCVGAAEPRRPVESAAAKEISLDQWKALLEHTRPNPALNLRKTESTLPVDAVVIHQPRHRGHWDTNGPDKENYPCHTSTRLLPAQRAMGTQGGRSRTKKNKQRWRSLVV

SEQ ID NO: 13 - protein, 444 aa, Mesocricetus auratus
MDVSVDPALSSPDQPDLPQEEAQVVPELAVREEHRLSWKRPHGVGAGLENTGNSCYLNAALQCLTHTPPLASYMLSREHSQNCCHRGACMMCAMEAHVTQSFLYSGDVIQPSEMLTAAFHKHREEDAHEFLMFTLNAMHTSCLPGSKLMGCTSKQSSIIHEIFGGSWESKIKCLCCQATTDTLEPFLDITLDIQTAQSVNQALENLVKEEKLCGENAYHCDICWKNTPASKTLIVKDAPQVLLLVLNRFEEFTGDKKDREVSYSEFLDFQPYVSQSPRDPLLYVLYAVLVHDGMTCHSGHYFCYVRAGNGHWYKMNDSSVTRCDMKSVLSEPAYVLFYVQQTELKKNLWMLPQAEHQAGESRHTTINRGSPTEAEEAPDHIENTTVQDFLGHWKAPKPLTDWRKNHLDRENSPIRLLPGFCLSHQETMDTGQLCSKGERPRSKK

SEQ ID NO: 14 - protein, 482 aa, Cricetulus griseus
MKKRRRHLQEGKDPSDHSQHSRTISGSNPEDMEAARELSVGESHSKSLSVYMASTKAVGTEVYLSSCPATDPTLSSPDEPNRPQNEAQVVPELAAKEEFHLSWQRPHDVGAGLENTGNSCYMNAVLQCLTHTPPLVNYMLSREHSQNCCHQGDCMICAMEAHVTRSLLYSGDVIQPSEKLTAAFHKHRQEDAHEFLLFTLNAMHTSCLPGSKLLGCTSEQSSLIHEIFGGSWKSQIKCLHCNETTDLLEPFLDITLDIQTAQSVNQALENLVMEEQLCGENAYHCDNCRQKTMASKTLTVKDAPKVLLLVLNRFSEFTGDKKDRKVSYPESFDFQPYISQSHRQPLFYSLYAVLVHDGVTCHSGHYFCYVKAGNGHWYKMDDSSVTRCDVNSVLSEPAYVLFYVQQTDLRTNLWVLSQAEHQVGESWYTTINRGSPTEAAEPPDHTENTAAKNFLDHWKTLLNMNTKAFGETWKTQTYSESK

SEQ ID NO: 15 - protein, 529 aa, Nomascus leucogenys
MEHDSLYSGGEWHFSRFSKLTSSRPHAAFAEIQRTSLPEKSPLSSETRVDPCDDLAPVARQLAPREKLPLSSRGPAAVGAGLQNMGNTCYVNASLQCLTYTPPLANYMLSREHSQTCHRHKCCMLCTMQAHITRALHRPGDVIQPSQALAAGFHRGKQEDAHEFLMFTVDAMRKACLPGHKQVDPHSKDTTLIHQIFGGYWRSQIKCLHCQGISDTFDPYLDIALDIQAAQSVKQALEQLVKPEELNGENAYHCGLCLQKAPASKTLTLHTSAKVLILVLKRFSDVTGNKLAKNVQYPECLDMQPYMSQQNTGPLVYVLYAVLVHAGWSCHNGHYFSYVKAQEGQWYKMDDAEVTASGITSVLSQQAYVLFYIQKSELERHSEGVSRGREPRALGPADTDRRATQGELKREPCLQVPELDEHSVERATQESTLDHWKFLQEQNKTKPEFNVRKVEGSLPPNVVVIHQSKYKCGTKNHHPEQQSSLLNLSSTNPTDQESINTGTPASRQGRTRRSKGKNKHSNRALLLCQ

SEQ ID NO: 16 - protein, 530 aa, Pan troglodytes
MEDDSLYLGGEWQFNHFSKLTSSRPDAAFAEIQRTSLPEKSPLSSETRVDLCDDLAPVARQLAPGEKLLLSSRRPAAVGAGLQNMGNTCYVNASLQCLTYTPPLANYMLSREHSQTCHRHKGCMLCTMQAHITRALHIPGHVIQPSQALAAGFHRGKQEDAHEFLMFTVDAMEKACLPGHKQVEHHSKDTTLIHQIFGGYWRSQIKCLHCHGISDTFDPYLDIALDIQAAQSVQQALEQLVKPEELNGENAYHCGLCLQRAPASKTLTLHTSAKVLILVLKRFSDVTGNKLAKNVQYPECLDMQPYMSQQNTGPLVYVLYAVLVHAGWSCHNGHYFSYVKAQEGQWYKMDDAEVTASSITSVLSQQAYVLFYIQKSEWERHSESASRGREPRALGAEDTDRRATQGELKRDHPCLQAPELGEHLVERATQESTLDHWKFLQEQNKTKPEFNVRKVEGTLPPNVLVIHQSKYKCGMKNHHPEQQSSLLNLSSTTPTDQESMNTGTLASLQGRTRRSKGKNKHSKRALLVCQ

SEQ ID NO: 17 - protein, 530 aa, Macaca mulatta
MEDDSLYLGGEWQFNHFSKLTSSRPDAAFAEIQRTSLPEKSPLSSETRVDLCDDLAPVARQLAPREKLPLSSRRPAAVGAGLQNMGNTCYVNASLQCLTYTPPLANYMLSREHSPTCHRHKGCMLCTMQAHITRALHIPGRVIQPSQALAADFHRGKQEDAHEFLMFTVDAMKKACLPGHKQVDHHSKDTTLIHQIFGGYWRSQIKCLHCHGISDTFDPYLDIALDIQAAQSVKQALEQLVKPEELNGENAYHCGLCLQRAPASKTLTLHTSAKVLILVLKRFSDVTGSKLAKNVHYPECLDMQPYMSQQNTGPLVYVLYAVLVHAGWSCHNGHYFSYVKAQEGQWYKMDDAEVTASSITSVLSQQAYVLFYIQKSEWERHSESASRGREPRALGAEDTDRRATQGELKRDHPCLQAPELDEHLVERATQESTLDHWKFLQEQNKTKPEFNVRKVEGTLPPNVLVIHQSKYKCGMKNHHPEQQSSPLNLSSTTPTDQESVNTGTLASLQGRTRRSKGKNKHSKRALLVCQ

SEQ ID NO: 18 - protein, 533 aa, Bos Taurus
METLVGLVCRAPGAASEHGGGVCPSPFDVFPGGQGCGPSAAGADALRGPSVPEGPSPALGRPQRGDLAPGSAGLTPGQKGALSWKGPWGVGAGLQNLGNTCYVNAALQCLSHTPPLASWMVSQQHATLCPARSACTLCAMRAHVTRALLHAGEVIRPRKDLLAGFHRHQQEDAHEFLMFTLNAMQQGCLSASQPSGHASEDTTVIRQIFGGTWRSQIQCLRCLGVSDTFDPYLDISLDITAAQSVEQALRELVKPEKLDADNAYDCGVCLRKVPATKRLTLHSTSQVLVLVLKRFTPVSGAKRAQEVRYPQCLDLQPYTSERKAGPLGYVLYAVLVHSGWSCERGHYFCYVRAGNGQWYKMDDAKVTACDETAALSQSAYVLFYAREGAWEGGAGGGAAAPVGADPTEPGQPAGDASGRAPGSEESPGDTEVEGMSLEQWRRLQEHSRPKPALELRKVQSALPAGAVVIHQSKHGGGRNRTPPQQEHERLDRPSTDTPPPGPKNVGNGPCAGGRARATKGKNKKPRPSLGLWR

SEQ ID NO: 19 - protein, 535 aa, Canis lupus familiaris
MEAAHLHPSEEPQFSASPKPQSYWSRGGGAEVHGGPSVPETTSPASKTLSSPTDPLAPTSAGLPPTKTPLSWRSLSQVGAGLQNMGNTCYVNATLQCLTYTEPLASYMLSQQHGTTCRRQTSCMLCTLQAHLTRVLCHPGRVLRPLPLLLAAFHRHKQEDAHEYLMFILDAMQQACLPEDKLSDPECPQDSTLIQQLFGGYWRSQIQCLHCQGISSTLEPYLDISLDIGAAHSISQALEQLMKPELLEGENAYHCSKCLEKVPASKVLTLHTSPKVLILVLRRFSDLTGNKMTKEVQYPERLDMQHYLSEQRAGPLVYVLYAVLVHAGRSCHSGHYFCFVKAGNGQWYKMDDAKVSACDVTCALRQPAYVLFYMQKTDLERDLGRESVEEGGLASPEADPTVVGEASGEPATDPSGNHPELEERGEETSRQQMTLDQWRCLQECNRPKPELHVRRREIALPANAVILHHSKYRPEMPKNHPQPTVDLLTTAAGMLPPQVAGDMAKVPRVPGRARPTKRTSKKGQRSGEAVQGCVS

SEQ ID NO: 20 - DNA, 1593 bp, Homo sapiens
atggaggacgactcactctacttgggaggtgagtggcagttcaaccacttttcaaaactcacatcttctcggccagatgcagcttttgctgaaatccagcggacttctctccctgagaagtcaccactctcatgtgagacccgtgtcgacctctgtgatgatttggctcctgtggcaagacagcttgctcccagggagaagcttcctctgagtagcaggagacctgctgcggtgggggctgggctccagaatatgggaaatacctgctacgtgaacgcttccttgcagtgcctgacatacacaccgccccttgccaactacatgctgtcccgggagcactctcaaacgtgtcatcgtcacaagggctgcatgctctgtacgatgcaagctcacatcacacgggccctccacaatcctggccacgtcatccagccctcacaggcattggctgctggcttccatagaggcaagcaggaagatgcccatgaatttctcatgttcactgtggatgccatgaaaaaggcatgccttcccgggcacaagcaggtggatcatcactctaaggacaccaccctcatccaccaaatatttggaggctactggagatctcaaatcaagtgtctccactgccacggcatttcagacacttttgacccttacctggacatcgccctggatatccaggcagctcagagtgtccagcaagctttggaacagttggtgaagcccgaagaactcaatggagagaatgcctatcattgtggtgtttgtctccagagggcgccggcctccaagacgttaactttacacacctctgccaaggtcctcatccttgtattgaagagattctccgatgtcacaggcaacaagattgccaagaatgtgcaatatcctgagtgccttgacatgcagccatacatgtctcagccgaacacaggacctctcgtctatgtcctctatgctgtgctggtccacgctgggtggagttgtcacaacggacattacttctcttatgtcaaagctcaagaaggccagtggtataaaatggatgatgccgaggtcaccgcctctagcatcatttctgtcctgagtcaacaggcctacgtcctcttttacatccagaagagtgaatgggaaagacacagtgagagtgtgtcaagaggcagggaaccaagtgcccttggcgcagaagacacagacaggcgagcaacgcaaggagagctcaagagagaccacccctgcctccaggcccccgagttggacgagcacttggtggaaagagccactcaggaaagcaccttagaccactggaaattccttcaagagcaaaacaaaacgaagcctgagttcaacgtcagaaaagtcgaaggtaccctgcctcccgacgtacttgtgattcatcaatcaaaatacaagtgtgggatgaagaaccatcatcctgaacagcaaagctccctgctaaacctctcttcgacgaccccgacacatcaggagtccatgaacactggcacactcgcttccctgcgagggagggccaggagatccaaagggaagaacaaacacagcaagagggctctgcttgtgtgccagtga

SEQ ID NO: 21 - DNA, 1885 bp, Mus musculus
atggtggtttctctttccttcccagaagagactggaggggaaaatcttccttctgctcccttagaagactccagcaagttctttgaagaggtctttggagacatggtggttgctctttccttcccagaagcagatccagcactatcatctcctgatgccccagagctgcatcaggatgaagctcaggtggtggaggagctaactaccaatggaaagcacagtctgagttgggagagtccccaaggaccaggatgcgggctccagaacacaggcaacagctgctacctgaacgcagccctgcagtgcttgacacacacaccacctctagctgactacatgctgtcccaggagcacagtcaaacctgttgttccccagaaggctgtaagatgtgtgctatggaagcccatgtgacccagagtctcctgcactctcactcgggggatgtcatgaagccctcccagattttgacctctgccttccacaagcaccagcaggaagatgcccatgagtttctcatgttcaccttggaaacaatgcatgaatcctgccttcaagtgcacagacaatcagatcccacccctcaggatacgtcacccattcatgacatatttggaggctggtggaggtctcagatcaagtgtctccattgccagggcacctcacataccttcgatcccttcctggatgtccccctggatatcagctcagctcagagtgtaaatcaagccttgtgggatacagggaagtcagaagagctacttggagagaatgcctactactgtggtaggtgtagacagaagatgccagcttctaagaccctgcatgttcatattgctccaaaggtactcctgctagtgttaaagcgcttctcagccttcacgggtaacaagttagacagaaaagtaagctacccggagttccttgacctgaagccatacctgtctgagcctactggaggacctttgccttatgccctctatgccgtcctggtccatgatggtgcgacttctaacagtggacattacttctgttgtgtcaaagctggtcatgggaagtggtacaagatggatgatactaaggtcaccaggtgtgatgtgacttctgtcctgaatgagaatgcctatgtgctcttctatgtgcagcagaccgacctcaaacaggtcagtattgacatgccagagggcagagtacatgaggttcttgaccctaaataccagctgaagaaatcccggagaaaaaagcgtaagaagcaatgccattgcacagatgatgccggagaggcatgcgaaaacagggagaagagagcaaagaaagaaacctccttaggggaggggaaagtgcctcaggaagtgaaccacgagaaagctgggcagaaacatgggaataccaaactcgtgcctcaggaacagaaccaccagagagctgggcagaacctcaggaatactgaagttgaacttgatctgcctgttgatgcaattgtgattcaccagcccagatccacagcaaactggggcacggatgctccagacaaagagaatcaaccctggcacaatggtgacaggctcctcacctctcagggcctcatgagccctgggcagctctgtagtcagggtgggagatgaagatcgaagaaggggaagaacaagaacaagcaagggcagaggcttctgcttgtttgctagtgctcacccacccactcacacaggctcctgtggacacactgttgacccaaggtgcctggaacaagaggtttggatctctgtttcaggcagggacaatgccttacccttcacgtggggtccacatttcctctgggtccttgcctgtttttgctgactgactctctgatcgtttgaatgtggaaaaaatgcccaggatgttggt

SEQ ID NO: 22 - DNA, 1593 bp, Homo sapiens
atggaggacgactcactctacttgggaggtgagtggcagttcaaccacttttcaaaactcacatctcctcggcccgatgcagcttttgctgaaatccagcggacttctctccctgagaagtcaccactctcatgtgagacccgtgtcgacctctgtgatgatttggctcctgtggcaaggcagcttgctcccagggagaagcttcctctgagtagcaggagacctgctgcggtgggggctgggcttcagaatatgggaaatacctgctacgtgaacgcttccttgcagtgcctgacatacacaccgccccttgccaactacatgctgtcccgggagcactctcaaacgtgtcatcgtcacaagggctgcatgctctgtactatgcaagctcacatcacacgggccctccacaatcctggccacgtcatccagccctcacaggcattggctgctggcttccatagaggcaagcaggaagatgcccatgaatttctcatgttcactgtggatgccatgaaaaaggcatgccttcccgggcacaagcaggtggatcatcactctaaggacaccaccctcatccaccaaatatttggaggctactggagatctcaaatcaagtgtctccactgccacggcatttcagacacttttgacccttacctggacatcgccctggatatccaggcagctcagagtgtccagcaagctttggaacagttggtgaagcccgaagaactcaatggagagaatgcctatcattgtggtgtttgtctccagagggcgccggcctccaagacgttaactttacacacctctgccaaggtcctcatccttgtattgaagagattctccgatgtcacaggcaacaagattgccaagaatgtgcaatatcctgagtgccttgacatgcagccatacatgtctcagcagaacacaggacctcttgtctatgtcctctatgctgtgctggtccacgctgggtggagttgtcacaacggacattacttctcttatgtcaaagctcaagaaggccagtggtataaaatggatgatgccgaggtcaccgcctctagcatcactcctgtcctgactcaacaggcctacgtcctcttttacatccagaagagtgaatgggaaagacacagtgagagtgtgtcaagaggcagggaaccaagagcccttggcgcagaagccacagacaggcgagcaacgcaaggagagctcaagagagaccacccctgcctccaggcccccgagttggacgagcacttggtggaaagagccactcacgaaagcaccttagaccactggaaattccttcaagagcaaaacaaaacgaagcctgagttcaacgtcagaaaagtcgaaggtaccctgcctcccgacgtacttgtgattcatcaatcaaaatacaagtgtgggatgaagaaccatcatcctgaacagcaaagctccctgctaaacctctcttcgacgaccccgacacatcaggagtccatgaacactggcacactcgcttccctgcgagggggggccaggagatccaaagggaagaacaaacacagcaagagggctctgcttgtgtgccagtga

SEQ ID NO: 23 - DNA, 1593 bp, Homo sapiens
atgggggatgactcactctacttgggaggtgagtggcagttcaaccacttttcaaaactcacatcttctcggccagatgcagcttttgctgaaatccagcggacttctctccctgagaagtcaccactctcatctgagacccgtgtcgacctctgtgatgatttggctcctgtggcaagacagctcgctcccagggagaagcttcctctgagtagcaggagacctgctgcggtgggggctgggctccagaatatgggaaatacctgctacgagaacgcttccctgcagtgcctgacatacacactgccccttgccaactacatgctgtcccgggagcactctcaaacatgtcagcgtcccaagtgctgcatgctctgtactatgcaagctcacatcacatgggccctccacagtcctggccatgtcatccagccctcacaggcattggcttctggcttccatagaggcaagcaggaagatgtccatgaatttctcatgttcactgtggatgccatgaaaaaggcatgccttcccggccacaagcaggtagatcatcactctaaggacaccaccctcatccaccaaatatttggaggctgctggagatctcaaatcaagtgtctccactgccacgggatttcagacacttttgacccttacctggacatcgccctggatatccaggcagctcagagtgtcaagcaagctttggaacagttggtgaagcccgaagaactcaatggagagaatgcctatcattgcggtctttgtctccagagggcgccggcctccaacacgttaactttacacacttctgccaaggtcctcatccttgtcttgaagagattctccgatgtcgcaggcaacaaacttgccaagaatgtgcaatatcctgagtgccttgacatgcagccatacatgtctcagcagaacacaggacctcttgtctatgtcctctatgctgtgctggtccacgctgggtggagttgtcacgacggacattacttctcttatgtcaaagctcaagaaggccagtggtataaaatggatgatgccgaggtcactgtctgtagcatcacttctgtcctgagtcaacaggcctatgtcctcttttacatccagaagagtgaatgggaaagacacagtgagagtgtgtcaagaggcagggaaccaagagccctcggcgctgaagacacagacaggcgagcaaagcaaggagagctcaagagagaccacccctgcctccaggcacccgagttggacgagcacttggtggaaagagccactcaggaaagcaccttagaccactggaaattcctccaagagcaaaacaaaacgaagcctgagttcaacgtcggaaaagtcgaaggtaccctgcctcccaacgcacttgtgattcatcaatcaaaatacaagtgtgggatgaaaaaccatcatcctgaacagcaaagctccctgctaaacctctcttcgacgacccggacagatcaggagtccatgaacactggcacactcgcttctctgcaagggaggaccaggagagccaaagggaagaacaaacacagcaagagggctctgcttgtgtgccagtga

SEQ ID NO: 24 - DNA, 1593 bp, Homo sapiens
atggaagacgactcactctatttgggaggtgactggcagttcaatcacttttcaaaactcacatcttctcggctagatgcagcttttgctgaaatccagcggacttctctctctgaaaagtcaccactctcatctgagacccgtttcgacctctgtgatgatttggctcctgtggcaagacagcttgctcccagggagaagcttcctctgagtagcaggagacctgctgcggtgggggctgggctccagaagataggaaataccttctatgtgaacgtttccctgcagtgcctgacatacacactgccgctttccaactacatgctgtcccgggaggactctcaaacgtgtcatcttcacaagtgctgcatgttctgtactatgcaagctcacatcacatgggccctccacagtcctggccatgtcatccagccctcacaggtattggctgctggcttccatagaggtgagcaggaggatgcccatgaatttctcatgtttactgtggatgccatgaaaaaggcatgccttcccgggcacaagcagctagatcatcactccaaggacaccaccctcatccaccaaatatttggagcgtattggagatctcaaatcaagtatctccactgccacggcgtttcagacacctttgacccttacctggacatcgccctggatatccaggcagctcagagtgtcaagcaagctttggaacagttggtgaagcccaaagaactcaatggagagaatgcctatcattgtggtctttgtctccagaaggcgcctgcctccaagacgttaactttacccacttctgccaaggtcctcattcttgtattgaagagattctccgatgtcacaggcaacaaacttgccaagaatgtgcaatatcctaagtgccgtgacatgcagccatacatgtctcagcagaacacaggacctcttgtctatgtcctctatgctgtgctggtccacgctgggtggagttgtcacaacggacattacttctcttatgtcaaagctcaagaaggccagtggtataaaatggatgatgccgaggtcactgcctctggcatcacctctgtcctgagtcaacaggcctatgtcctcttttacatccagaagagtgaatgggaaagacacagtgagagtgtgtcaagaggcagggaaccaagagcccttggtgctgaagacacagacaggccagcaacgcaaggagagctcaagagagaccacccttgcctccaggtacccgagttggacgagcacttggtggaaagagccactcaggaaagcaccttagaccactggaaattcccccaagagcaaaacaaaacgaagcctgagttcaacgtcagaaaagttgaaggtaccctgcctcccaacgtacttgtgattcatcaatcaaaatacaagtgtggtatgaaaaaccatcatcctgaacagcaaagctccctgctaaacctctcttcgacgaaaccgacagatcaggagtccatgaacactggcacactcgcttctctgcaagggagcaccaggagatccaaagggaataacaaacacagcaagagatctctgcttgtgtgccagtga

SEQ ID NO: 25 - DNA, 1593 bp, Homo sapiens
atgggggacgactcactctacttgggaggtgagtggcagttcaaccacttttcaaaactcacatcttctcggccagatgcagcttttgctgaaatccagcggacttctctccctgagaagtcaccactctcatctgagacccgtgtcgacctctgtgatgatttggctcctgtggcaagacagctcgctcccagggagaagcttcctctgagtagcaggagacctgctgcggtgggggctgggctccagaatatgggaaatacctgctacgagaacgcttccctgcagtgcctgacatacacactgccccttgccaactacatgctgtcccgggagcactctcaaacatgtcagcgtcccaagtgctgcatgctctgtactatgcaagctcacatcacatgggccctccacagtcctggccatgtcatccagccctcacaggcattggctgctggcttccatagaggcaagcaggaagatgtccatgaatttctcatgttcactgtggatgccatgaaaaaggcatgccttcccggccacaagcaggtagatcatcactctaaggacaccaccctcatccaccaaatatttggaggctgctggagatctcaaatcaagtgtctccactgccacgggatttcagacacttttgacccttacctggacatcgccctggatatccaggcagctcagagtgtcaagcaagctttggaacagttggtgaagcccgaagaactcaatggagagaatgcctatcattgcggtctttgtctccagagggcgccggcctccaacacgttaactttacacacttctgccaaggtcctcatccttgtcttgaagagattctccgatgtcgcaggcaacaaacttgccaagaatgtgcaatatcctgagtgccttgacatgcagccatacatgtctcagcagaacacaggacctcttgtctatgtcctctatgctgtgctggtccacgctgggtggagttgtcacgacggatattacttctcttatgtcaaagctcaagaaggccagtggtataaaatggatgatgccgaggtcactgtctgtagcatcacttctgtcctgagtcaacaggcctatgtcctcttttacatccagaagagtgaatgggaaagacacagtgagagtgtgtcaagaggcagggaaccaagagcccttggcgctgaagacacagacaggccagcaacgcaaggagagctcaagagagaccacccttgcctccaggtacccgagttggacgagcacttggtggaaagagccactgaggaaagcaccttagaccactggaaattcccccaagagcaaaacaaaatgaagcctgagttcaacgtcagaaaagttgaaggtaccctgcctcccaacgtacttgtgattcatcaatcaaaatacaagtgtgggatgaaaaaccaccatcctgaacagcaaagctccctgctaaacctctcttcgatgaactcgacagatcaggagtccatgaacactggcacactcgcttctctgcaagggaggaccaggagatccaaagggaagaacaaacacagcaagagatctctgcttgtgtgccagtga

SEQ ID NO: 26 - DNA, 2661 bp, Mus musculus
aggaaaaacttccttctgctcccttagaagactccagctagttatttgaagaggtctttgtagacacggtggttgctctttcctcccaagaagagattctctagaagggaaaaacttccttctgctcccttagaagactacagcaagttctttgaagaggtctttggagacatggtggttgctctttccttcccagaagcagatccagccctatcatctcctgatgccccagagctgcatcaggatgaagctcaggtggtggaggagctaactgtcaatggaaagcacagtctgagttgggagagtccccaaggaccaggatgcgggctccagaacacaggcaacagctgctacctgaatgcagccctgcagtgcttgacacacacaccacctctagctgactacatgctgtcccaggagcacagtcaaacctgttgttccccagaaggctgtaagttgtgtgctatggaagcccttgtgacccagagtctcctgcactctcactcgggggatgtcatgaagccctcccatattttgacctctgccttccacaagcaccagcaggaagatgcccacgagtttctcatgttcaccttggaaacaatgcatgaatcctgccttcaagtgcacagacaatcaaaacccacctctgaggacagctcacccattcatgacatatttggaggctggtggaggtctcagatcaagtgtctcctttgccagggtacctcagatacctatgatcgcttcctggacatccccctggatatcagctcagctcagagtgtaaagcaagccttgtgggatacagagaagtcagaagagctatgtggagataatgcctactactgtggtaagtgtagacagaagatgccagcttctaagaccctgcatgttcatattgctccaaaggtactcatggtagtgttaaatcgcttctcagccttcacgggtaacaagttagacagaaaagtaagttacccggagttccttgacctgaagccatacctgtctgagcctactggaggacctttgccttatgccctctatgccgtcctggtccatgatggtgcgacttctcacagtggacattacttctgttgtgtcaaagctggtcatgggaagtggtacaagatggatgatactaaagtcaccaggtgtgatgtgacttctgtcctgaatgagaatgcctatgtgctcttctatgtgcagcaggccaacctcaaacaggtcagtattgacatgccagagggaagaataaatgaggttcttgaccctgaataccagctgaagaaatcacggagaaaaaagcataagaagaaaagccctttcacagaagatttaggagagccctgcgaaaacagggataagagagcaattaaagaaacctccttaggaaaggggaaagtgcttcaggaagtgaaccacaagaaagctgggcagaaacacgggaataccaaactcatgcctcagaaacagaaccaccagaaagctgggcagaacctcaggaatactgaagttgaacttgatctgcctgctgatgcaattgtgattcaccagcccagatccactgcaaactggggcagggattctccagacaaggagaatcaacccttgcacaatgctgacaggctcctcacctctcagggccctgtgaacacttggcagctctgtagacaggaagggagacgaagatcgaagaaggggcagaacaagaacaagcaagggcagaggcttctgcttgtttgctagtgatcacccacccactcacacaggctcctgtggacacactgttgacccaaggtgcctggaacaagaggtttggatctctgtttcaggcagggacaatgcctcacccttcacgtggggtccacctatcctctgggcccttgcctgtttttgctgactgactctctgattgtttgaatgtggaaaaaaagtgcccaggatgttggtacaggttaaagacaagaagctggacacccggaggaggtctgaatagcctctcctgcaactcatggaatctgagcagcatagagactaaatcaccacactggagctttcttttcttttcttttcttttcttttcttttcttttcttttcttttctcttctcttctcttctcttctcttctcttctcttctcttctcttctcttctcttctcttctctcctctcctctcctctcctctcctctcctctcctctcctctcctttcctttcctttcctttcctttttttttaaatttattttttgttattagatattttctttatttacatttcaaatgctatcccaaaagttccctataccctcccccaactctgccaccctacccacccactcccacttcttggctctggcatttccctgtactggggcatataaagtttgcaataccaaagggcctctcttcccaatgatggccaactaggtcaccttctgctacatatgcagctagagaccctaagaaaacacactggaactcttgaggtttggagttttcgctcaggcaacaagttgcttttcaactgccctttctaacctcttacccagaaaatgtgtagttcaccctgtagagatagatgctcttattcttagtgtgtgatcaacagttctttggtcaaataaattctgttacttcacaaaaaaaaaaaaaa

SEQ ID NO: 27 - DNA, 1407 bp, Mus musculus
atggtggttgctctctccttcccagaagcagatccagccatgtcacctcctagtgccccagagctgcatcaggatgaagcccaggtggtagaggagctggctgccaatggaaagcacagtctgagttgggagagtccccaaggaccaggatgcgggctccagaacacaggcaacagctgctacctgaatgcagccctgcagtgcttgacacacacaccacctctagctgactacatgctgtcccaggagcacagtcaaacctgttgttccccagaaggctgtaagatgtgtgctatggaagcccatgtgacccagagtctcctgcacacccactcaggggatgttatgaagccctcccagaatttgacctctgccttccacaagcgcaagcaggaagatgcccatgagtttctcatgttcaccttggaaacaatgcatgaatcctgccttcaagtgcacagacaatcagaacccacctctgaggacagctcacccattcatgacatatttggaggctggtggaggtctcagatcaagtgtcaccactgccagggcacctcatattcctatgatcccttcctggacatccccctggatatcagctcagttcagagtgtgaagcaagccttgcaggatacagagaaggcagaagagctatgtggggagaattcctactactgtggtaggtgtagacaaaagaagccagcttccaagaccctaaagctttatagtgccccaaaggtactcatgctagtgttaaagcgcttttcgggctctatgggtaaaaagttggacagaaaagtaagctacccagagttccttgacctgaagccatacctgtcccagcctactggagggcctttgccttatgccctctatgccgtcctggtccatgaaggtgcaacttgtcacagtggacattacttctgttgtgtcaaagctggccatgggaagtggtacaagatggatgatactaaggtcaccagctgtgatgtgacttctgtcctgaatgagaatgcctatgtgctcttctatgtgcagcagaatgacctcaaaaagggtagtatcaacatgccagagggcagaatacatgaggttcttgatgccaaataccagctgaagaaatcaggggaaaaaaagcataataaaagcccttgcacagaagatgcaggagagccctgcgaaaacagggagaagagatcatccaaagaaacctccttaggggaggggaaagttcttcaggaacaggaccaccagaaagctgggcagaaacaagagaataccaaactcacgcctcaggaacagaaccacgagaaaggtgggcagaacctcaggaatactgaaggtgaacttgatcgactcagtggtgcaattgtggtttaccaacctatatgcactgcaaactga

SEQ ID NO: 28 - DNA, 1697 bp, Mus musculus
atttgaagaggtctttggagacatggtggtttctctttccttcccagaagcagatccagccctatcatctcctggtgcccaacagctgcatcaggatgaagctcaggtagtggtggagctaactgccaatgacaagcccagtctgagttgggaatgtccccaaggaccaggatgcgggcttcagaacacaggcaacagctgctacctgaatgcagccctgcagtgcttgacacacacaccacctctagctgactacatgctgtcccaggagtacagtcaaacctgttgttccccagaaggctgtaagatgtgtgctatggaagcccatgtaacccagagtctcctgcactctcactcgggggatgtcatgaagccctcccagattttgacctctgccttccacaagcaccagcaggaagatgcccatgagtttctcatgttcaccttggaaacaatgcatgaatcctgccttcaagtgcacagacaatcagaacccacctctgaggacagctcacccattcatgacatatttggaggcttgtggaggtctcagatcaagtgtctccattgccagggtacctcagatacatatgatcgcttcctggatgtccccctggatatcagctcagctcagagtgtaaatcaagccttgtgggatacagagaagtcagaagagctacgtggagagaatgcctactactgtggtaggtgtagacagaagatgccagcttccaagaccctgcatattcatagtgccccaaaggtactcctgctagtgttaaagcgcttctcggccttcatgggtaacaagttggacagaaaagtaagctacccagagttccttgacctgaagccatacctgtcccagcctactggaggacctttgccttatgccctctatgctgtcctggtccatgaaggtgcgacttgtcacagtggacattacttctcttatgtcaaagccagacatggggcatggtacaagatggatgatactaaggtcaccagctgcgatgtgacttctgtcctgaatgagaatgcctatgtgctcttctatgtgcagcagactgacctcaaacaggtcagtattgacatgccagagggcagagtacatgaggttctcgaccctgaataccagctgaagaaatcccggagaaaaaagcataagaagaaaagcccttgcacagaagatgcgggagagccctgcaaaaacagggagaagagagcaaccaaagaaacctccttaggggaggggaaagtgcttcaggaaaagaaccacaagaaagctgggcagaaacatgagaataccaaacttgtgcctcaggaacagaaccaccagaaacttgggcagaaacacaggatcaatgaaatcttgcctcaggaacagaaccaccagaaagctgggcagagcctcaggaacacggaaggtgaacttgatctgcctgctgatgcaattgtgattcacctgctcagatccacagaaaactggggcagggatgctccagacaaggagaatcaaccctggcacaatgctgacaggctcctcacctctcaggaccctgtgaacactgggcagctctgtagacaggaaggaagacgaagatcaaagaaggggaagaacaagaacaagcaagggcagaggcttctgcttgtttgctagtgttcactcacccactcacacaggctcctgtggacac

SEQ ID NO: 29 - DNA, 1638 bp, Mus musculus
atggtggtttctctttccttcccagaagcagatccagccctatcatctcctggtgcccaacagctgcatcaggatgaagctcaggtagtggtggagctaactgccaatgacaagcccagtctgagttgggaatgtccccaaggaccaggatgcgggcttcagaacacaggcaacagctgctacctgaatgcagccctgcagtgcttgacacacacaccacctctagctgactacatgctgtcccaggagtacagtcaaacctgttgttccccagaaggctgtaagatgtgtgctatggaagcccatgtaacccagagtctcctgcactctcactcgggggatgtcatgaagccctcccagattttgacctctgccttccacaagcaccagcaggaagatgcccatgagtttctcatgttcaccttggaaacaatgcatgaatcctgccttcaagtgcacagacaatcagaacccacctctgaggacagctcacccattcatgacatatttggaggcttgtggaggtctcagatcaagtgtctccattgccagggtacctcagatacatatgatcgcttcctggatgtccccctggatatcagctcagctcagagtgtaaatcaagccttgtgggatacagagaagtcagaagagctacgtggagagaatgcctactactgtggtaggtgtagacagaagatgccagcttccaagaccctgcatattcatagtgccccaaaggtactcctgctagtgttaaagcgcttctcggccttcatgggtaacaagttggacagaaaagtaagctacccggagttccttgacctgaagccatacctgtcccagcctactggaggacctttgccttatgccctctatgctgtcctggtccatgaaggtgcgacttgtcacagtggacattacttctcttatgtcaaagctggacatgggaagtggtacaagatggatgatactaaggtcaccagctgcgatgtgacttctgtcctgaatgagaatgcctatgtgctcttctatgtgcagcagactgacctcaaagaggtcagtattgacatgccagagggcagaatacatgaggttctcgaccctgaataccagctgaagaaatcccggagaaaaaagcataagaagaaaagcccttgcacagaagatgtgggagagccctccaaaaacagggagaagaaagcaaccaaagaaacctccttaggggaggggaaagtgcttcaggaaaagaaccacaagaaagctgggcagaaacacgagaataccaaactcgtgcctcaggaacagaaccaccagaaactggggcagaaacacaggaacaatgaaatcttgcctcaggaacagaaccaccagaaaactgggcagagcctcaggaacacggaaggtgaacttgatctgcctgctgatgcaattgtgattcacctgcccagatccatagcaaattggggcagggatactccagacaaggtgaatcaaccctggcacaatgctgacaggctcctcacctctcaggaccttgtgaacactgggcagctctgtagacaggaaggaagacgaagatcgaagaaggggaagaacaagaacaagcaagggcagaagcttctgcttgttcgctag

SEQ ID NO: 30 - DNA, 1650 bp, Rattus Norvegicus
atgcaatctgatttcactgcttcagaaggagatagagcaggaattaagaaagtttttgcgattttctggagggaaagtcttccttctgctcacttagaaaactcaagcaggttattttaagatgatcatagaaatatggtgactgctcactccttcacagaagaagatccagccatgtcaccacctgctaccccagagctccatcaagatgaagcacgggtgctagaggagctgtctgccaaggggaagcccagtctgagtttgcagaggatccaaagcccagggtcagggctccagaacataggcaacagctgctacctgaatgcagtcctgcagtgcttgacacacacaccacctcttgccgactatatgctgtcccaggagcattctcagaggtgttgttacccagaaggctgtaagatgtgtgctatggaagctcatgtgacccagagtctcctccactcccactcaggaggtgtcatgaaaccctccgagatattgacctctaccttccacaagcacaggcaggaagatgcccatgaatttctcatgttcaccttgaatgccatgcatgaatcctgccttcgagggtgcaagcaatcagaaacctcctctaaggacagctccctgatctatgacatatttggaggccagatgagatctcagatcaagtgtcaccactgccagggcaccttagattcctacgatcccttcctgaacctcttcctggatatctgctctgctcagagtgtgaagcaagccttggaggacttagtgaagctagaagagttgcagggggacaacgcctactactgtggtaggtgtagagagaagatgccagcttctaagaccacgaaggttcagactgcctcaaaggttctcctgctagtgttaaaccgctcctacgatttcgggggtgacaagttgaacagggtagtaagctacccagagtaccttgacctgcagccatacctgtcacagccaactgcaggacccctgccttatgctctctatgccgtcctggtccatgatggggtgacttgttccagtggacattacttctgttatgtcaaagccagccatgggaagtggtataagatggatgattctaaggtcaccaggtgcgatgtgtcctctgtcctgagcgagcctgcctatctgctcttctatgtccagcagactgaccttgagaaggtcaatgttgatgtgtctgtgggcagagtacatggggttcttcaccctgaatcccagcagaagaaaacgcggaagaaaaagcacaagagaagctcttgcacagaagctgtacacatgccccgagaaaacagggaaaatacagccaccaaagaaacctccttaggggaggggaaagtgcttcaggaacagaaccaccagaaagctgggcagaacctcaagactaccaaagtcaatttgtcagccaacggaactgtgattcatcagcccagatataccgcaaactggggcaggaatgctccagacaaggacgatcaaccagggcacagtggtgacagactcctcaccactcagggctccatgaacactgggcaactctgtggtcatggagggagccaaagatctaagaagaggaagaacaagaacaagcaagggcagaggcctctgcttgtttgctag

SEQ ID NO: 31 - DNA, 1407 bp, Microtus ochrogaster
atggcagctcctgccgccccagacctgcgtccagatgaagggctagtggtagcagagctggctgccagggccaagcccagaatgagttgggagagaatccacagcgtgggtgcgggtctccagaacactggaaacagttgctaccttaatgcagccttgcagtgtctgacgcacacgccacctcttgccaactacatgctctcccgggagcactctcagagctgcggtcaccagggaggctgtccaatgtgtgccatggaagctcatgtgacccagagtttccgccactccggggaggtcatgcagccttcaaagaagctgactggagccttccacaagcacaagcaggaggatgcccacgagtttctgatgttcaccttgaacgccatgcacgaatcctgccttcgagggagcaagtactcaggagccccatctgagaacagcacccctatccacgcgatatttggaggctcatggagatctcagatcaagtgtctccactgccagggcacctcagattcctttaacccgttcctggacatatccctggatatccacgcggctcagagtgtgaagcaagccttggaggatttagtgcaggccgaagtgctgtgtggagaaaatgcctaccactgtgaccactgccaggggaagacgacagcttcaaagaccctgatggtccaaactgccccgaaggttctcatgctggtcttgaatcgcttctcaggtttcacgggcgacaaagtcgacagaaaagtgagctaccctgagtcccttgacatgcggccatacatgactcagcctaatagaggaccatcggtctatgtactctatgctgtgctggtccatgccggtttgacgtgccacagtggacattacttctgttatgtcagagctggcaatgggaagtggtataagatggacgactcgaaagtcgccaggtgtgatgtgacttctgtcctgagtgagccagcctatgtgcttctctatgttcgggagactgaactccaaaaggacagcgtcactgggccagtagacacagttggccaagaccgacagagaaagctcaacagaggatcttgtgtgggagctgcagagccacgcaggcccgtggagagcgctgcagccaaagaaatctccttagaccagtggaaagcgcttctggaacacacccgcccgaatcccgcgctgaacctcaggaaaactgagtccactctgccagttgatgctgttgttattcaccagcccagacacagagggcactgggacacaaatggtcccgacaaggagaattacccttgtcacacttcaaccaggttgctgcctgctcagagagccatgggcactcagggaggaagatccagaaccaagaagaacaagcaaaggtggaggtctctggttgtttaa

SEQ ID NO: 32 - DNA, 1335 bp, Mesocricetus auratus
atggacgtctcagtagatcctgccctgtcatctcctgatcaaccagacctgccccaggaggaagctcaggtggtgccagagctggctgttagggaggagcacaggcttagttggaagaggccccatggtgtgggagctggtctcgagaacaccggtaatagctgctacctgaatgcagccctgcagtgtctgacacacacaccacctcttgccagctacatgttgtcccgggagcactctcagaactgttgtcaccgaggagcctgcatgatgtgtgctatggaagctcatgtgacccaaagtttcctctactctggggatgtcatccagccctcagagatgctgactgctgccttccacaagcacagggaggaagacgcccacgagtttctgatgttcaccttgaatgccatgcacacatcctgtctgccagggagcaagctcatgggatgcacatctaagcagagctccataatccatgagatatttggaggctcctgggaatctaagatcaagtgtctctgttgccaggcgaccacagacaccttagagcccttccttgacatcactctggatatccaaactgctcagagtgtgaaccaagccttggaaaatttagtaaaggaggaaaagctctgtggggaaaatgcctaccattgtgacatttgttggaagaacacaccggcttccaagaccctgattgtgaaagatgccccacaggttctcttgctggtgttgaatcgcttcgaagagttcacaggtgataaaaaggacagggaagtgagttactctgagttccttgacttccagccatacgtatctcagtcccctagagacccattgctttatgtcctgtatgctgtgctggtccatgatggtatgacttgtcacagcggtcattacttctgttatgtcagagctggcaatggtcactggtataagatgaatgattctagtgtcaccaggtgtgatatgaaatctgtcctaagtgagcctgcctacgtgctcttctatgtccagcagactgagctcaaaaagaatttatggatgctcccacaggcagaacaccaggcaggggaatccaggcacacaacgatcaacagaggatcccccacagaagctgaagaggccccagatcacatagagaatacaacagtccaagacttcttaggccactggaaagcgccaaagccattgacggactggaggaagaaccatcttgacagggagaatagtcccatcaggcttctgccaggcttctgtctttctcaccaggagaccatggacactgggcagctctgtagtaagggagagagaccaagatccaagaagtag

SEQ ID NO: 33 - DNA, 1449 bp, Cricetulus griseus
atgaagaaaagacgtaggcatttgcaggagggaaaggatccttctgaccacagtcagcactccagaaccatatctggaagcaaccctgaggatatggaggctgctagggagctctcagtaggtgagtctcacagtaagtcactttcagtttacatggcctcaaccaaggctgttggcactgaagtgtatttgtcttcttgccctgccacagatcctaccctgtcatctcctgacgaaccaaaccggcctcagaatgaagctcaggtggtaccagagctggctgctaaggaggagttccatcttagttggcagaggccccatgatgtgggagctggactcgagaacacaggtaatagctgctacatgaatgcagtactgcagtgtctgacacacacaccacctcttgtcaactacatgttgtctcgggagcactctcagaactgttgtcaccaaggagactgcatgatttgtgctatggaagctcatgtgacccggagtctcctctactctggggatgtcatccagccctcagagaagttgactgctgccttccacaagcacaggcaggaagatgcccatgagtttctgctgttcaccttgaatgccatgcacacatcctgtctgccagggagcaagctcctgggatgcacatctgagcagagctccctgatccatgagatatttggaggctcctggaaatctcagatcaagtgtctccactgcaatgagaccacagacctcttagagcccttccttgacatcaccctggatatccaaactgctcagagtgtgaaccaagccttggaaaatttagtaatggaggaacagctgtgtggggaaaatgcctaccattgtgacaactgtaggcagaagacaatggcttccaagaccctgactgtgaaagatgccccaaaggttctcttgctggtgttgaatcgcttctcagagttcacaggtgacaaaaaggacaggaaagtgagctatcctgagtcctttgacttccagccctacatatctcagtcccatagacaaccattgttttatagcctgtatgctgtactggtccatgatggtgtgacttgtcacagtggtcattacttctgttatgtcaaagctggcaatggtcactggtataagatggatgattctagtgtcaccagatgtgatgtcaattctgtcctaagtgaacctgcttatgtgctcttttatgtccagcagactgatctcagaacgaatttgtgggtgctctcacaggcagaacaccaggtaggggaatcctggtatacaacgatcaacagaggatcccccacagaagctgcagagcccccggatcacacagagaatacagctgccaaaaatttcttagaccactggaaaactcttctgaacatgaacaccaaagcctttggtgaaacttggaaaacacagacctactctgagagcaaatga

SEQ ID NO: 34 - DNA, 1590 bp, Nomascus leucogenys
atggagcacgactcactctactcggggggtgagtggcacttcagccgcttttcaaaactcacatcttctcggccacatgcagcttttgctgaaatccagcggacttctctccctgagaagtcaccactctcatctgagacccgtgtcgacccctgtgatgatttggctcctgtggcaagacagcttgctcccagggagaagcttcctctgagtagcaggggtcctgctgcggtgggggctgggctccagaatatgggaaatacctgctacgtgaatgcttccctgcagtgcctgacatacacaccgccccttgccaactacatgctgtcccgggagcactctcaaacttgtcatcgtcacaagtgctgcatgctctgtactatgcaagctcacatcacacgggccctccaccgtccaggcgatgtcatccagccctcacaggcattggctgctggcttccatagaggcaagcaggaagatgcccacgaatttctcatgttcactgtggatgccatgagaaaggcatgccttcccgggcacaagcaggtagatcctcactctaaggacaccaccctcatccaccaaatatttggagggtactggagatctcaaatcaagtgtctccactgccagggcatttcagacacctttgacccttacctggacatcgccctggatatccaggcagctcagagtgtgaagcaagctttggaacagttggtgaagcccgaagaactcaatggagagaatgcctatcattgtggtctttgtctccagaaggcgcctgcctccaagacgttaactttacacacttctgccaaggtcctcatcctcgtactgaagagattctccgatgtcacaggcaacaaacttgccaagaatgtgcaatatcctgagtgccttgacatgcagccatacatgtctcagcagaacacaggacctcttgtctatgtcctctacgctgtgctggtccacgctgggtggagttgtcacaacggacattacttctcttatgtcaaagctcaagaaggccagtggtataaaatggatgatgccgaggtcactgcttctggcatcacttctgtcctgagtcaacaggcctatgtcctcttttatatccagaagagtgaattggaaagacacagtgagggtgtgtcaagaggcagggaaccaagagcccttggccctgcagacacagacaggcgagcaacgcaaggagagctcaagagggaaccctgcctccaggtacccgagttggacgagcactcggtggaaagagccactcaggaaagcaccttagaccactggaaattcctccaagagcaaaacaaaacgaagcctgagttcaacgtcagaaaagtcgaaggttccctgcctcccaacgtagttgtgattcatcaatcaaaatacaagtgtggcacgaaaaaccatcatcctgaacagcaaagctccctgctaaacctctcttcgacgaacccgacagatcaggaatccatcaacactggcacacccgcttctcggcaagggaggaccaggagatccaaagggaagaacaaacacagcaacagggctctgcttctgtgccagtga

SEQ ID NO: 35 - DNA, 3360 bp, Pan troglodytes
atggaggacgactcactctacttgggaggtgagtggcagttcaaccacttttcaaaactcacatcttctcggccagatgcagcttttgctgaaatccagcggacttctctccctgagaagtcaccactctcatctgagacccgtgtcgacctctgtgatgatttggctcctgtggcaagacagcttgctcccggggagaagcttcttctgagtagcaggagacctgctgcggtgggggctgggctccagaatatgggaaatacctgctacgtgaacgcttccctgcagtgcctgacatacacaccgccccttgccaactacatgctgtcccgggagcactctcaaacgtgtcatcgtcacaagggctgcatgctctgtactatgcaagctcacatcacacgggccctccacattcctggccatgtcatccagccctcacaggcattggctgctggcttccatagaggcaagcaggaagatgcccatgaatttctcatgttcactgtggatgccatggaaaaggcatgccttcccgggcacaagcaggtagagcatcactctaaggacaccaccctcatccaccaaatatttggaggctactggagatctcaaatcaagtgtctccactgccacggcatttcagacacttttgacccttacctggacatcgccctggatatccaggcagctcagagtgtccagcaagctttggaacagttggtgaagcccgaagaactcaatggagagaatgcctatcattgtggtctttgtctccagagggcgccggcctccaagacgttaactttacacacttctgccaaggtcctcatccttgtattgaagagattctccgatgtcacaggcaacaaacttgccaagaatgtgcaatatcctgagtgccttgacatgcagccatacatgtctcagcagaacacaggacctcttgtctatgtcctctatgctgtgctggtccacgctgggtggagttgtcacaacggacattacttctcttatgtcaaagctcaagaaggccagtggtataaaatggatgatgccgaggtcaccgcctctagcatcacttctgtcctgagtcaacaggcctatgtcctcttttacatccagaagagtgaatgggaaagacacagtgagagtgcgtcaagaggcagggaaccaagagcccttggcgctgaagacacagacaggcgagcaacgcaaggagagctcaagagagaccacccctgcctgcaggcacccgagttgggcgagcacttggtggaaagagccactcaggaaagcaccttagaccactggaaattccttcaagagcaaaacaaaacgaagcctgagttcaacgtcagaaaagtcgaaggtaccctgcctcccaacgtacttgtgattcatcaatcaaaatacaagtgtgggatgaagaaccatcatcctgaacagcaaagctccctgctaaacctctcttcgacgaccccgacagatcaggagtccatgaacactggcacactcgcttccctgcaggggaggaccaggagatccaaagggaagaacaaacacagcaagagggctctgcttgtgtgccagtgatctcagtggaagtaccgacccacacatgacattcagtgtgtatttctgaatatgacctaccgacgtgtaggtttgcgtgtgaggtaaatggaggacgactcactctacttgggaggtgagtggcagttcaaccacttttcaaaactcacatcttctcggccagatgcagcttttgctgaaatccagcggacttctctccctgagaagtcaccactctcatctgagacccgtgtcgacctctgtgatgatttggctcctgtggcaagacagcttgctcccggggagaagcttcttctgagtagcaggagacctgctgcggtgggggctgggctccagaatatgggaaatacctgctacgtgaacgcttccctgcagtgcctgacatacacaccgccccttgccaactacatgctgtcccgggagcactctcaaacgtgtcatcgtcacaagggctgcatgctctgtactatgcaagctcacatcacacgggccctccacattcctggccatgtcatccagccctcacaggcattggctgctggcttccatagaggcaagcaggaagatgcccatgaatttctcatgttcactgtggatgccatggaaaaggcatgccttcccgggcacaagcaggtagagcatcactctaaggacaccaccctcatccaccaaatatttggaggctactggagatctcaaatcaagtgtctccactgccacggcatttcagacacttttgacccttacctggacatcgccctggatatccaggcagctcagagtgtccagcaagctttggaacagttggtgaagcccgaagaactcaatggagagaatgcctatcattgtggtctttgtctccagagggcgccggcctccaagacgttaactttacacacttctgccaaggtcctcatccttgtattgaagagattctccgatgtcacaggcaacaaacttgccaagaatgtgcaatatcctgagtgccttgacatgcagccatacatgtctcagcagaacacaggacctcttgtctatgtcctctatgctgtgctggtccacgctgggtggagttgtcacaacggacattacttctcttatgtcaaagctcaagaaggccagtggtataaaatggatgatgccgaggtcaccgcctctagcatcacttctgtcctgagtcaacaggcctatgtcctcttttacatccagaagagtgaatgggaaagacacagtgagagtgcgtcaagaggcagggaaccaagagcccttggcgctgaagacacagacaggcgagcaacgcaaggagagctcaagagagaccacccctgcctgcaggcacccgagttgggcgagcacttggtggaaagagccactcaggaaagcaccttagaccactggaaattccttcaagagcaaaacaaaacgaagcctgagttcaacgtcagaaaagtcgaaggtaccctgcctcccaacgtacttgtgattcatcaatcaaaatacaagtgtgggatgaagaaccatcatcctgaacagcaaagctccctgctaaacctctcttcgacgaccccgacagatcaggagtccatgaacactggcacactcgcttccctgcaggggaggaccaggagatccaaagggaagaacaaacacagcaagagggctctgcttgtgtgccagtgatctcagtggaagtaccgacccacacatgacattcagtgtgtatttctgaatatgacctaccgacgtgtaggtttgcgtgtgaggtaa

SEQ ID NO: 36 - DNA, 1910 bp, Macaca mulatta
tattatatgtgcctatcatcctgaggagtaatttgattcaggtgttctggaagtcatgatgtgggctgtgtctgttgaattcccatcgatgcaaggggacacaccctgtgactcattcctgaatggagtgctgatatttgattggtttatggcgcacctgatgagtgggtggggtgttcgcggttggtgggggtgagttctagaagggctgatgcggccagagagctcgtcatttgaagactctctcggaagagatagcgtctttctgcaacctgcggtcccagcagaaaaaccttgtgatccttgttccagtcgacatggaggacgactcactctacttgggaggtgagtggcagttcaaccacttttcaaaactcacatcttctcggccagatgcagcttttgctgaaatccagcggacttctctccctgagaagtcaccactctcatctgagacccgtgtcgacctctgtgatgatttggctcctgtggcaagacagcttgctcccagggagaagcttcctctgagtagcaggagacctgctgcggtgggggctgggctccagaatatgggaaatacctgctacgtgaacgcttccctgcagtgcctgacgtacacaccgccccttgccaactacatgctgtcccgggagcactctccaacgtgtcatcgtcacaagggctgcatgctctgtactatgcaagctcacatcacacgggccctccacattcctggccgtgtcatccagccctcacaggcattggctgctgacttccatagaggcaagcaggaagatgcccatgaatttctcatgttcactgtggatgccatgaaaaaggcatgccttcccgggcacaagcaggtagatcatcactctaaggacaccaccctcatccaccaaatatttggaggctactggagatctcaaatcaagtgtctccactgccacggcatttcagacacttttgacccttacctggacatcgccctggatatccaggcagctcagagtgtcaagcaagctttggaacagttggtgaagcccgaagaactcaatggagagaatgcctatcattgtggtctttgtctccagagggcgccggcctccaagacgttaactttacacacttctgccaaggtcctcatccttgtattgaagagattctccgatgtcacaggcagcaaacttgccaagaatgtgcactatcctgagtgccttgacatgcagccatacatgtctcagcagaacacaggacctcttgtctatgtcctctatgctgtgctggtccacgctgggtggagttgtcacaacggacattacttctcttatgtcaaagctcaagaaggccagtggtataaaatggatgatgccgaggtcaccgcctctagcatcacttctgtcctgagtcaacaggcctatgtcctcttttacatccagaagagtgaatgggaaagacacagtgagagtgcgtcaagaggcagggaaccaagagcccttggcgctgaagacacagacaggcgagcaacgcaaggagagctcaagagagaccacccctgcctgcaggcacccgagttggacgagcacttggtggaaagagccactcaggaaagcaccttagaccactggaaattccttcaagagcaaaacaaaacgaagcctgagttcaacgtcagaaaagtcgaaggtaccctgcctcccaacgtacttgtgattcatcaatcaaaatacaagtgtgggatgaagaaccatcatcctgaacagcaaagctccccgctaaacctctcttcgacgaccccgacagatcaggagtccgtgaacactggcacactcgcttccctgcaggggaggaccaggagatccaaagggaagaacaaacacagcaagagggctctgcttgtgtgccagtga

SEQ ID NO: 37 - DNA, 1490 bp, Bos taurus
atgggagccctgagcaggcaggcggcagacagagccacctgcgggggtgtggcctttgggaaggaggtcgacgtcctcagggagggggctggccctccgccgcaggtcgccccgcagacccgcagactgagggtggagcgcgaggaaccaaggttcctggggcaccgtgagcccctcctgcccttggcgcacccggccacctcccaaagacgccgcggtcccttgagggtgggggttgtgggcccagcgccgctggtgcggatgcccttcggggaccctctgtccctgagggccgtcccggcgcttcggcgtcccagcgggacaccagcagaagatgctcacgagtttctgatgttcactctgaatgccatgcagcaagggtgcttgagtgcatcccagccgtcgggtcatgcctccgaggacaccaccgtcatccgtcagatcttcggcgggacctggaggtctcagatccagtgtctccgctgcctcggtgtctcggacacgttcgacccttatctggacatcagcctggatatcacggcggctcagagtgtggagcaagctctgagagagctggtgaagcccgagaagctggacgcggacaatgcctatgactgtggcgtctgtctccggaaggtgcctgccaccaagaggttgactttgcacagcacctcccaggtcctggtgctggtgctgaagcggttcacaccggtgagcggggccaaaagggctcaggaggtgcgctatccccagtgcttggacctgcagccctacacgtccgagcggaaggcagggccactgggctacgtgctctatgccgtgctggtgcactccgggtggagctgtgagcgaggacactacttctgttacgtccgagcgggcaacggccaatggtataagatggacgatgccaaggtgaccgcctgtgacgagactgctgccctgagccagagcgcctacgtcctgttctacgcccgggagggtgcgtgggaagggggcgctgggggaggggcagcggcccccgtcggggctgaccccacagacccggggcagcctgcaggagacgccagcggcagagctcctgggtcggaggagtccccgggggacacggacgtcgaagggatgagcttagagcagtggcgacgcctgcaagaacacagccgaccgaagccggccttggagctgcggaaggtccagtctgccctgcctgccggcgcagtcgtgattcaccagtccaaacacggaggagggagaaaccgcacgccgccccaacaggagcacgagcggctcgaccgtcccagcacggacaccccgcctccggggccgaagaacgtcggcaacggcccttgtgccggcgggagggccagagccaccaaggggaagaacaagaagccgcggccgtctctggggctgtggcggtaggtcggctctgacgcacatgcgtgcagacgcccaggcacacgctgtgtggggcacgccctgtgtgacgc

SEQ ID NO: 38 - DNA, 1608 bp, canis lupus familiaris
atggaggctgcccacctccacccctcagaggagcctcagttcagcgcctctcccaaaccccagtcatactggtcaaggggaggtggtgctgaagtccacggaggaccctctgtgcccgagacgacatcccctgcatcaaagacactctcctccccgactgacccgttggctcccacatcagcagggctgcctcccaccaagacgcctctgagttggaggagcctttcccaggtgggagccgggcttcagaacatgggcaacacttgctatgtgaatgcgaccctacagtgtctgacctacacagagcccctcgccagctacatgctgtcccagcagcacgggaccacctgtaggaggcagacatcctgcatgctgtgtaccctgcaggctcacctgacgcgggttctctgccatcctggacgtgtgctccggcccctgccactcctgctcgccgccttccacagacacaagcaggaagatgcccatgagtatctcatgttcattctggatgcaatgcagcaagcgtgcttgcctgaggacaagctctcagaccctgagtgtcctcaggacagcaccctcatccagcaactctttggggggtactggaggtctcaaatccagtgtctccactgccaaggcatttcgagcactctggaaccttacctggacatcagcctggacatcggggctgctcacagcatcagccaagccttggagcagttgatgaagcccgaactgctggaaggtgaaaatgcctaccattgtagtaagtgtctggagaaggtgcctgcgtccaaggtgttgactttacacacttccccgaaggtcctcatcctggtcttgagacgattctcagacttgacaggcaacaaaatgactaaggaggtgcaatatcctgagcgccttgacatgcaacactacctgtctgagcagagggcaggacccttggtttatgtgctctatgccgtgctggtgcacgctgggaggagttgccacagcggacattacttctgtttcgtaaaggcaggaaatggccagtggtataaaatggatgatgctaaggtcagcgcctgtgatgtgacttgcgcgctacgccaacctgcctatgtcctcttttatatgcagaagactgatctggagagagaccttgggagggagtcagtcgaggagggaggactcgcatctcccgaggcagaccccacggtggtgggtgaggcctcaggagagccggcaaccgatccctccgggaaccatcctgagttggaggagcgtggggaagagacctcaaggcaacaaatgacattagaccagtggagatgcctccaagaatgcaaccgccctaagcctgaactccatgtcaggagaagagaaattgctcttcctgcgaacgcagtcatccttcaccactccaaatacagacctgagatgccgaagaatcatcctcagccgaccgtcgacctgctcaccactgcagctgggatgctcccacctcaggtggccggggacatggccaaagtcccgcgtgtgccagggagagcccgacctaccaagaggacgagcaagaagggacagaggtctggggaagcagtccagggatgtgtctcctaa

SEQ ID NO: 39 - protein, 12 aa, Artificial Sequence
  peptide derived from Dub3 protein
MSPGQLCSQGGR

SEQ ID NO: 40 - RNA, 19 nt, Artificial Sequence
  siRNA directed against cdc25A
gaaauuucccugacgagaa

SEQ ID NO: 41 - RNA, 19 nt, Artificial Sequence
  siRNA derected against Dub3
ggcuguaagaugugugcua

SEQ ID NO: 42 - RNA, 19 nt, Artificial Sequence
  derived from Dub3
uagcacacaucuuacagcc

SEQ ID NO: 43 - RNA, 10 nt, Artificial Sequence
  linker for shRNA
cuuccuguca